(19) United States
(12) Patent Application Publication
(10) Pub. No.: US 2025/0017962 A1
(43) Pub. Date: Jan. 16, 2025

(54) IGG4 HINGE-CONTAINING CHIMERIC ANTIGEN RECEPTORS TARGETING GLYPICAN-3 (GPC3) AND USE THEREOF

(71) Applicant: The U.S.A., as represented by the Secretary, Department of Health and Human Services, Bethesda, MD (US)

(72) Inventors: Mitchell Ho, Urbana, MD (US); Aarti Kolluri, Bethesda, MD (US); Nan Li, Laurel, MD (US)

(73) Assignee: The U.S.A., as represented by the Secretary, Department of Health and Human Services, Bethesda, MD (US)

(21) Appl. No.: 18/708,747

(22) PCT Filed: Nov. 9, 2022
(86) PCT No.: PCT/US2022/079554

§ 371 Date: May 9, 2024

Related U.S. Application Data
(60) Provisional application No. 63/277,287, filed on Nov. 9, 2021.

(51) Int. Cl. A61K 35/17; A61K 39/00; A61P 35/00; C07K 16/30; C12N 15/86

(52) U.S. Cl. CPC A61K 35/17; A61K 39/4611; A61K 39/4631; A61K 39/464474; A61P 35/00; C07K 16/303; C12N 15/86; A61K 2239/13; A61K 2239/21; A61K 2239/22; C12N 2740/15043

(57) ABSTRACT

Optimized chimeric antigen receptors (CARs) targeting glypican-3 (GPC3) and having a 12-amino acid hinge region derived from human IgG4 are described. The optimized CARs also include a transmembrane domain from either CD8 or CD28, an intracellular co-stimulatory domain and an intracellular signaling domain. Immune cells or induced pluripotent stem cells expressing the optimized CARs can be used to treat GPC3-positive solid tumors.

### CROSS REFERENCE TO RELATED APPLICATIONS

This application claims the benefit of U.S. Provisional Application No. 63/277,287, filed Nov. 9, 2021, which is herein incorporated by reference in its entirety.

### ACKNOWLEDGMENT OF GOVERNMENT SUPPORT

This invention was made with government support under Project No. Z01 BC010891 awarded by the National Institutes of Health. The government has certain rights in the invention.

### FIELD

This disclosure concerns optimized chimeric antigen receptors (CARs) specific for tumor antigen glypican-3 (GPC3) that include a hinge region derived from IgG4. This disclosure further concerns use of the GPC3-targeted IgG4 hinge-containing CARs, such as for treating solid tumors.

### BACKGROUND

Hepatocellular carcinoma (HCC) is a highly aggressive type of tumor with a poor prognosis. Immunotherapy using chimeric antigen receptor (CAR) engineered T lymphocytes has produced remarkable responses in hematopoietic malignancies. For solid tumors, however, significant barriers have precluded clinical success. A primary roadblock for most solid tumors is broad biomarker expression on both cancer cells and normal tissues, leading to significant toxicities. This is not the case for HCC as one of the best characterized tumor associated antigens, glypican 3 (GPC3), is highly specific for HCC. GPC3, a cell surface oncofetal protein, is highly upregulated in HCC, but is either not expressed or expressed at low levels in normal tissues. Thus, targeting GPC3 uniquely allows therapeutic trafficking exclusively to sites of HCC sites, but not normal tissue.

GPC3-targeted CAR T cells demonstrated limited efficacy in a Phase 1 clinical trial, likely due to the combination of a lack of adequate tumor penetration and an inability to induce a durable, potent immune response within the confines of the tumor. Thus, a need exists to develop CAR T cells with improved potency in vitro and in vivo.

### SUMMARY

Disclosed herein are optimized GPC3-specific chimeric antigen receptors (CARs) that include a hinge region derived from human IgG4 and a transmembrane domain from either human CD8 or human CD28. It is demonstrated herein that IgG4-hinge containing CARs targeting GPC3 specifically lyse GPC3-positive cells in vitro and are highly effective for eradicating GPC3-positive tumors in animal models.

Provided herein is a CAR that includes an extracellular antigen-binding domain specific for GPC3; an IgG4 hinge region; a transmembrane domain; an intracellular co-stimulatory domain; and an intracellular signaling domain. In some aspects, the CAR includes a hinge region consisting of the modified IgG4 hinge sequence set forth as SEQ ID NO: 43. In other aspects, the CAR includes a hinge region consisting of the wild-type IgG4 sequence set forth as SEQ ID NO: 52. In some aspects, the antigen-binding domain includes the CDR sequences of GPC3-specific single-domain antibody HN3 or the VH and VL CDR sequences of GPC3-specific antibody hYP7, YP7, YP9, YP8, YP6, YP9.1 or HS20. In some examples, the transmembrane domain of the CAR is a CD28 transmembrane domain. In other examples, the transmembrane domain of the CAR is a CD8 transmembrane domain.

Nucleic acid molecules encoding a disclosed CAR are further provided. In some aspects, the nucleic acid molecule includes in the 5′ to 3′ direction a nucleic acid encoding a first granulocyte-macrophage colony stimulating factor receptor signal sequence (GMCSFRss); a nucleic acid encoding the antigen-binding domain; a nucleic acid encoding the IgG4 hinge region; a nucleic acid encoding the transmembrane domain; a nucleic acid encoding the co-stimulatory domain; a nucleic acid encoding the signaling domain; a nucleic acid encoding a self-cleaving 2A peptide; a nucleic acid encoding a second GMCSFRss; and a nucleic acid encoding a truncated human epidermal growth factor receptor (huEGFRt). In some examples, the nucleic acid molecule further includes a human elongation factor 1α (EF1α) promoter sequence 5′ of the nucleic acid encoding the first GMCSFRss. Vectors (such as lentiviral vectors) that include the disclosed nucleic acid molecules are further provided.

Also provided are isolated immune cells (such as T cells, B cells, NK cells or macrophages) and induced pluripotent stem cells (iPSCs) expressing a CAR disclosed herein and/or containing an isolated nucleic acid molecule or vector disclosed herein.

Further provided are compositions that include a pharmaceutically acceptable carrier and a CAR, nucleic acid molecule, vector or cell disclosed herein.

Methods of treating a GPC3-positive cancer, or inhibiting tumor growth or metastasis of a GPC3-positive cancer, in a subject are also provided. In some aspects, the methods include administering to the subject a therapeutically effective amount of a CAR, nucleic acid molecule, vector, cell or composition disclosed herein. In some examples, the GPC3-positive cancer is a solid tumor, such as hepatocellular carcinoma (HCC).

The foregoing and other objects and features of the disclosure will become more apparent from the following detailed description, which proceeds with reference to the accompanying figures.

### BRIEF DESCRIPTION OF THE DRAWINGS

FIGS. 1A-1B: Jurkat binding assay. Jurkat T cells expressing hYP7-CD8H-CD8TM, HN3-CD8H-CD8TM, HN3-IgG4H-CD28TM, HN3-IgG4H-CD8TM or HN3-CD8H-CD28TM CAR were exposed to GPC3-hFc (FIG. 1A) or GPC1-hFc (FIG. 1B). All hYP7 and HN3 CAR-expressing Jurkat cells specifically bound GPC3-hFc.

FIGS. 2A-2D: (FIG. 2A) Schematic of CAR T cell constructs. The constructs included either HN3or hYP7 with a CD8 or IgG4 hinge (H), and a CD8 or CD28 transmembrane (TM) domain. (FIG. 2B) Depiction of CAR T cell constructs in a cell membrane. (FIG. 2C) Cell count of CAR-expressing cells monitored over the course of 11 days. (FIG. 2D) Transduction efficiency of CAR constructs as measured by CAR-positive cells at Day 8. These results demonstrate that the engineered CARs can be expressed in donor T cells.

FIGS. 2E-2H: Graphs showing cell killing induced by CAR T cells cultured with GPC3-expressing HCC cell lines Hep3B (FIG. 2E), Huh7 (FIG. 2G) and HepG2 (FIG. 2H) or GPC3-negative Hep3B-GPC3-KO-C3 cells (FIG. 2F). Specific lysis was measured at different effector to target ratios. Engineered CAR T cells potently killed Hep3B, Huh7 and HepG2 cells, but not Hep3B GPC3 knockout (KO) cells, demonstrating antigen-specific killing.

FIGS. 3A-3D: (FIG. 3A) Schematic of the experimental design of a study using the NOD-SCID-Gamma (NSG) mouse model. Hep3B GFP/luciferase expressing cells (3 million) were injected IP into NSG mice and allowed to engraft for 12 days. Mice were treated with 5 million hYP7-CD8H-CD8TM, hYP7-IgG4H-CD28TM, HN3-IgG4H-CD8TM or HN3-IgG4H-CE28TM CAR T cells on Day 0 and imaged regularly. (FIG. 3B) Bioluminescence images showing tumor size. (FIG. 3C) Bioluminescence quantification of imaging results over 35 days. (FIG. 3D) Survival curve over the full course of the study. The results showed that within 10 days, HN3-IgG4H-CD28TM CAR T cells completely eradicated tumors, and mice remained tumor free over the full course of the study.

FIGS. 4A-4D: (FIG. 4A) Schematic of the experimental design of a study using the NSG mouse model. Hep3B GFP/luciferase expressing cells (3 million) were injected IP into NSG mice and allowed to engraft for 12 days. Mice were treated with 5 million HN3-CD8H-CD8TM, HN3-IgG4H-CD28TM, HN3-lgG4H-CD28TM-M or HN3-IgG4H-CD28TM-L CAR T cells on Day 0 and imaged regularly. (FIG. 4B) Bioluminescence imaging results showing tumor size. (FIG. 4C) Bioluminescence quantification of imaging results over 15 days. (FIG. 4D) Survival curve over the full course of the study (67 days). The results showed that within 7 days, the HN3-IgG4-CD28TM CAR T cells eliminated tumors and treated mice remained tumor free over the study period. Additionally, mice treated with HN3-IgG4H-CD28TM CAR T cells exhibited the greatest survival with approximately 40% of mice in this group surviving for at least the 67 day study period and remaining tumor free.

FIGS. 5A-5F: (FIGS. 5A-5B) Analysis of CD4 and CD8 T cells and T cell subsets. T cell subsets consist of stem cell memory (T_{scm}), central memory (T_{cm}), effector memory re-expressing CD45RA (Tₑₘᵣₐ), and effector memory cells (Tₑₘ). (FIG. 5A) Week 2 T cells appeared similar in CD4/CD8 ratio. Tₑₘᵣₐ and Tₑₘ dominated in the T cell landscape. (FIG. 5B) Week 5 CD8 T cells were most the abundant. Tₑₘᵣₐ cells were enriched and engaged in proliferative, memory, and effector functions. (FIG. 5C) Time course of CAR T cell counts from mouse blood. (FIG. 5D) Time course of PD1 expression of T cells from mouse blood. (FIG. 5E) Exhaustion marker panel at week 5. (FIG. 5F) Hep3B tumor cell killing induced by CAR-expressing T cells. The HN3-IgG4H-CD28TM construct induced the greatest tumor cell lysis.

FIGS. 6A-6D: (FIG. 6A) Schematic of the experimental design of a study using the NSG mouse model. Huh7 GFP/luciferase expressing cells were injected IP into mice (Day—12) and allowed to engraft for 12 days. Mice were injected IP with 15 million untransduced T cells or hYP7-CD8H-CD8TM or HN3-IgG4H-CD28TM CAR T cells on Day 0 and imaged regularly for four weeks. (FIG. 6B) Bioluminescence imaging results showing tumor size. (FIG. 6C) Survival curve over the full course of the study (28 days post-treatment). (FIG. 6D) Tumor volume at the end of the 28-day study.

FIGS. 7A-7B: (FIG. 7A) Western blot of active and total β catenin, GPC3 and GAPDH in tumor cells exposed to CAR T cells at 30 minutes and 3 hours. (FIG. 7B) NFAT signaling in CAR T cells when CAR T cells are cultured with Hep3B tumor cells.

### SEQUENCE LISTING

The nucleic and amino acid sequences listed in the accompanying sequence listing are shown using standard letter abbreviations for nucleotide bases, and single letter code for amino acids, as defined in 37C.F.R. 1.822. Only one strand of each nucleic acid sequence is shown, but the complementary strand is understood as included by any reference to the displayed strand. The Sequence Listing is submitted as an XML file, named 4239-107164-02.xml (91,430 bytes), created on Oct. 25, 2022, which is incorporated by reference herein. In the accompanying sequence listing:
SEQ ID NO: 1 is a nucleic acid sequence encoding HN3-CD8H-CD8TM.
SEQ ID NO: 2 is the amino acid sequence of HN3-CD8H-CD8TM.
SEQ ID NO: 3 is a nucleic acid sequence encoding HN3-CD8H-CD28TM.
SEQ ID NO: 4 is the amino acid sequence of HN3-CD8H-CD28TM.
SEQ ID NO: 5 is a nucleic acid sequence encoding HN3-IgG4H-CD28TM.
SEQ ID NO: 6 is the amino acid sequence of HN3-IgG4H-CD28TM.
SEQ ID NO: 7 is a nucleic acid sequence encoding HN3-IgG4H-CD8TM.
SEQ ID NO: 8 is the amino acid sequence of HN3-IgG4H-CD8TM.
SEQ ID NO: 9 is a nucleic acid sequence encoding HN3-IgG4H-CH3-CD28TM.
SEQ ID NO: 10 is the amino acid sequence of HN3-IgG4H-CH3-CD28TM.
SEQ ID NO: 11 is a nucleic acid sequence encoding HN3-IgG4H-CH2CH3-CD28TM.
SEQ ID NO: 12 is the amino acid sequence of HN3-IgG4H-CH2CH3-CD28TM.
SEQ ID NO: 13 is a nucleic acid sequence encoding hYP7-IgG4H-CD28TM.
SEQ ID NO: 14 is the amino acid sequence of hYP7-IgG4H-CD28TM.
SEQ ID NO: 15 is a nucleic acid sequence encoding hYP7-CD8H-CD8TM.
SEQ ID NO: 16 is the amino acid sequence of hYP7-CD8H-CD8TM.
SEQ ID NO: 17 is a nucleic acid sequence encoding antibody HN3.
SEQ ID NO: 18 is the amino acid sequence of antibody HN3.
SEQ ID NO: 19 is a nucleic acid sequence encoding the VH domain of antibody hYP7.
SEQ ID NO: 20 is the amino acid sequence of the VH domain of antibody hYP7.
SEQ ID NO: 21 is a nucleic acid sequence encoding the VL domain of antibody hYP7.
SEQ ID NO: 22 is the amino acid sequence of the VL domain of antibody hYP7.
SEQ ID NO: 23 is a nucleic acid sequence encoding the VH domain of antibody YP9.1.
SEQ ID NO: 24 is the amino acid sequence of the VH domain of antibody YP9.1.
SEQ ID NO: 25 is a nucleic acid sequence encoding the VL domain of antibody YP9.1.
SEQ ID NO: 26 is the amino acid sequence of the VL domain of antibody YP9.1.
SEQ ID NO: 27 is a nucleic acid sequence encoding the VH domain of antibody YP8.
SEQ ID NO: 28 is the amino acid sequence of the VH domain of antibody YP8.
SEQ ID NO: 29 is a nucleic acid sequence encoding the VL domain of antibody YP8.
SEQ ID NO: 30 is the amino acid sequence of the VL domain of antibody YP8.
SEQ ID NO: 31 is a nucleic acid sequence encoding the VH domain of antibody YP9.
SEQ ID NO: 32 is the amino acid sequence of the VH domain of antibody YP9.
SEQ ID NO: 33 is a nucleic acid sequence encoding the VL domain of YP9 clone 9.
SEQ ID NO: 34 is the amino acid sequence of the VL domain of YP9 clone 9.
SEQ ID NO: 35 is a nucleic acid sequence encoding the VL domain of YP9 clone 10.
SEQ ID NO: 36 is the amino acid sequence of the VL domain of YP9 clone 10.
SEQ ID NO: 37 is a nucleic acid sequence encoding the VL domain of YP9 clone 1.
SEQ ID NO: 38 is the amino acid sequence of the VL domain of YP9 clone 1.
SEQ ID NO: 39 is a nucleic acid sequence encoding the VH domain of antibody HS20.
SEQ ID NO: 40 is the amino acid sequence of the VH domain of antibody HS20.
SEQ ID NO: 41 is a nucleic acid sequence encoding the VL domain of antibody HS20.
SEQ ID NO: 42 is the amino acid sequence of the VL domain of antibody HS20.
SEQ ID NO: 43 is the amino acid sequence of a modified IgG4 hinge region.
SEQ ID NO: 44 is the amino acid sequence of GMSCFRss.
SEQ ID NO: 45 is the amino acid sequence of the CD8α hinge.
SEQ ID NO: 46 is the amino acid sequence of the CD28 transmembrane domain.
SEQ ID NO: 47 is the amino acid sequence of the CD8α transmembrane domain.
SEQ ID NO: 48 is the amino acid sequence of 4-1BB
SEQ ID NO: 49 is the amino acid sequence of CD3ζ.
SEQ ID NO: 50 is the amino acid sequence of the self-cleaving T2A peptide
SEQ ID NO: 51 is the amino acid sequence of huEGFRt.
SEQ ID NO: 52 is the amino acid sequence of a wild-type IgG4 hinge region.
SEQ ID NO: 53 is a nucleic acid sequence encoding the VH domain of antibody YP7.
SEQ ID NO: 54 is the amino acid sequence of the VH domain of antibody YP7.
SEQ ID NO: 55 is a nucleic acid sequence encoding the VL domain of antibody YP7.
SEQ ID NO: 56 is the amino acid sequence of the VL domain of antibody YP7.
SEQ ID NO: 57 is a nucleic acid sequence encoding the VH domain of antibody YP6.
SEQ ID NO: 58 is the amino acid sequence of the VH domain of antibody YP6.
SEQ ID NO: 59 is the amino acid sequence of a modified CH2 domain.
SEQ ID NO: 60 is the amino acid sequence of a wild-type CH2 domain.

### DETAILED DESCRIPTION

### I. Abbreviations

CAR chimeric antigen receptor
CDR complementarity determining region
EF1α elongation factor 1 alpha
EGF epidermal growth factor
EGFR epidermal growth factor receptor
GPC3 glypican-3
GMCSFRss granulocyte-macrophage colony stimulating factor receptor signal sequence
HCC hepatocellular carcinoma
huEGFRt human truncated epidermal growth factor receptor
IP intraperitoneal
iPSC induced pluripotent stem cell
KO knockout
NK natural killer
NSG NOD-SCID-Gamma
scFv single-chain variable fragment
TM transmembrane
T_{cm} central memory T cell
Tₑₘ effector memory T cell
Tₑₘᵣₐ effector memory T cell re-expressing CD45RA
T_{scm} stem cell memory T cell
VH variable heavy
VL variable light

### II. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “an antigen” includes singular or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting.

To facilitate review of the various aspects, the following explanations of terms are provided:

4-1BB: A co-stimulatory molecule expressed by T cell receptor (TCR)-activated lymphocytes, and by other cells including natural killer cells. Ligation of 4-1BB induces a signaling cascade that results in cytokine production, expression of anti-apoptotic molecules and an enhanced immune response. An exemplary amino acid sequence of 4-1BB is set forth herein as SEQ ID NO: 48.

Administration: To provide or give a subject an agent, such as a CAR or CAR-expressing cell provided herein, by any effective route. Exemplary routes of administration include, but are not limited to, oral, injection (such as subcutaneous, intramuscular, intradermal, intraperitoneal, intravenous, intra-arterial (including hepatic intra-arterial), intraprostatic, and intratumoral), sublingual, rectal, transdermal, intranasal, vaginal and inhalation routes. In some examples administration is local. In some examples administration is systemic.

Antibody: A polypeptide ligand comprising at least one variable region that recognizes and binds (such as specifically recognizes and specifically binds) an epitope of an antigen, such as GPC3. Mammalian immunoglobulin molecules are composed of a heavy (H) chain and a light (L) chain, each of which has a variable region, termed the variable heavy (V_{H}) region and the variable light (V_{L}) region, respectively. Together, the V_{H} region and the V_{L} region are responsible for binding the antigen recognized by the antibody. There are five main heavy chain classes (or isotypes) of mammalian immunoglobulin, which determine the functional activity of an antibody molecule: IgM, IgD, IgG, IgA and IgE. Antibody isotypes not found in mammals include IgX, IgY, IgW and IgNAR. IgY is the primary antibody produced by birds and reptiles, and has some functionally similar to mammalian IgG and IgE. IgW and IgNAR antibodies are produced by cartilaginous fish, while IgX antibodies are found in amphibians.

Antibody variable regions contain “framework” regions and hypervariable regions, known as “complementarity determining regions” or “CDRs.” The CDRs are primarily responsible for binding to an epitope of an antigen. The framework regions of an antibody serve to position and align the CDRs in three-dimensional space. The amino acid sequence boundaries of a given CDR can be readily determined using any of a number of numbering schemes, including those described by Kabat et al. (Sequences of Proteins of Immunological Interest, U.S. Department of Health and Human Services, 1991; the “Kabat” numbering scheme), Chothia et al. (see Chothia and Lesk, J Mol Biol 196:901-917, 1987; Chothia et al., Nature 342:877, 1989; and Al-Lazikani et al., (JMB 273,927-948, 1997; the “Chothia” numbering scheme), and the ImMunoGeneTics (IMGT) database (see, Lefranc, Nucleic Acids Res 29:207-9, 2001; the “IMGT” numbering scheme). The Kabat and IMGT databases are maintained online.

A “single-domain antibody” refers to an antibody having a single domain (a variable domain) that is capable of specifically binding an antigen, or an epitope of an antigen, in the absence of an additional antibody domain. Single-domain antibodies include, for example, V_{H} domain antibodies, V_{NAR} antibodies, camelid V_{H}H antibodies, and V_{L} domain antibodies. V_{NAR} antibodies are produced by cartilaginous fish, such as nurse sharks, wobbegong sharks, spiny dogfish and bamboo sharks. Camelid V_{H}H antibodies are produced by several species including camel, llama, alpaca, dromedary, and guanaco, which produce heavy chain antibodies that are naturally devoid of light chains.

A “monoclonal antibody” is an antibody produced by a single clone of lymphocytes or by a cell into which the coding sequence of a single antibody has been transfected. Monoclonal antibodies are produced by known methods. Monoclonal antibodies include humanized monoclonal antibodies.

A “chimeric antibody” has framework residues from one species, such as human, and CDRs (which generally confer antigen binding) from another species.

A “humanized” antibody is an immunoglobulin including a human framework region and one or more CDRs from a non-human (for example a mouse, rabbit, rat, shark or synthetic) immunoglobulin. The non-human immunoglobulin providing the CDRs is termed a “donor,” and the human immunoglobulin providing the framework is termed an “acceptor.” In one aspect, all CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A humanized antibody binds to the same antigen as the donor antibody that provides the CDRs. Humanized or other monoclonal antibodies can have additional conservative amino acid substitutions which have substantially no effect on antigen binding or other immunoglobulin functions.

Binding affinity: Affinity of an antibody or other antigen-binding molecule for an antigen. In one aspect, affinity is calculated by a modification of the Scatchard method described by Frankel et al., Mol. Immunol., 16:101-106, 1979. In another aspect, binding affinity is measured by an antigen/antibody dissociation rate. In another aspect, a high binding affinity is measured by a competition radioimmunoassay. In another aspect, binding affinity is measured by ELISA. In some aspects, binding affinity is measured using the Octet system (Creative Biolabs), which is based on bio-layer interferometry (BLI) technology. In other aspects, Kd is measured using surface plasmon resonance assays using a BIACORES-2000 or a BIACORES-3000 (BIAcore, Inc., Piscataway, N.J.). In other aspects, antibody affinity is measured by flow cytometry. An antibody or CAR that “specifically binds” an antigen (such as GPC3) is an antibody or CAR that binds the antigen with high affinity and does not significantly bind other unrelated antigens.

Chemotherapeutic agent: Any chemical agent with therapeutic usefulness in the treatment of diseases characterized by abnormal cell growth. Such diseases include tumors, neoplasms, and cancer. In one aspect, a chemotherapeutic agent is an agent of use in treating a GPC3-positive tumor. In one aspect, a chemotherapeutic agent is a radioactive compound. Exemplary chemotherapeutic agents that can be used with the methods provided herein are disclosed in Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., © 2000 Churchill Livingstone, Inc; Baltzer, L., Berkery, R. (eds.): Oncology Pocket Guide to Chemotherapy, 2nd ed. St. Louis, Mosby-Year Book, 1995; Fischer, D. S., Knobf, M. F., Durivage, H. J. (eds): The Cancer Chemotherapy Handbook, 4th ed. St. Louis, Mosby-Year Book, 1993). Combination chemotherapy is the administration of more than one agent to treat cancer. One example is the administration of GPC3-targeted CAR T cells used in combination with a radioactive or chemical compound. In one example, a chemotherapeutic agent is a biologic, such as a therapeutic antibody (e.g., therapeutic monoclonal antibody), such as an anti-GPC3 antibody, as well as other anti-cancer antibodies, such as anti-PD1 or anti-PDL1 (e.g., pembrolizumab and nivolumab), anti-CTLA4 (e.g., ipilimumab), anti-EGFR (e.g., cetuximab), anti-VEGF (e.g., bevacizumab), or combinations thereof (e.g., anti-PD-1 and anti-CTLA-4). Combination chemotherapy is the administration of more than one agent to treat cancer.

Chimeric antigen receptor (CAR): A chimeric molecule that includes an antigen-binding portion (such as single-domain antibody or scFv) and a signaling domain, such as a signaling domain from a T cell receptor (for example, CD3ζ). In many instances, CARs are comprised of an antigen-binding moiety, a hinge region, a transmembrane domain and an endodomain. The endodomain typically includes a signaling chain having an immunoreceptor tyrosine-based activation motif (ITAM), such as CD3ζ or FeERIγ. In some cases, the endodomain further includes the intracellular portion of at least one additional co-stimulatory domain, such as CD28, 4-1BB (CD137), ICOS, OX40 (CD134), CD27, MYD88-CD40, KIR2DS2 and/or DAP10. In some examples, the CAR is multispecific (such as bispecific) or bicistronic. A multispecific CAR is a single CAR molecule comprised of at least two antigen-binding domains (such as scFvs and/or single-domain antibodies) that each bind a different antigen or a different epitope on the same antigen (see, for example, US 2018/0230225). For example, a bispecific CAR refers to a single CAR molecule having two antigen-binding domains that each bind a different antigen. A bicistronic CAR refers to two complete CAR molecules, each containing an antigen-binding moiety that binds a different antigen. In some cases, a bicistronic CAR construct expresses two complete CAR molecules that are linked by a cleavage linker. Immune cells (such as T cells, B cells, NK cells or macrophages) or iPSCs expressing a bispecific or bicistronic CAR can bind cells that express both of the antigens to which the binding moieties are directed (see, for example, Qin et al., Blood 130:810, 2017; and WO 2018/213337). In some aspects, the CAR is a two-chained antibody-T cell receptor (AbTCR) as described in Xu et al. (Cell Discovery 4:62, 2018) or a synthetic T cell receptor and antigen receptor (STAR) as described by Liu et al. (Sci Transl Med 13(586):eabb5191, 2021).

Complementarity determining region (CDR): A region of hypervariable amino acid sequence that defines the binding affinity and specificity of an antibody. The light and heavy chains of a mammalian immunoglobulin each have three CDRs, designated L-CDR1, L-CDR2, L-CDR3 and H-CDR1, H-CDR2, H-CDR3, respectively. A single-domain antibody contains three CDRs, referred to herein as CDR1, CDR2 and CDR3.

Conservative variant: In the context of the present disclosure, “conservative” amino acid substitutions are those substitutions that do not substantially affect or decrease the affinity of a protein, such as an antibody, to GPC3. As one example, a monoclonal antibody that specifically binds GPC3 can include at most about 1, at most about 2, at most about 5, and most about 10, or at most about 15 conservative substitutions and specifically bind the GPC3 polypeptide. The term “conservative variant” also includes the use of a substituted amino acid in place of an unsubstituted parent amino acid, provided that the variant retains activity. Non-conservative substitutions are those that reduce an activity (such as affinity) of a protein.

Conservative amino acid substitution tables providing functionally similar amino acids are known. The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

In some aspects herein, provided are amino acid sequences comprising no more than 10, no more than 9, no more than 8, no more than 7, no more than 6, no more than 5, no more than 4, no more than 3, no more than 2 or no more than 1 amino acid substitutions relative to any amino acid sequence disclosed herein.

Contacting: Placement in direct physical association; includes both in solid and liquid form.

Degenerate variant: A polynucleotide encoding a polypeptide that includes a sequence that is degenerate as a result of the genetic code. There are 20 natural amino acids, most of which are specified by more than one codon. Therefore, all degenerate nucleotide sequences are included as long as the amino acid sequence of the polypeptide is unchanged.

Epitope: An antigenic determinant. These are particular chemical groups or peptide sequences on a molecule that are antigenic (that elicit a specific immune response). An antibody specifically binds a particular antigenic epitope on a polypeptide.

Framework region: Amino acid sequences interposed between CDRs. Framework regions include variable light and variable heavy framework regions. The framework regions serve to hold the CDRs in an appropriate orientation for antigen binding.

Fusion protein: A protein comprising at least a portion of two different (heterologous) proteins.

Glypican-3 (GPC3): A member of the glypican family of heparan sulfate (HS) proteoglycans that are attached to the cell surface by a glycosylphosphatidylinositol anchor (Filmus and Selleck, J Clin Invest 108:497-501, 2001). The GPC3 gene codes for a core protein of approximately 70 kD, which can be cleaved by furin to produce an N-terminal 40 kD fragment and a C-terminal 30 kD fragment. Two HS chains are attached on the C-terminal portion of GPC3. GPC3 and other glypican family proteins play a role in cell division and cell growth regulation. GPC3 is highly expressed in HCC and some other human cancers including melanoma, squamous cell carcinomas of the lung, and clear cell carcinomas of the ovary (Ho and Kim, Eur J Cancer 47(3):333-338, 2011), but is not expressed in normal tissues. GPC3 is also known as SGB, DGSX, MXR7, SDYS, SGBS, OCI-5, SGBS1 and GTR2-2.

There are four known isoforms of human GPC3 (isoforms 1-4) (Ho and Kim, Eur J Cancer 47(3):333-338, 2011). Nucleic acid and amino acid sequences of the four isoforms of GPC3 are known, including GenBank Accession numbers: NM_001164617 and NP_001158089 (isoform 1); NM_004484 and NP_004475 (isoform 2); NM_001164618 and NP_001158090 (isoform 3); and NM_001164619 and NP_001158091 (isoform 4).

GPC3-positive cancer: A cancer that expresses or overexpresses GPC3. Examples of GPC3-positive cancers include, but are not limited to, HCC, melanoma, ovarian clear-cell carcinomas, yolk sac tumors (YST), neuroblastoma, hepatoblastoma, Wilms' tumors, squamous cell carcinoma of the lung, testicular nonseminomatous germ cell tumors, liposarcoma, cervical intraepithelial neoplasia, adenoma of the adrenal gland, schwannoma and embryonal tumors (Ho and Kim, Eur J Cancer 47(3):333-338, 2011; Baumhoer et al., Am J Clin Pathol 129(6):899-906, 2008; Saikali and Sinnett, Int J Cancer 89(5):418-422, 2000).

Hepatocellular carcinoma (HCC): A primary malignancy of the liver typically occurring in patients with inflammatory livers resulting from viral hepatitis, liver toxins or hepatic cirrhosis (often caused by alcoholism). HCC is also called malignant hepatoma.

Heterologous: Originating from a separate genetic source or species.

Host cell: Cells in which a vector can be propagated and its DNA expressed. The cell may be prokaryotic or eukaryotic. In some examples, the prokaryotic cell is an E. coli cell. In some examples, the eukaryotic cell is a human cell, such as a human embryonic kidney (HEK) cell. The term also includes any progeny of the subject host cell. It is understood that all progeny may not be identical to the parental cell since there may be mutations that occur during replication. However, such progeny are included when the term “host cell” is used.

Immune response: A response of a cell of the immune system, such as a B cell, T cell, or monocyte, to a stimulus. In one aspect, the response is specific for a particular antigen (an “antigen-specific response”). In one aspect, an immune response is a T cell response, such as a CD4⁺ response or a CD8⁺ response. In another aspect, the response is a B cell response, and results in the production of specific antibodies.

Isolated: An “isolated” biological component, such as a nucleic acid, protein (including antibodies) or organelle, has been substantially separated or purified away from other biological components in the environment (such as a cell) in which the component occurs, e.g., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Label: A detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a “labeled antibody” refers to incorporation of another molecule in the antibody. For example, the label is a detectable marker, such as the incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Various methods of labeling polypeptides and glycoproteins are known and may be used. Examples of labels for polypeptides include, but are not limited to, the following: radioisotopes or radionucleotides (such as ³⁵S, ¹¹C, ¹³N, ¹⁵O, ¹⁸F, ¹⁹F, ^{99m}Tc, ¹³¹I, ³H, ¹⁴C, ¹⁵N, ⁹⁰Y, ⁹⁹Tc, ¹¹¹In and ¹²⁵I), fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as leucine zipper pair sequences, binding sites for secondary antibodies, metal binding domains, epitope tags), or magnetic agents, such as gadolinium chelates. In some aspects, labels are attached by spacer arms of various lengths to reduce potential steric hindrance.

Linker: In some cases, a linker is a peptide within an antibody binding fragment (such as an Fv fragment) which serves to indirectly bond the variable heavy chain to the variable light chain. “Linker” can also refer to a peptide serving to link a targeting moiety, such as an antibody, to an effector molecule, such as a cytotoxin or a detectable label. The terms “conjugating,” “joining,” “bonding” or “linking” refer to making two polypeptides into one contiguous polypeptide molecule, or to covalently attaching a radionuclide or other molecule to a polypeptide, such as an scFv. In the specific context, the terms include reference to joining a ligand, such as an antibody moiety, to an effector molecule. The linkage can be either by chemical or recombinant means. “Chemical means” refers to a reaction between the antibody moiety and the effector molecule such that there is a covalent bond formed between the two molecules to form one molecule.

Mammal: This term includes both human and non-human mammals. Similarly, the term “subject” includes both human and veterinary subjects, such as mice, rats, cows, cats, dogs, pigs, and non-human primates.

Melanoma: A form of cancer that originates in melanocytes (cells that make the pigment melanin). Melanocytes are found primarily in the skin, but are also present in the bowel and eye. Melanoma in the skin includes superficial spreading melanoma, nodular melanoma, acral lentiginous melanoma, and lentigo maligna (melanoma). Any of the above types may produce melanin or can be amelanotic. Similarly, any subtype may show desmoplasia (dense fibrous reaction with neurotropism) which is a marker of aggressive behavior and a tendency to local recurrence. Other melanomas include clear cell sarcoma, mucosal melanoma and uveal melanoma.

Neoplasia, malignancy, cancer or tumor: A neoplasm is an abnormal growth of tissue or cells that results from excessive cell division. Neoplastic growth can produce a tumor. The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.”

Neuroblastoma: A solid tumor arising from embryonic neural crest cells. Neuroblastoma commonly arises in and around the adrenal glands, but can occur anywhere that sympathetic neural tissue is found, such as in the abdomen, chest, neck or nerve tissue near the spine. Neuroblastoma typically occurs in children younger than 5 years of age.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Ovarian cancer: Cancer that forms in tissues of the ovary (one of a pair of female reproductive glands in which the ova, or eggs, are formed). Most ovarian cancers are either ovarian epithelial carcinomas (cancer that begins in the cells on the surface of the ovary) or malignant germ cell tumors (cancer that begins in egg cells).

Ovarian clear cell carcinoma: A distinct histopathologic subtype of epithelial ovarian cancer with an incidence of less than 5% of all ovarian malignancies. When viewed under a microscope, the insides of the cells of this type of tumor appear clear.

Pharmaceutically acceptable carriers: The pharmaceutically acceptable carriers of use are conventional. Remington: The Science and Practice of Pharmacy, 22^{nd} ed., London, UK: Pharmaceutical Press, 2013,), describes compositions and formulations suitable for pharmaceutical delivery of the CAR-expressing immune cells and other compositions disclosed herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop, such as a reduction in tumor burden or a decrease in the number or size of metastases. “Ameliorating” refers to the reduction in the number or severity of signs or symptoms of a disease, such as cancer.

Purified: The term purified does not require absolute purity; rather, it is intended as a relative term. Thus, for example, a purified peptide preparation is one in which the peptide or protein is more enriched than the peptide or protein is in its natural environment within a cell. In one aspect, a preparation is purified such that the protein or peptide represents at least 50% of the total peptide or protein content of the preparation. Substantial purification denotes purification from other proteins or cellular components. A substantially purified protein is at least 60%, 70%, 80%, 90%, 95% or 98% pure. Thus, in one specific, non-limiting example, a substantially purified protein is 90% free of other proteins or cellular components.

Recombinant: A recombinant nucleic acid is one that has a sequence that is not naturally occurring or has a sequence that is made by an artificial combination of two otherwise separated segments of sequence. This artificial combination is often accomplished by chemical synthesis or by the artificial manipulation of isolated segments of nucleic acids, for example, by genetic engineering techniques.

Sample (or biological sample): A biological specimen containing genomic DNA, RNA (including mRNA), protein, or combinations thereof, obtained from a subject. Examples include, but are not limited to, peripheral blood, tissue, cells, urine, saliva, tissue biopsy, fine needle aspirate, surgical specimen, and autopsy material. In one example, a sample includes a tumor biopsy, such as a tumor tissue biopsy.

Sequence identity: The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide or nucleic acid molecule will possess a relatively high degree of sequence identity when aligned using standard methods.

Methods of alignment of sequences for comparison are known. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Homologs and variants of an antibody or CAR that specifically binds GPC3 are typically characterized by possession of at least about 75%, for example at least about 80%, 90%, 95%, 96%, 97%, 98% or 99% sequence identity counted over the full-length alignment with the amino acid sequence of the antibody or CAR using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. These sequence identity ranges are provided for guidance only; it is entirely possible that strongly significant homologs could be obtained that fall outside of the ranges provided.

Squamous cell carcinoma: A type of cancer that originates in squamous cells, thin, flat cells that form the surface of the skin, eyes, various internal organs, and the lining of hollow organs and ducts of some glands. Squamous cell carcinoma is also referred to as epidermoid carcinoma. One type of squamous cell carcinoma is squamous cell carcinoma of the lung. Squamous cell carcinoma is the most common type of skin cancer.

Subject: Living multi-cellular vertebrate organisms, a category that includes both human and veterinary subjects, including human and non-human mammals such as pigs, mice, rats, rabbits, sheep, horses, cows, dogs, cats and non-human primates.

Synthetic: Produced by artificial means in a laboratory, for example a synthetic nucleic acid or protein (for example, an antibody) can be chemically synthesized in a laboratory.

Therapeutically effective amount: A quantity of a specific substance sufficient to achieve a desired effect in a subject being treated. For instance, this can be the amount necessary to inhibit or suppress growth of a tumor. In one aspect, a therapeutically effective amount is the amount necessary to eliminate, reduce the size, or prevent metastasis of a tumor, such as reduce a tumor size and/or volume by at least 10%, at least 20%, at least 50%, at least 75%, at least 80%, at least 90%, at least 95%, or even 100%, and/or reduce the number and/or size/volume of metastases by at least 10%, at least 20%, at least 50%, at least 75%, at least 80%, at least 90%, at least 95%, or even 100%, for example as compared to a size/volume/number prior to treatment,. When administered to a subject, a dosage will generally be used that will achieve target tissue concentrations (for example, in tumors) that has been shown to achieve a desired in vitro effect.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other known genetic elements. In some examples, the vector is a viral vector, such as a lentiviral vector, an adenovirus vector, or an adeno-associated virus (AAV) vector.

### III. Overview of Several Aspects

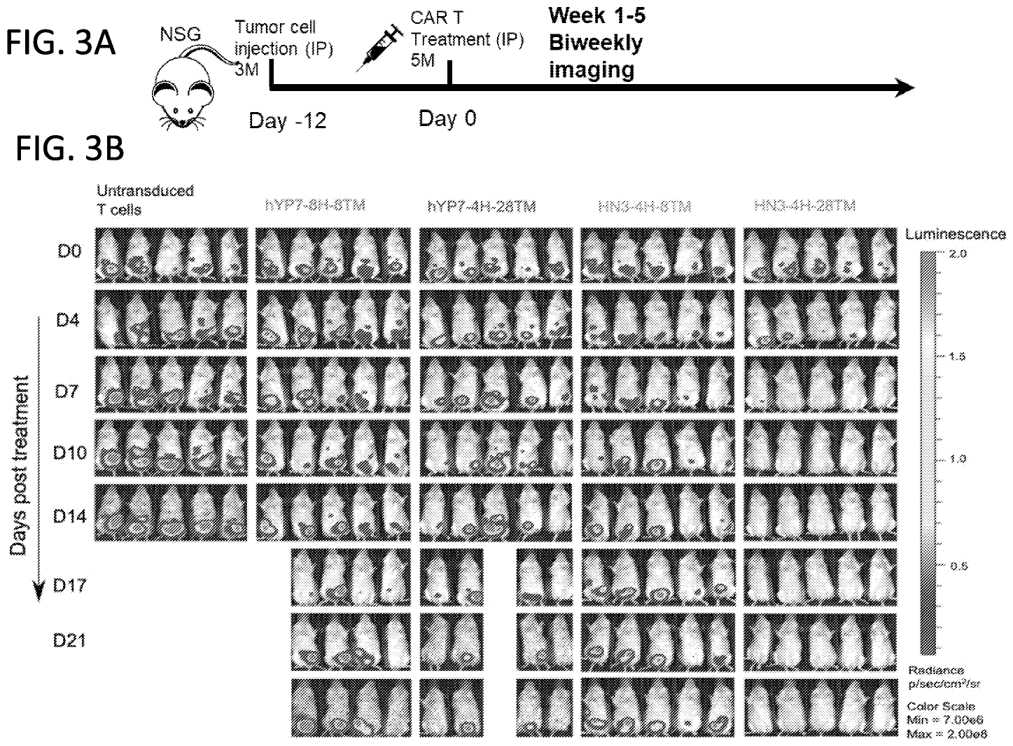

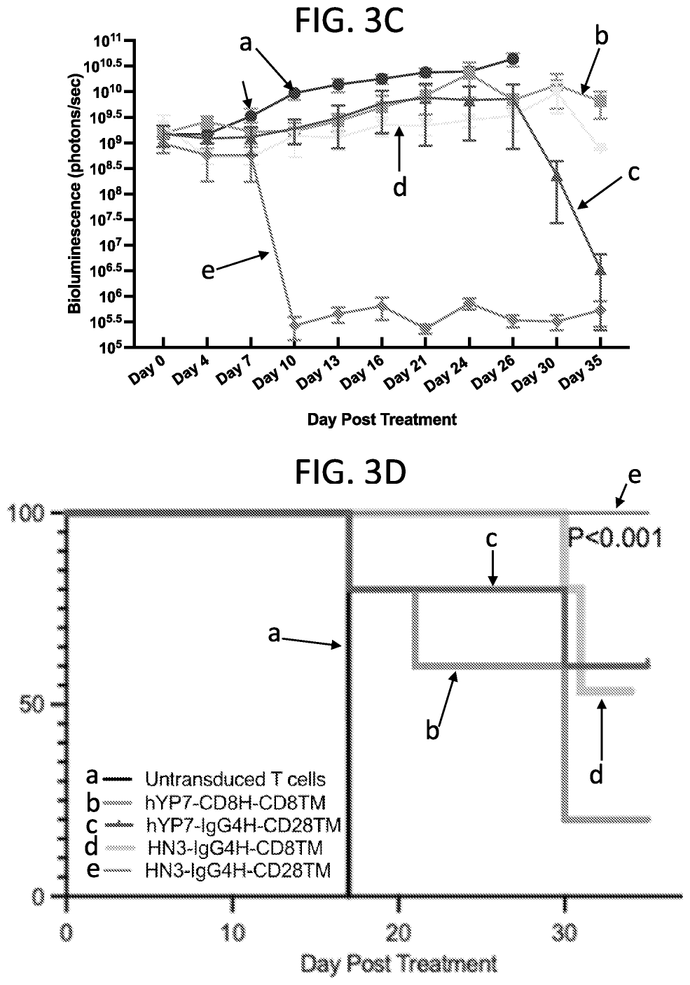

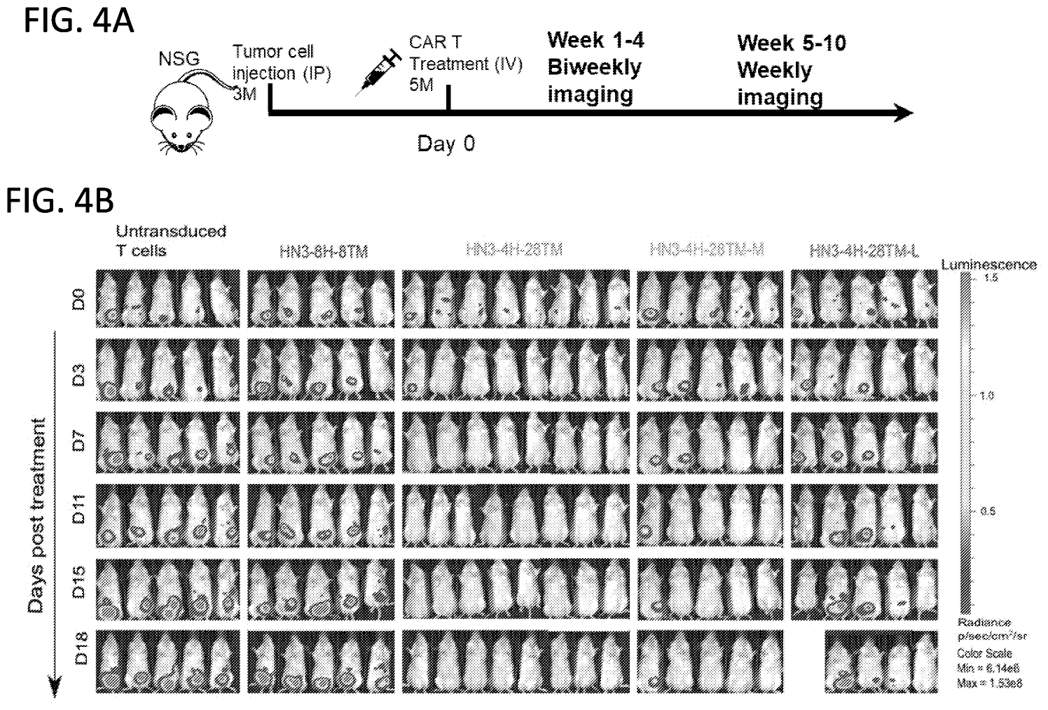

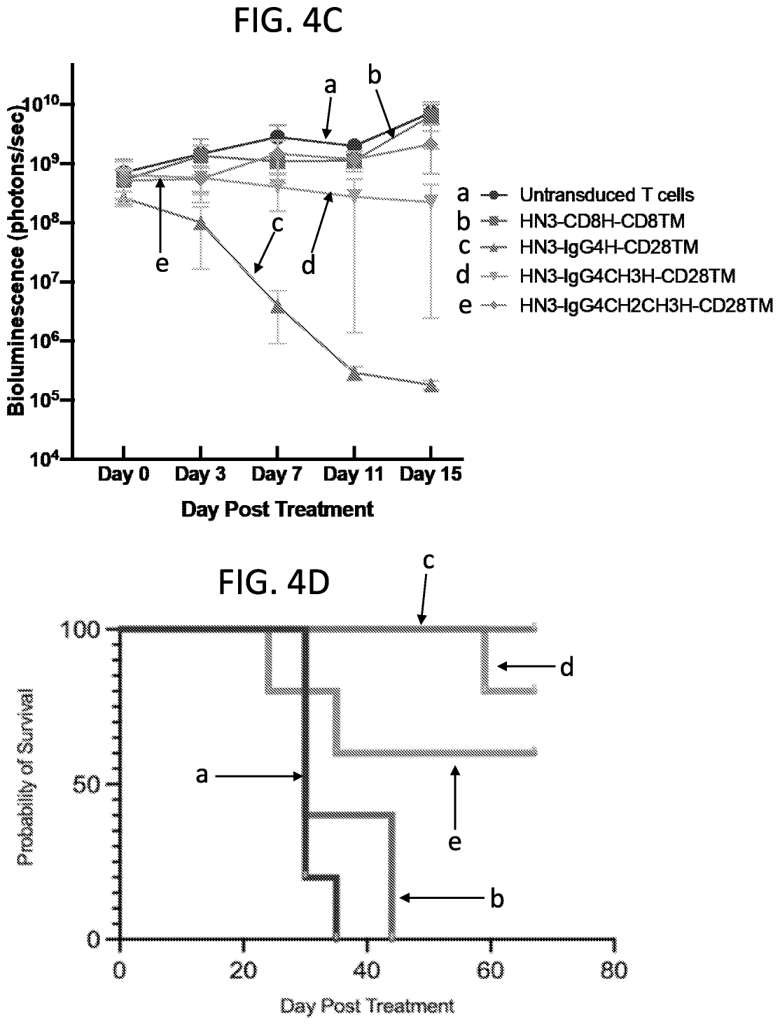

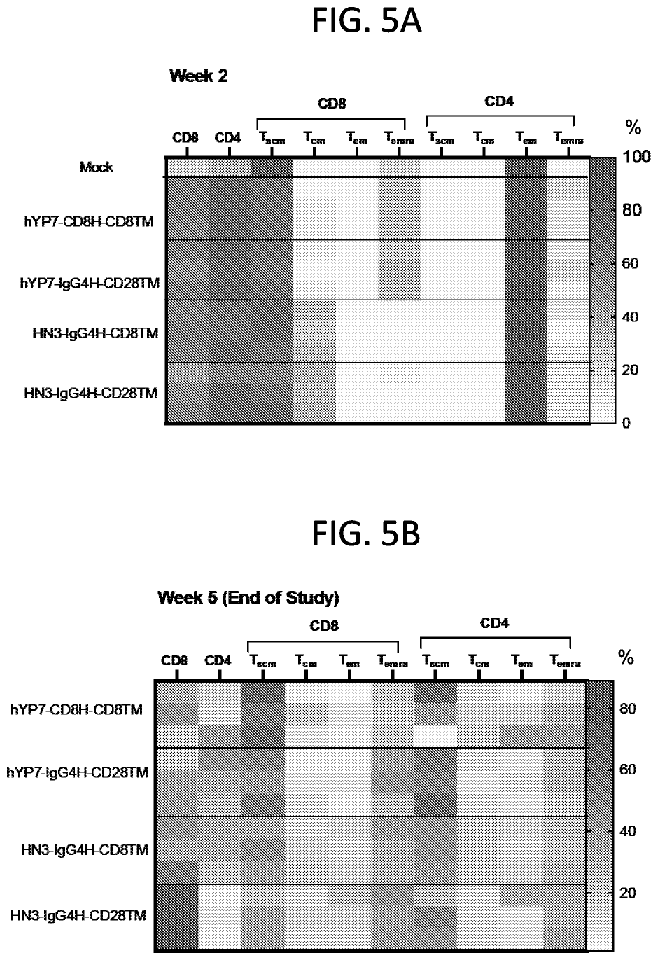

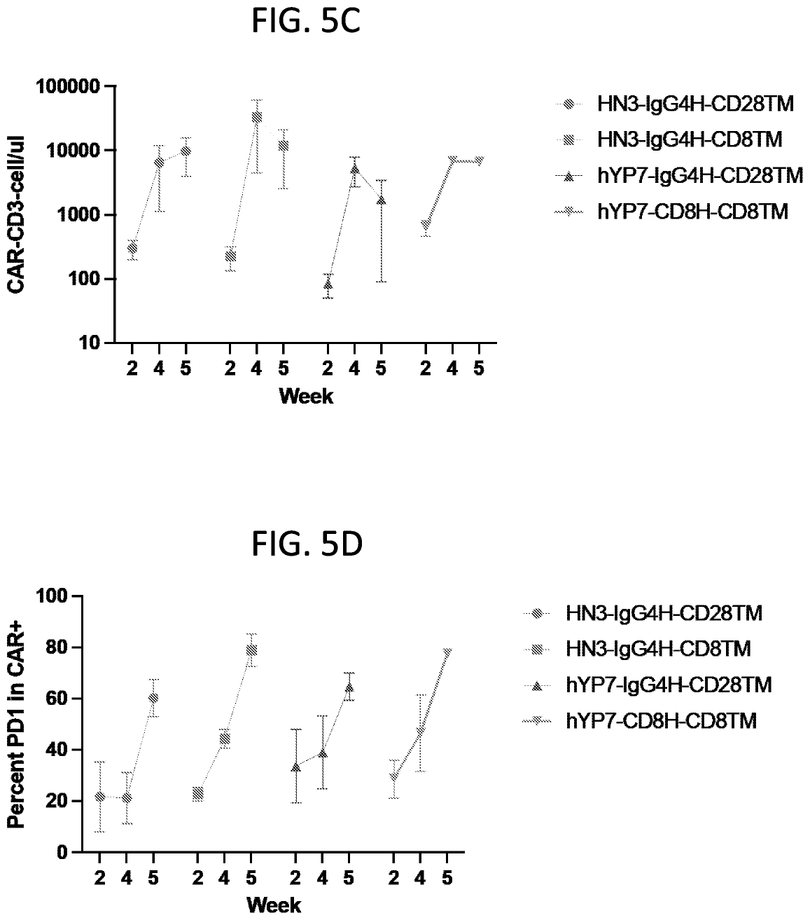

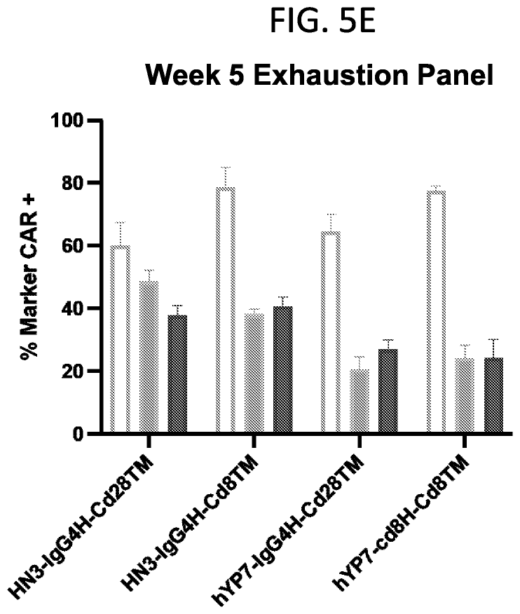

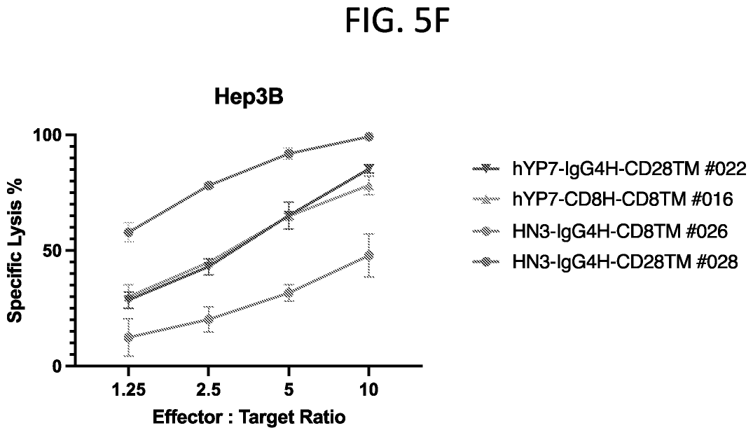

CAR T cells have transformed treatment of CD19+ B cell malignancies; however, their success in solid tumors is limited by several barriers, including the paucity of tumor-specific antigens, the inability of CAR T cells to efficiently expand at the tumor site, and heterogenous antigen expression (Kochenderfer et al., Blood. 2012; 119(12):2709-2720; Porter et al., N Eng J Med 2011;365(8):725-733; Jiang et al., Front Immunol 2017;7:690; Gao et al., Clin Cancer Res 2014;20(24):6418-6428; Ishiguro et al., Sci Transl Med 2017;9(410)aal4291; Ishiguro et al., Cancer Res 2008;68(23):9832-9838; Losic et al., Nat Commun 2020;11(1):291. The present disclosure addresses these challenges using rational engineering strategies to precisely alter the structural components of CARs. Specifically, it is disclosed herein that immune cells expressing GPC3-targeted CARs were most effective when a short hinge sequence derived from IgG4 was used in the construct. In addition, combining an IgG4 hinge (IgG4H) with a CD28 transmembrane (CD28TM) domain produced the most potent anti-tumor effect (FIGS. 3A-3D and 4A-4D). The data disclosed herein also demonstrate that GPC3-targeted IgG4H-CD28TM CAR immune cells induce strong CD8 T cell and Temra responses (FIG. 5B) that lead to surprisingly swift and durable tumor eradication. Provided herein are CARs that include an extracellular antigen-binding domain that specifically binds GPC3; a wild-type or modified IgG4 hinge region; a transmembrane domain; an intracellular co-stimulatory domain; and an intracellular signaling domain. In some aspects, the hinge region includes or consists of the modified IgG4 hinge sequence ESKYGPPCPPCP (SEQ ID NO: 43). In other aspects, the hinge region includes or consists of the wild-type IgG4 sequence ESKYGPPCPSCP (SEQ ID NO: 52). In some aspects, the transmembrane domain includes a CD28 transmembrane domain or a CD8 transmembrane domain.

In some aspects, the antigen-binding domain of the CAR specifically binds GPC3 with high affinity. In some examples, the antigen-binding domain includes a GPC3-specific single-domain antibody or a GPC3-specific scFv. In some examples, the antigen-binding domain includes one or more CDR sequences (such as one, two or all three CDR sequences) from GPC3-specific single-domain antibody HN3. In other examples, the antigen-binding domain includes one or more CDR sequences (such as one, two, three, four, five or all six CDR sequences) from GPC3-specific monoclonal antibody hYP7, YP9.1, YP8, YP9, YP6, YP7 or HS20.

In some aspects, the antigen-binding domain of the CAR is a single-domain antibody that includes the CDR1, CDR2 and CDR3 sequences of SEQ ID NO: 18 (HN3). In some examples, the CDR1, CDR2 and CDR3 sequences respectively comprise residues 31-35, 50-65 and 96-105 of SEQ ID NO: 18; or residues 26-33, 51-57 and 96-105 of SEQ ID NO: 18. In specific examples, the amino acid sequence of the single-domain antibody is at least 80%, at least 85%, at least 90%, at least 95%, at least 96%, at least 97%, at least 98%, or at least 99% identical to SEQ ID NO: 18 (and includes the CDR1, CDR2 and CDR3 sequences of SEQ ID NO: 18). In particular non-limiting examples, the amino acid sequence of the single-domain antibody comprises or consists of SEQ ID NO: 18.

In other aspects, the antigen-binding domain of the CAR includes a variable heavy (VH) domain and a variable light (VL) domain and the VH domain includes the CDR1, CDR2 and CDR3 sequences of SEQ ID NO: 20 (hYP7 VH domain), and the VL domain includes the CDR1, CDR2 and CDR3 sequences of SEQ ID NO: 22 (hYP7 VL domain). In some examples, the VH domain CDR1, CDR2 and CDR3 sequences respectively comprise residues 31-35, 50-68 and 101-106 of SEQ ID NO: 20; or residues 26-33, 51-60 and 99-106 of SEQ ID NO: 20. In some examples, the VL domain CDR1, CDR2 and CDR3 sequences respectively comprise residues 24-40, 56-62 and 95-103 of SEQ ID NO: 22; or residues 27-38, 56-58 and 95-103 of SEQ ID NO: 22. In specific examples, the amino acid sequence of the VH domain is at least 80%, at least 85%, at least 90%, at least 95%, at least 96%, at least 97%, at least 98%, or at least 99% identical to SEQ ID NO: 20 (and includes the CDR1, CDR2 and CDR3 sequences of SEQ ID NO: 20); and/or the amino acid sequence of the VL domain is at least 80%, at least 85%, at least 90%, at least 95%, at least 96%, at least 97%, at least 98%, or at least 99% identical to SEQ ID NO: 22 (and includes the CDR1, CDR2 and CDR3 sequences of SEQ ID NO: 22). In particular non-limiting examples, the amino acid sequence of the VH domain comprises or consists of SEQ ID NO: 20; and/or the amino acid sequence of the VL domain comprises or consists of SEQ ID NO: 22. In other particular examples, the antigen-binding domain is a scFv that includes the amino acid sequence of residues 1-245 of SEQ ID NO: 14.

In other aspects, the antigen-binding domain of the CAR includes a VH domain and a VL domain and the VH domain includes the CDR1, CDR2 and CDR3 sequences of SEQ ID NO: 24 (YP9.1 VH domain), and the VL domain includes the CDR1, CDR2 and CDR3 sequences of SEQ ID NO: 26 (YP9.1 VL domain). In some examples, the VH domain CDR1, CDR2 and CDR3 sequences respectively comprise residues 31-35, 50-68 and 101-106 of SEQ ID NO: 24; or residues 26-33, 51-60 and 99-106 of SEQ ID NO: 24. In some examples, the VL domain CDR1, CDR2 and CDR3 sequences respectively comprise residues 24-40, 56-62 and 95-103 of SEQ ID NO: 26; or residues 27-38, 56-58 and 95-103 of SEQ ID NO: 26. In specific examples, the amino acid sequence of the VH domain is at least 80%, at least 85%, at least 90%, at least 95%, at least 96%, at least 97%, at least 98%, or at least 99% identical to SEQ ID NO: 24 (and includes the CDR1, CDR2 and CDR3 sequences of SEQ ID NO: 24); and/or the amino acid sequence of the VL domain is at least 80%, at least 85%, at least 90%, at least 95%, at least 96%, at least 97%, at least 98%, or at least 99% identical to SEQ ID NO: 26 (and includes the CDR1, CDR2 and CDR3 sequences of SEQ ID NO: 26). In particular non-limiting examples, the amino acid sequence of the VH domain comprises or consists of SEQ ID NO: 24; and/or the amino acid sequence of the VL domain comprises or consists of SEQ ID NO: 26.

In other aspects, the antigen-binding domain of the CAR includes a VH domain and a VL domain and the VH domain includes the CDR1, CDR2 and CDR3 sequences of SEQ ID NO: 28 (YP8 VH domain), and the VL domain includes the CDR1, CDR2 and CDR3 sequences of SEQ ID NO: 30 (YP8 VL domain). In some examples, the VH domain CDR1, CDR2 and CDR3 sequences respectively comprise residues 31-35, 50-68 and 101-106 of SEQ ID NO: 28; or residues 26-33, 51-60 and 99-106 of SEQ ID NO: 28. In some examples, the VL domain CDR1, CDR2 and CDR3 sequences respectively comprise residues 24-40, 56-62 and 95-103 of SEQ ID NO: 30; or residues 27-38, 56-58 and 95-103 of SEQ ID NO: 30. In specific examples, the amino acid sequence of the VH domain is at least 80%, at least 85%, at least 90%, at least 95%, at least 96%, at least 97%, at least 98%, or at least 99% identical to SEQ ID NO: 28 (and includes the CDR1, CDR2 and CDR3 sequences of SEQ ID NO: 28); and/or the amino acid sequence of the VL domain is at least 80%, at least 85%, at least 90%, at least 95%, at least 96%, at least 97%, at least 98%, or at least 99% identical to SEQ ID NO: 30 (and includes the CDR1, CDR2 and CDR3 sequences of SEQ ID NO: 30). In particular non-limiting examples, the amino acid sequence of the VH domain comprises or consists of SEQ ID NO: 28; and/or the amino acid sequence of the VL domain comprises or consists of SEQ ID NO: 30.

In other aspects, the antigen-binding domain of the CAR includes a VH domain and a VL domain and the VH domain includes the CDR1, CDR2 and CDR3 sequences of SEQ ID NO: 58 (YP6 VH domain), and the VL domain includes the CDR1, CDR2 and CDR3 sequences of SEQ ID NO: 30 (YP6 VL domain). In some examples, the VH domain CDR1, CDR2 and CDR3 sequences respectively comprise residues 31-35, 50-68 and 101-106 of SEQ ID NO: 58; or residues 26-33, 51-60 and 99-106 of SEQ ID NO: 28. In some examples, the VL domain CDR1, CDR2 and CDR3 sequences respectively comprise residues 24-40, 56-62 and 95-103 of SEQ ID NO: 30; or residues 27-38, 56-58 and 95-103 of SEQ ID NO: 30. In specific examples, the amino acid sequence of the VH domain is at least 80%, at least 85%, at least 90%, at least 95%, at least 96%, at least 97%, at least 98%, or at least 99% identical to SEQ ID NO: 58 (and includes the CDR1, CDR2 and CDR3 sequences of SEQ ID NO: 58); and/or the amino acid sequence of the VL domain is at least 80%, at least 85%, at least 90%, at least 95%, at least 96%, at least 97%, at least 98%, or at least 99% identical to SEQ ID NO: 30 (and includes the CDR1, CDR2 and CDR3 sequences of SEQ ID NO: 30). In particular non-limiting examples, the amino acid sequence of the VH domain comprises or consists of SEQ ID NO: 58; and/or the amino acid sequence of the VL domain comprises or consists of SEQ ID NO: 30.

In other aspects, the antigen-binding domain of the CAR includes a VH domain and a VL domain and the VH domain includes the CDR1, CDR2 and CDR3 sequences of SEQ ID NO: 32 (YP9 VH domain), and the VL domain includes the CDR1, CDR2 and CDR3 sequences of SEQ ID NO: 34, 36 or 38 (YP9 clone 9, YP9 clone 10 and YP9 clone 1 VL domain). In some examples, the VH domain CDR1, CDR2 and CDR3 sequences respectively comprise residues 31-35, 50-68 and 101-106 of SEQ ID NO: 32; or residues 26-33, 51-60 and 99-106 of SEQ ID NO: 32. In some examples, the VL domain CDR1, CDR2 and CDR3 sequences respectively comprise residues 24-40, 56-62 and 95-103 of SEQ ID NO: 34, 36 or 38; or residues 27-38, 56-58 and 95-103 of SEQ ID NO: 34, 36 or 38. In specific examples, the amino acid sequence of the VH domain is at least 80%, at least 85%, at least 90%, at least 95%, at least 96%, at least 97%, at least 98%, or at least 99% identical to SEQ ID NO: 32 (and includes the CDR1, CDR2 and CDR3 sequences of SEQ ID NO: 32); and/or the amino acid sequence of the VL domain is at least 80%, at least 85%, at least 90%, at least 95%, at least 96%, at least 97%, at least 98%, or at least 99% identical to SEQ ID NO: 34, 36 or 38 (and includes the CDR1, CDR2 and CDR3 sequences of SEQ ID NO: 34, 36 or 38, respectively). In particular non-limiting examples, the amino acid sequence of the VH domain comprises or consists of SEQ ID NO: 32; and/or the amino acid sequence of the VL domain comprises or consists of SEQ ID NO: 34, 36 or 38.

In other aspects, the antigen-binding domain of the CAR includes a variable heavy (VH) domain and a variable light (VL) domain and the VH domain includes the CDR1, CDR2 and CDR3 sequences of SEQ ID NO: 54 (YP7 VH domain), and the VL domain includes the CDR1, CDR2 and CDR3 sequences of SEQ ID NO: 56 (YP7 VL domain). In some examples, the VH domain CDR1, CDR2 and CDR3 sequences respectively comprise residues 31-35, 50-68 and 101-106 of SEQ ID NO: 54; or residues 26-33, 51-60 and 99-106 of SEQ ID NO: 54. In some examples, the VL domain CDR1, CDR2 and CDR3 sequences respectively comprise residues 24-40, 56-62 and 95-103 of SEQ ID NO: 56; or residues 27-38, 56-58 and 95-103 of SEQ ID NO: 56. In specific examples, the amino acid sequence of the VH domain is at least 80%, at least 85%, at least 90%, at least 95%, at least 96%, at least 97%, at least 98%, or at least 99% identical to SEQ ID NO: 54 (and includes the CDR1, CDR2 and CDR3 sequences of SEQ ID NO: 54); and/or the amino acid sequence of the VL domain is at least 80%, at least 85%, at least 90%, at least 95%, at least 96%, at least 97%, at least 98%, or at least 99% identical to SEQ ID NO: 56 (and includes the CDR1, CDR2 and CDR3 sequences of SEQ ID NO: 56). In particular non-limiting examples, the amino acid sequence of the VH domain comprises or consists of SEQ ID NO: 54; and/or the amino acid sequence of the VL domain comprises or consists of SEQ ID NO: 56.

In other aspects, the antigen-binding domain of the CAR includes a VH domain and a VL domain and the VH domain includes the CDR1, CDR2 and CDR3 sequences of SEQ ID NO: 40 (HS20 VH domain), and the VL domain includes the CDR1, CDR2 and CDR3 sequences of SEQ ID NO: 42 (HS20 VL domain). In some examples, the VH domain CDR1, CDR2 and CDR3 sequences respectively comprise residues 31-35, 50-66 and 97-105 of SEQ ID NO: 40; or residues 26-33, 51-58 and97-105 of SEQ ID NO: 40. In some examples, the VL domain CDR1, CDR2 and CDR3 sequences respectively comprise residues 24-34, 50-56 and 89-97 of SEQ ID NO: 42; or residues 27-32, 50-52 and 89-97 of SEQ ID NO: 42. In specific examples, the amino acid sequence of the VH domain is at least 80%, at least 85%, at least 90%, at least 95%, at least 96%, at least 97%, at least 98%, or at least 99% identical to SEQ ID NO: 40 (and includes the CDR1, CDR2 and CDR3 sequences of SEQ ID NO: 40); and/or the amino acid sequence of the VL domain is at least 80%, at least 85%, at least 90%, at least 95%, at least 96%, at least 97%, at least 98%, or at least 99% identical to SEQ ID NO: 42 (and includes the CDR1, CDR2 and CDR3 sequences of SEQ ID NO: 42). In particular non-limiting examples, the amino acid sequence of the VH domain comprises or consists of SEQ ID NO: 40; and/or the amino acid sequence of the VL domain comprises or consists of SEQ ID NO: 42.

In some aspects, the transmembrane domain of the CAR includes a CD28 transmembrane domain. In some examples, the amino acid sequence of the CD28 transmembrane domain is at least 80%, at least 85%, at least 90%, at least 95%, at least 96%, at least 97%, at least 98%, or at least 99% identical to SEQ ID NO: 46. In particular non-limiting examples, the amino acid sequence of the CD28 transmembrane domain includes or consists of SEQ ID NO: 46. In other aspects, the transmembrane domain of the CAR includes a CD8 transmembrane domain. In some examples, the amino acid sequence of the CD8 transmembrane domain is at least 80%, at least 85%, at least 90%, at least 95%, at least 96%, at least 97%, at least 98%, or at least 99% identical to SEQ ID NO: 47. In particular non-limiting examples, the amino acid sequence of the CD8 transmembrane domain includes or consists of SEQ ID NO: 47.

In some aspects, the co-stimulatory domain of the CAR includes a 4-1BB signaling moiety. In some examples, the amino acid sequence of the 4-1BB signaling moiety is at least 80%, at least 85%, at least 90%, at least 95%, at least 96%, at least 97%, at least 98%, or at least 99% identical to SEQ ID NO: 48. In particular non-limiting examples, the amino acid sequence of the 4-1BB signaling moiety includes or consists of SEQ ID NO: 48.

In some aspects, the signaling domain of the CAR includes a CD3ζ signaling domain. In some examples, the amino acid sequence of the signaling domain is at least 80%, at least 85%, at least 90%, at least 95%, at least 96%, at least 97%, at least 98%, or at least 99% identical to SEQ ID NO: 49. In particular non-limiting examples, the amino acid sequence of the CD33 signaling domain includes or consists of SEQ ID NO: 49.

In some aspects, the amino acid sequence of the CAR is at least 80%, at least 85%, at least 90%, at least 95%, at least 96%, at least 97%, at least 98%, or at least 99% identical to any one of SEQ ID NOs: 2, 4, 6, 8, 10, 12 and 14. In some examples, the amino acid sequence of the CAR comprises any one of SEQ ID NOs: 2, 4, 6, 8, 10, 12 and 14. In specific examples, the amino acid sequence includes SEQ ID NO: 6 (HN3-IgG4H-CD28TM) or SEQ ID NO: 14 (hYP7-IgG4H-CD28TM).

Further provided are nucleic acid molecules that encode a CAR disclosed herein. In some aspects, the nucleic acid molecule is operably linked to a promoter (such as an inducible or constitutive promoter). In some examples, the nucleic acid molecule is at least 80%, at least 85%, at least 90%, at least 95%, at least 96%, at least 97%, at least 98%, or at least 99% identical to any one of SEQ ID NOs: 1, 3, 5, 7, 9, 11 and 13. In particular examples, the nucleic acid molecule includes any one of SEQ ID NOs: 1, 3, 5, 7, 9, 11 and 13. In specific non-limiting examples, the nucleic acid molecule includes SEQ ID NO: 5 (HN3-IgG4H-CD28TM) or SEQ ID NO: 13 (hYP7-IgG4H-CD28TM).

In some aspects, the nucleic acid molecule includes, in the 5′ to 3′ direction, a nucleic acid encoding a first granulocyte-macrophage colony stimulating factor receptor signal sequence (GMCSFRss); a nucleic acid encoding the antigen-binding domain; a nucleic acid encoding the IgG4 hinge region; a nucleic acid encoding the transmembrane domain; a nucleic acid encoding the co-stimulatory domain; a nucleic acid encoding the signaling domain; a nucleic acid encoding a self-cleaving 2A peptide; a nucleic acid encoding a second GMCSFRss; and a nucleic acid encoding a truncated human epidermal growth factor receptor (huEGFRt). In some examples, the nucleic acid molecule further includes a human elongation factor 1α (EF1α) promoter sequence 5′ of the nucleic acid encoding the first GMCSFRss (see WO 2019/094482).

Vectors that include a nucleic acid molecule disclosed herein are further provided. In some examples, the vector is a viral vector, such as a lentiviral vector, an adenovirus vector or an adeno-associated virus vector.

Also provided are isolated that cells that include a nucleic acid molecule encoding a CAR disclosed herein and/or express a CAR disclosed herein. In some aspects, the cell is an immune cell, such as a T cell, B cell, NK cell or macrophage. In other aspects, the cell is an induced pluripotent stem cell (iPSC).

Further provided are compositions that include a pharmaceutically acceptable carrier (such as water or saline) and a CAR, nucleic acid molecule, vector, or cell disclosed herein. In some examples the composition is frozen. In some examples the composition is frozen and includes cells and DMSO or other cryopreservative. In some examples the composition is lyophilized.

Methods of treating a GPC3-positive cancer, or inhibiting tumor growth or metastasis of a GPC3-positive cancer, in a subject are also provided. In some aspects, the methods include administering to the subject a therapeutically effective amount of a CAR, nucleic acid molecule, vector, cell or composition disclosed herein. In some examples, the GPC3-positive cancer is a solid tumor. In particular non-limiting examples, the GPC3-positive cancer is a hepatocellular carcinoma (HCC), melanoma, ovarian clear-cell carcinoma, yolk sac tumor (YST), neuroblastoma, hepatoblastoma, Wilms' tumor, squamous cell carcinoma of the lung, testicular nonseminomatous germ cell tumor, liposarcoma, cervical intraepithelial neoplasia, adenoma of the adrenal gland, schwannoma or embryonal tumor. In specific examples, the GPC3-positive cancer is HCC.

### IV. GPC3-Specific Antibody Sequences

The CARs disclosed herein include an antibody (or antigen-binding fragment thereof) that specifically binds GPC3. In some aspects, the antibody is HN3, a human single-domain (VH) monoclonal antibody, or hYP7, a humanized mouse antibody, in scFv format. In other aspects, the antibody is YP9, YP8, YP6, YP7 or YP9.1 (such as in scFv format), or humanized versions thereof. In yet other aspects, the antibody is HS20 (such as in scFv format), which is a human antibody. The HN3, hYP7, YP9, YP8, YP6, YP7, YP9.1 and HS20 antibodies are described in PCT Publication Nos. WO 2012/145469, WO 2012/145469 and WO 2019/094482. The nucleotide and amino acid sequences of HN3, hYP7, YP9, YP8, YP6, YP7, YP9.1 and HS20 are provided below. Tables 1-13 list the amino acid positions of the CDR1, CDR2 and CDR3 of HN3, and the CDR1, CDR2 and CDR3 of the VH and VL domains of hYP7, YP9, YP8, YP6, YP7, YP9.1 and HS20, as determined using Kabat and IMGT. One could readily determine the CDR boundaries using an alternative numbering scheme, such as the Paratome or Chothia numbering schemes.

HN3 DNA Sequence
(SEQ ID NO: 17)
CAGGTGCAGCTGGTGCAGTCTGGGGGAGGCTTGGTACAGCCTGGAGGGTC
CCTGAGACTCTCCTGTGCAGCCTCTTATTTCGATTTCGATTCTTATGAAA
TGAGCTGGGTCCGCCAGGCTCCAGGGAAGGGCCTAGAGTGGATTGGGAGT
ATCTATCATAGTGGGAGCACCTACTACAACCCGTCCCTCAAGAGTCGAGT
CACCATCTCCAGAGACAATTCCAAGAACACGCTGTATCTGCAAATGAACA
CCCTGAGAGCCGAGGACACAGCCACGTATTACTGTGCGAGAGTAAATATG
GACCGATTTGACTACTGGGGCCAGGGAACCCTGGTCACCGTCTCCTCA
HN3 Protein Sequence
(SEQ ID NO: 18)
QVQLVQSGGGLVQPGGSLRLSCAASYFDFDSYEMSWVRQAPGKGLEWIGS
IYHSGSTYYNPSLKSRVTISRDNSKNTLYLQMNTLRAEDTATYYCARVNM
DRFDYWGQGTLVTVSS

TABLE 1
Locations of the CDRs in the HN3 Sequence (SEQ ID NO: 18)

| CDR | Kabat | IMGT |
|---|---|---|
| CDR1 | amino acids 31-35 | amino acids 26-33 |
| CDR2 | amino acids 50-65 | amino acids 51-57 |
| CDR3 | amino acids 96-105 | amino acids 96-105 |

hYP7 VH DNA Sequence
(SEQ ID NO: 19)
GAGGTGCAGCTTGTTGAGTCTGGTGGAGGATTGGTGCAGCCTGGAGGGTC
ATTGAGACTCTCATGTGCAGCCTCTGGATTCACCTTCAATAAGAATGCCA
TGAATTGGGTCCGCCAGGCTCCAGGAAAGGGTTTGGAATGGGTTGGCCGC
ATAAGAAATAAAACTAATAATTATGCAACATATTATGCCGATTCAGTGAA
AGCCAGGTTTACCATCTCCAGAGATGATTCAAAGAACTCACTCTATCTGC
AAATGAACAGCTTGAAAACCGAGGACACAGCCGTGTACTATTGTGTGGCT
GGTAACTCGTTTGCTTACTGGGGCCAAGGGACTCTGGTCACTGTCTCTGC
A
hYP7 VH Protein Sequence
(SEQ ID NO: 20)
EVQLVESGGGLVQPGGSLRLSCAASGFTFNKNAMNWVRQAPGKGLEWVGR
IRNKTNNYATYYADSVKARFTISRDDSKNSLYLQMNSLKTEDTAVYYCVA
GNSFAYWGQGTLVTVSA
hYP7 VL DNA Sequence
(SEQ ID NO: 21)
GACATTGTGATGACCCAGTCTCCAGACTCCCTAGCTGTGTCACTGGGAGA
GAGGGCCACTATCAACTGCAAGTCCAGTCAGAGCCTTTTATATAGCAGCA
ATCAAAAGAACTACTTGGCCTGGTACCAACAGAAACCAGGGCAGCCTCCT
AAACTGCTGATTTACTGGGCATCCAGTAGGGAATCTGGGGTCCCTGATCG
CTTCAGTGGCAGTGGATCTGGGACAGATTTCACTCTCACCATCAGCAGTC
TGCAGGCTGAAGACGTGGCAGTTTATTACTGTCAGCAATATTATAACTAT
CCGCTCACGTTCGGTCAGGGGACCAAGTTGGAGATCAAA
hYP7 VL Protein Sequence
(SEQ ID NO: 22)
DIVMTQSPDSLAVSLGERATINCKSSQSLLYSSNQKNYLAWYQQKPGQPP
KLLIYWASSRESGVPDRFSGSGSGTDFTLTISSLQAEDVAVYYCQQYYNY
PLTFGQGTKLEIK

TABLE 2
Locations of the CDRs in the hYP7
VH Sequence (SEQ ID NO: 20)

| CDR | Kabat | IMGT |
|---|---|---|
| CDR1 | amino acids 31-35 | amino acids 26-33 |
| CDR2 | amino acids 50-68 | amino acids 51-60 |
| CDR3 | amino acids 101-106 | amino acids 99-106 |

TABLE 3
Locations of the CDRs in the hYP7
VL Sequence (SEQ ID NO: 22)

| CDR | Kabat | IMGT |
|---|---|---|
| CDR1 | amino acids 24-40 | amino acids 27-38 |
| CDR2 | amino acids 56-62 | amino acids 56-58 |
| CDR3 | amino acids 95-103 | amino acids 95-103 |

YP9.1 VH DNA Sequence
(SEQ ID NO: 23)
GAGGTGCAGCTTGTTGAGACTGGCGGAGGATTGGTGCAGCCTGAAGGGTC
ATTGAAACTCTCATGTGCAACGTCTGGATTCAACTTCAATACCAATGCCA
TGAACTGGGTCCGCCAGGCTCCAGGAAAGGGTTTGGAATGGGTTGCTCGC
GTAAGAAATAAAACTAATAATTATGCAACATATTATGCCGATTCAGTGAA
AGACAGGTTCACCATCTCCAGAGATGATTCACAAAGAATGGTCTTTCTGC
AAATGAATAACTTGAAAACTGAGGACACAGCCATCTATTACTGTGTGGCG
GGGAACTCGTTTGCTTATTGGGGCCAAGGGACTCTGGTCACTGTCTCTCC
T
YP9.1 VH Protein Sequence
(SEQ ID NO: 24)
EVQLVETGGGLVQPEGSLKLSCATSGFNFNTNAMNWVRQAPGKGLEWVAR
VRNKTNNYATYYADSVKDRFTISRDDSQRMVFLQMNNLKTEDTAIYYCVA
GNSFAYWGQGTLVTVSP
YP9.1 VL DNA Sequence
(SEQ ID NO: 25)
GACATTGTGATGTCACAGTCTCCATCCTCCCTAGCTGTGTCAGTTGGAGA
GAGGGTTACTATGAACTGCAAGTCCAGTCAGAGTCTTTTATATAGTAGCA
ATCAAAAGAACTACTTGGCCTGGTACCAGCAGAAACCAGGGCAGTCTCCT
AAACTGCTGATTTACTGGGCATCCACTAGGGAATCTGGGGTCCCTGATCG
CTTCACAGGCAGTGTATCTGGGACAGATTTCACTCTCACCATCAGCAGTG
TGAAGGCTGAAGACCTGGCAGTTTATTACTGTCAGCAATATTATAACTAT
CCGCTCACGTTCGGTGCTGGGACCAAGCTGGAGCTGAAA
YP9.1 VL Protein Sequence
(SEQ ID NO: 26)
DIVMSQSPSSLAVSVGERVTMNCKSSQSLLYSSNQKNYLAWYQQKPGQSP
KLLIYWASTRESGVPDRFTGSVSGTDFTLTISSVKAEDLAVYYCQQYYNY
PLTFGAGTKLELK

TABLE 4
Locations of the CDRs in the YP9.1
VH Sequence (SEQ ID NO: 24)

| CDR | Kabat | IMGT |
|---|---|---|
| CDR1 | amino acids 31-35 | amino acids 26-33 |
| CDR2 | amino acids 50-68 | amino acids 51-60 |
| CDR3 | amino acids 101-106 | amino acids 99-106 |

TABLE 5
Locations of the CDRs in the YP9.1
VL Sequence (SEQ ID NO: 26)

| CDR | Kabat | IMGT |
|---|---|---|
| CDR1 | amino acids 24-40 | amino acids 27-38 |
| CDR2 | amino acids 56-62 | amino acids 56-58 |
| CDR3 | amino acids 95-103 | amino acids 95-103 |

YP8 VH DNA Sequence
(SEQ ID NO: 27)
GAGGTGCAGCTTGTTGGAAGTGGTGGAGGATTGGTGCAGCCTGAAGGGTC
ATTGAAACTCTCATGTGCAGCCTCTGGATTCACCTTCAAGACCAATGCCA
TGAACTGGGTCCGCCAGGCTCCAGGAAAGGGTTTGGAATGGGTTGCTCGC
ATAAGAAATAAAACTAATAATTATGCAACATATTATGCCGACTCAGTGAA
AGACAGGTTCACCATCTCCAGAGATGATTCACAAAGCATGCTCTATCTGC
AAATGAACAACTTGAAAACTGAAGACACAGCCATGTATTTCTGTGTGGCC
GGTAACTCGTTTGCTTACTGGGGCCAGGGGACTCTGGTCACTGTCTCTGC
A
YP8 VH Protein Sequence
(SEQ ID NO: 28)
EVQLVGSGGGLVQPEGSLKLSCAASGFTFKTNAMNWVRQAPGKGLEWVAR
IRNKTNNYATYYADSVKDRFTISRDDSQSMLYLQMNNLKTEDTAMYFCVA
GNSFAYWGQGTLVTVSA
YP8 VL DNA Sequence
(SEQ ID NO: 29)
GACATTGTGATGTCACAGTCTCCATCCTCCCTAGCTGTGTCAGTTGGAGA
GAAGGTTACTGTGAACTGCAAGTCCAGTCAGAGCCTTTTATATAGTAACA
ATCAAAAGAACTACTTGGCCTGGTACCAGCAGAAACCAGGGCAGTCTCCT
AAACTGCTGATTTACTGGGCATCAACTAGGGAATATGGGGTCCCTGATCG
CTTCACAGGCAGTGGATCTGGGACAGATTTCACTCTCACCATCAGCAGTG
TGAAGGCTGAAGACCTGGCAGTTTATTACTGTCAGCAATATTATAACTAT
CCGCTCACGTTCGGTGCTGGGACCAAGCTGGAGCTGAAA
YP8 VL Protein Sequence
(SEQ ID NO: 30)
DIVMSQSPSSLAVSVGEKVTVNCKSSQSLLYSNNQKNYLAWYQQKPGQSP
KLLIYWASTREYGVPDRFTGSGSGTDFTLTISSVKAEDLAVYYCQQYYNY
PLTFGAGTKLELK
YP6 VH DNA Sequence
(SEQ ID NO: 57)
GAGGTGCAGCTTGTTGGAAGTGGTGGAGGATTGGTGCAGCCTGAAGGGTC
ATTGAAACTCTCATGTGCAGCCTCTGGATTCACCTTCAAGACCAATGCCA
TGAACTGGGTCCGCCAGGCTCCAGGAAAGGGTTTGGAATGGGTTGCTCGC
ATAAGAAATAAAACTAATAATTATGCAACATATTATGCCGACTCAGTGAA
AGACAGGTTCACCATCTCCAGAGATGATTCACAAAGCATGCTCTATCTGC
AAATGAACAACTTGAAAACTGAAGACACAGCCATGTATTTCTGTGTGGCC
GGTAACTCGTTTGCTTACTTGGGCCAGGGGACTCTGGTCACTGTCTCTGC
A
YP6 VH Protein Sequence
(SEQ ID NO: 58)
EVQLVGSGGGLVQPEGSLKLSCAASGFTFKTNAMNWVRQAPGKGLEWVAR
IRNKTNNYATYYADSVKDRFTISRDDSQSMLYLQMNNLKTEDTAMYFCVA
GNSFAYLGQGTLVTVSA

TABLE 6
Locations of the CDRs in the YP8 & YP6
VH Sequences (SEQ ID NOs: 28 & 58)*

| CDR | Kabat | IMGT |
|---|---|---|
| CDR1 | amino acids 31-35 | amino acids 26-33 |
| CDR2 | amino acids 50-68 | amino acids 51-60 |
| CDR3 | amino acids 101-106 | amino acids 99-106 |

TABLE 7
Locations of the CDRs in the YP8 &
YP6 VL Sequence (SEQ ID NO: 30)*

| CDR | Kabat | IMGT |
|---|---|---|
| CDR1 | amino acids 24-40 | amino acids 27-38 |
| CDR2 | amino acids 56-62 | amino acids 56-58 |
| CDR3 | amino acids 95-103 | amino acids 95-103 |

*YP6 and YP8 have the same VL sequence; the YP6 VH sequence differs from the YP8 VH sequence by a single amino acid residue immediately following CDR3 (a tryptophan to leucine change).

YP9 VH DNA Sequence
(SEQ ID NO: 31)
GAGGTCCAGCTTGTTGAGACTGGTGGAGGATTGGTGCAGCCTAAAGGGTC
ATTGAAACTCTCATGTGCAGCCTCTGGATTCACCTTCAATACCAATGCCA
TGAACTGGGTCCGCCAGGCTCCAGGAAAGGGTTTGCAATGGGTTGCTCGC
GTAAGAAATAAAACTAATAATTATGCAACATATTATGCCGATTCCGTGAA
AGACAGGTTCACCATCTCCAGAGATGATTCACAAAGCATGCTCTATCTGC
AAATGAACAACTTGAAAACTGAAGACACGGCCATTTATTACTGTGTGGGG
GGTAACTCGTTTGCTTACTGGGGCCAAGGGACTCTGGTCACTGTCTCTGC
A
YP9 VH Protein Sequence
(SEQ ID NO: 32)
EVQLVETGGGLVQPKGSLKLSCAASGFTFNTNAMNWVRQAPGKGLQWVAR
VRNKTNNYATYYADSVKDRFTISRDDSQSMLYLQMNNLKTEDTAIYYCVG
GNSFAYWGQGTLVTVSA
YP9 clone 9 VL DNA Sequence
(SEQ ID NO: 33)
GACATTGTGATGTCCCAGTCTCCATCCTCCCTAGCTGTGTCAGTTGGAGA
GAAGGTTACTATGAGCTGCAAGTCCAGTCAGAGCCTTTTATATAGTAACA
ATCAAAAGAACTACTTGGCCTGGTACCACCAGAAACCAGGGCAGTCTCCT
AAACTGCTGATTTACTGGGCATCCACTAGGGAATCTGGGGTCCCTGATCG
CTTCACAGGCAGTGGATCTGGGACAGATTTCACTCTCACCATCAGCAGTG
TGAAGGCTGAAGACCTGGCAGTTTATTACTGTCAGCAATATTATAGCTAT
CCACTCACGTTCGGTGCTGGGACCAAGCTGGAGCTGAAA
YP9 clone 9 VL Protein Sequence
(SEQ ID NO: 34)
DIVMSQSPSSLAVSVGEKVTMSCKSSQSLLYSNNQKNYLAWYHQKPGQSP
KLLIYWASTRESGVPDRFTGSGSGTDFTLTISSVKAEDLAVYYCQQYYSY
PLTFGAGTKLELK
YP9 clone 10 VL DNA Sequence
(SEQ ID NO: 35)
GACATTGTGATGTCCCAGTCTCCATCCTCCCTAGCTGTGTCAGTTGGAGA
GAAGGTTACTATGAGCTGCAAGTCCAGTCAGAGCCTTTTATATAGTAACA
ATCAAAAGAACTACTTGGCCTGGTACCACCAGAAACCAGGGCAGTCTCCT
AAACTGCTGATTTACTGGGCATCCACTAGGGAATCTGGGGTCCCTGATCG
CTTCACAGGCAGTGGATCTGGGACAGATTTCACTCTCACCATCAGCAGTG
TGAAGGCTGAAGACCTGGCAGTTTATTACTGTCAGCAATATTATATCTAT
CCACTCACGTTCGGTGCTGGGACCAAGCTGGAGCTGAAA
YP9 clone 10 VL Protein Sequence
(SEQ ID NO: 36)
DIVMSQSPSSLAVSVGEKVTMSCKSSQSLLYSNNQKNYLAWYHQKPGQSP
KLLIYWASTRESGVPDRFTGSGSGTDFTLTISSVKAEDLAVYYCQQYYIY
PLTFGAGTKLELK
YP9 clone 1 VL DNA Sequence
(SEQ ID NO: 37)
GACATTGTGATGTCCCAGTCTCCATCCTCCCTAGCTGTGTCAGTTGGAGA
GAAGGTTACTATGAGCTGCAAGTCCAGTCAGAGCCTTTTATATAGTAACA
ATCAAAAGAACTACTTGGCCTGGTACCACCAGAAACCAGGGCAGTCTCCT
AAACTGCTGATTTACTGGGCATCCACTAGGGAATCTGGGGTCCCTGATCG
CTTCACAGGCAGTGGATCTGGGACAGATTTCACTCTCACCATCAGCAGTG
TGAAGGCTGAAGACCTGGCAGTTTATTACTGTCAGCAATATTATAGCTAT
CCACTCACGTTCGGTGCTGGGACCAAGCTGGAAATAAAA
YP9 clone 1 VL Protein Sequence
(SEQ ID NO: 38)
DIVMSQSPSSLAVSVGEKVTMSCKSSQSLLYSNNQKNYLAWYHQKPGQSP
KLLIYWASTRESGVPDRFTGSGSGTDFTLTISSVKAEDLAVYYCQQYYSY
PLTFGAGTKLEIK

TABLE 8
Locations of the CDRs in the YP9 VH Sequence (SEQ ID NO: 32)

| CDR | Kabat | IMGT |
|---|---|---|
| CDR1 | amino acids 31-35 | amino acids 26-33 |
| CDR2 | amino acids 50-68 | amino acids 51-60 |
| CDR3 | amino acids 101-106 | amino acids 99-106 |

TABLE 9
Locations of the CDRs in the YP9 VL
Sequence (SEQ ID NOs: 34, 36 and 38)

| CDR | Kabat | IMGT |
|---|---|---|
| CDR1 | amino acids 24-40 | amino acids 27-38 |
| CDR2 | amino acids 56-62 | amino acids 56-58 |
| CDR3 | amino acids 95-103 | amino acids 95-103 |

YP7 VH DNA Sequence
(SEQ ID NO: 53)
GAGGTGCAGCTTGTTGAGACTGGTGGAGGAATGGTGCAGCCTGAAGGGTC
ATTGAAACTCTCATGTGCAGCCTCTGGATTCACCTTCAATAAGAATGCCA
TGAATTGGGTCCGCCAGGCTCCAGGAAAGGGTTTGGAATGGGTTGCTCGC
ATAAGAAATAAAACTAATAATTATGCAACATATTATGCCGATTCAGTGAA
AGCCAGGTTTACCATCTCCAGAGATGATTCACAAAGCATGCTCTATCTGC
AAATGAACAACTTGAAAATTGAGGACACAGCCATGTACTATTGTGTGGCT
GGTAACTCGTTTGCTTACTGGGGCCAAGGGACTCTGGTCACTGTCTCTGC
A
YP7 VH Protein Sequence
(SEQ ID NO: 54)
EVQLVETGGGMVQPEGSLKLSCAASGFTFNKNAMNWVRQAPGKGLEWVAR
IRNKTNNYATYYADSVKARFTISRDDSQSMLYLQMNNLKIEDTAMYYCVA
GNSFAYWGQGTLVTVSA
YP7 VL DNA Sequence
(SEQ ID NO: 55)
GACATTGTGATGTCACAGTCTCCATCCTCCCTAGTTGTGTCAATTGGAGA
GAAGGTTACTATGACCTGCAAGTCCAGTCAGAGCCTTTTATATAGCAGCA
ATCAAAAGAACTACTTGGCCTGGTACCAACAGAAACCAGGGCAGTCTCCT
AAACTGCTGATTTACTGGGCATCCAGTAGGGAATCTGGGGTCCCTGATCG
CTTCACAGGCAGTGGATCTGGGACAGATTTCACTCTCACCATCAGCAGTG
TGAAGGCTGAAGACCTGGCAGTTTATTACTGTCAGCAATATTATAACTAT
CCGCTCACGTTCGGTGCTGGGACCAAGTTGGAGCTGAAA
YP7 VL Protein Sequence
(SEQ ID NO: 56)
DIVMSQSPSSLVVSIGEKVTMTCKSSQSLLYSSNQKNYLAWYQQKPGQSP
KLLIYWASSRESGVPDRFTGSGSGTDFTLTISSVKAEDLAVYYCQQYYNY
PLTFGAGTKLELK

TABLE 10
Locations of the CDRs in the YP7 VH Sequence (SEQ ID NO: 54)

| CDR | Kabat | IMGT |
|---|---|---|
| CDR1 | amino acids 31-35 | amino acids 26-33 |
| CDR2 | amino acids 50-68 | amino acids 51-60 |
| CDR3 | amino acids 101-106 | amino acids 99-106 |

TABLE 11
Locations of the CDRs in the YP7 VL Sequence (SEQ ID NO: 56)

| CDR | Kabat | IMGT |
|---|---|---|
| CDR1 | amino acids 24-40 | amino acids 27-38 |
| CDR2 | amino acids 56-62 | amino acids 56-58 |
| CDR3 | amino acids 95-103 | amino acids 95-103 |

HS20 VH DNA Sequence
(SEQ ID NO: 39)
GAGGTGCAGCTGTTGGAGTCTGGGGGAGGCTTGGTACAGCCTGGGGGGTC
CCTGAGACTCTCCTGTGCAGCCTCTGGATTCACCTTTAGCAGCTATGCCA
TGAGCTGGGTCCGCCAGGCTCCAGGGAAGGGGCTGGAGTGGGTCTCAACT
ATTCAGAAGCAGGGTCTGCCTACACAGTACGCAGACTCCGTGAAGGGGCG
GTTCACCATCTCCAGAGACAATTCCAAGAACACGCTGTATCTGCAAATGA
ACAGCCTGAGAGCCGAGGACACGGCCGTATATTACTGTGCGAAAAATCGG
GCTAAGTTTGACTACTGGGGCCAGGGAACCCTGGTCACCGTCTCGAGC
HS20 VH Protein Sequence
(SEQ ID NO: 40)
EVQLLESGGGLVQPGGSLRLSCAASGFTFSSYAMSWVRQAPGKGLEWVST
IQKQGLPTQYADSVKGRFTISRDNSKNTLYLQMNSLRAEDTAVYYCAKNR
AKFDYWGQGTLVTVSS
HS20 VL DNA Sequence
(SEQ ID NO: 41)
GACATCCAGATGACCCAGTCTCCATCCTCCCTGTCTGCATCTGTAGGAGA
CAGAGTCACCATCACTTGCCGGGCAAGTCAGAGCATTAGCAGCTATTTAA
ATTGGTATCAGCAGAAACCAGGGAAAGCCCCTAAGCTCCTGATCTATAAT
GCATCCATGTTGCAAAGTGGGGTCCCATCAAGGTTCAGTGGCAGTGGATC
TGGGACAGATTTCACTCTCACCATCAGCAGTCTGCAACCTGAAGATTTTG
CAACTTACTACTGTCAACAGAATCGGGGTTTTCCTCTGACGTTCGGCCAA
GGGACCAAGGTGGAAATCAAA
HS20 VL Protein Sequence
(SEQ ID NO: 42)
DIQMTQSPSSLSASVGDRVTITCRASQSISSYLNWYQQKPGKAPKLLIYN
ASMLQSGVPSRFSGSGSGTDFTLTISSLQPEDFATYYCQQNRGFPLTFGQ
GTKVEIK

TABLE 12
Locations of the CDRs in the HS20
VH Sequence (SEQ ID NO: 40)

| CDR | Kabat | IMGT |
|---|---|---|
| CDR1 | amino acids 31-35 | amino acids 26-33 |
| CDR2 | amino acids 50-66 | amino acids 51-58 |
| CDR3 | amino acids 97-105 | amino acids 97-105 |

TABLE 13
Locations of the CDRs in the HS20
VL Sequence (SEQ ID NO: 42)

| CDR | Kabat | IMGT |
|---|---|---|
| CDR1 | amino acids 24-34 | amino acids 27-32 |
| CDR2 | amino acids 50-56 | amino acids 50-52 |
| CDR3 | amino acids 89-97 | amino acids 89-97 |

### V. GPC3-Targeted CAR Sequences

The HN3 single-domain antibody and hYP7 scFv were used to generate several different CAR constructs utilizing either a CD8 hinge (SEQ ID NO: 45), IgG4 hinge (SEQ ID NO: 43), IgG4-CH3 hinge or IgG4-CH2-CH3 hinge, and either a CD8 transmembrane (TM) domain (SEQ ID NO: 47) or a CD28 transmembrane domain (SEQ ID NO: 46). In the CAR amino acid sequences provided below, the antigen-binding sequence (HN3 single-domain or hYP7 VH-linker-VL) is underlined, the hinge region (CD8α, IgG4, IgG4-CH3 or IgG4-CH2-CH3) is in bold and the TM domain (CD8α or CD28) is in italics.

HN3-CD8H-CD8TM
(SEQ ID NO: 1)
CAGGTGCAGCTGGTGCAGTCTGGGGGAGGCTTGGTACAGCCTGGAGGGTC
CCTGAGACTCTCCTGTGCAGCCTCTTATTTCGATTTCGATTCTTATGAAA
TGAGCTGGGTCCGCCAGGCTCCAGGGAAGGGCCTAGAGTGGATTGGGAGT
ATCTATCATAGTGGGAGCACCTACTACAACCCGTCCCTCAAGAGTCGAGT
CACCATCTCCAGAGACAATTCCAAGAACACGCTGTATCTGCAAATGAACA
CCCTGAGAGCCGAGGACACAGCCACGTATTACTGTGCGAGAGTAAATATG
GACCGATTTGACTACTGGGGCCAGGGAACCCTGGTCACCGTCTCCTCAAC
TAGTACCACGACGCCAGCGCCGCGACCACCAACACCGGCGCCCACCATCG
CGTCGCAGCCCCTGTCCCTGCGCCCAGAGGCGTGCCGGCCAGCGGCGGGG
GGCGCAGTGCACACGAGGGGGCTGGACTTCGCCTGTGACATCTACATCTG
GGCGCCCTTGGCCGGGACTTGTGGGGTCCTTCTCCTGTCACTGGTTATCA
CC
HN3-CD8H-CD8TM
(SEQ ID NO: 2)
QVQLVQSGGGLVQPGGSLRLSCAASYFDFDSYEMSWVRQAPGKGLEWIGS
IYHSGSTYYNPSLKSRVTISRDNSKNTLYLQMNTLRAEDTATYYCARVNM
DRFDYWGQGTLVTVSSTSTTTPAPRPPTPAPTIASQPLSLRPEACRPAAG
GAVHTRGLDFACDIYIWAPLAGTCGVLLLSLVIT

| Feature | Residues of SEQ ID NO: 2 |
|---|---|
| HN3 antibody | 1-116 |
| Restriction site | 117-118 |
| CD8 hinge | 119-163 |
| CD8 transmembrane domain | 164-184 |

HN3-CD8H-CD28TM
(SEQ ID NO: 3)
CAGGTGCAGCTGGTGCAGTCTGGGGGAGGCTTGGTACAGCCTGGAGGGTC
CCTGAGACTCTCCTGTGCAGCCTCTTATTTCGATTTCGATTCTTATGAAA
TGAGCTGGGTCCGCCAGGCTCCAGGGAAGGGCCTAGAGTGGATTGGGAGT
ATCTATCATAGTGGGAGCACCTACTACAACCCGTCCCTCAAGAGTCGAGT
CACCATCTCCAGAGACAATTCCAAGAACACGCTGTATCTGCAAATGAACA
CCCTGAGAGCCGAGGACACAGCCACGTATTACTGTGCGAGAGTAAATATG
GACCGATTTGACTACTGGGGCCAGGGAACCCTGGTCACCGTCTCCTCAAC
TAGTACCACTACTCCTGCGCCTAGACCACCCACACCTGCCCCTACGATCG
CTTCACAACCGCTTAGCCTTAGGCCGGAAGCATGTCGGCCAGCGGCGGGG
GGCGCAGTGCACACGAGGGGGCTGGACTTCGCCTGTGACTTCTGGGTGCT
GGTGGTGGTGGGAGGCGTGCTGGCCTGCTATTCCCTGCTGGTGACCGTGG
CCTTCATCATCTTTTGGGTG
HN3-CD8H-CD28TM
(SEQ ID NO: 4)
QVQLVQSGGGLVQPGGSLRLSCAASYFDFDSYEMSWVRQAPGKGLEWIGS
IYHSGSTYYNPSLKSRVTISRDNSKNTLYLQMNTLRAEDTATYYCARVNM
DRFDYWGQGTLVTVSSTSTTTPAPRPPTPAPTIASQPLSLRPEACRPAAG
GAVHTRGLDFACDFWVLVVVGGVLACYSLLVTVAFIIFWV

| Feature | Residues of SEQ ID NO: 4 |
|---|---|
| HN3 antibody | 1-116 |
| Restriction site | 117-118 |
| CD8 hinge | 119-163 |
| CD28 transmembrane domain | 164-190 |

HN3-IgG4H-CD28TM
(SEQ ID NO: 5)
CAGGTGCAGCTGGTGCAGTCTGGGGGAGGCTTGGTACAGCCTGGAGGGTC
CCTGAGACTCTCCTGTGCAGCCTCTTATTTCGATTTCGATTCTTATGAAA
TGAGCTGGGTCCGCCAGGCTCCAGGGAAGGGCCTAGAGTGGATTGGGAGT
ATCTATCATAGTGGGAGCACCTACTACAACCCGTCCCTCAAGAGTCGAGT
CACCATCTCCAGAGACAATTCCAAGAACACGCTGTATCTGCAAATGAACA
CCCTGAGAGCCGAGGACACAGCCACGTATTACTGTGCGAGAGTAAATATG
GACCGATTTGACTACTGGGGCCAGGGAACCCTGGTCACCGTCTCCTCAAC
TAGTGAGAGCAAGTACGGACCACCTTGCCCACCATGTCCATTCTGGGTGC
TGGTGGTGGTGGGAGGCGTGCTGGCCTGCTATTCCCTGCTGGTGACCGTG
GCCTTCATCATCTTTTGGGTG
HN3-IgG4H-CD28TM
(SEQ ID NO: 6)
QVQLVQSGGGLVQPGGSLRLSCAASYFDFDSYEMSWVRQAPGKGLEWIGS
IYHSGSTYYNPSLKSRVTISRDNSKNTLYLQMNTLRAEDTATYYCARVNM
DRFDYWGQGTLVTVSSTSESKYGPPCPPCPFWVLVVVGGVLACYSLLVTV
AFIIFWV

| Feature | Residues of SEQ ID NO: 6 |
|---|---|
| HN3 antibody | 1-116 |
| Restriction site | 117-118 |
| IgG4 hinge | 119-130 |
| CD28 transmembrane domain | 131-157 |

HN3-IgG4H-CD8TM
(SEQ ID NO: 7)
CAGGTGCAGCTGGTGCAGTCTGGGGGAGGCTTGGTACAGCCTGGAGGGTC
CCTGAGACTCTCCTGTGCAGCCTCTTATTTCGATTTCGATTCTTATGAAA
TGAGCTGGGTCCGCCAGGCTCCAGGGAAGGGCCTAGAGTGGATTGGGAGT
ATCTATCATAGTGGGAGCACCTACTACAACCCGTCCCTCAAGAGTCGAGT
CACCATCTCCAGAGACAATTCCAAGAACACGCTGTATCTGCAAATGAACA
CCCTGAGAGCCGAGGACACAGCCACGTATTACTGTGCGAGAGTAAATATG
GACCGATTTGACTACTGGGGCCAGGGAACCCTGGTCACCGTCTCCTCAAC
TAGTGAGAGCAAGTACGGACCACCTTGCCCACCATGTCCAATCTACATCT
GGGCGCCCTTGGCCGGGACTTGTGGGGTCCTTCTCCTGTCACTGGTTATC
ACC
HN3-IgG4H-CD8TM
(SEQ ID NO: 8)
QVQLVQSGGGLVQPGGSLRLSCAASYFDFDSYEMSWVRQAPGKGLEWIGS
IYHSGSTYYNPSLKSRVTISRDNSKNTLYLQMNTLRAEDTATYYCARVNM
DRFDYWGQGTLVTVSSTSESKYGPPCPPCPIYIWAPLAGTCGVLLLSIVI
T

| Feature | Residues of SEQ ID NO: 8 |
|---|---|
| HN3 antibody | 1-116 |
| Restriction site | 117-118 |
| IgG4 hinge | 119-130 |
| CD8 transmembrane domain | 131-151 |

HN3-IgG4H-CH3-CD28TM
(SEQ ID NO: 9)
CAGGTGCAGCTGGTGCAGTCTGGGGGAGGCTTGGTACAGCCTGGAGGGTC
CCTGAGACTCTCCTGTGCAGCCTCTTATTTCGATTTCGATTCTTATGAAA
TGAGCTGGGTCCGCCAGGCTCCAGGGAAGGGCCTAGAGTGGATTGGGAGT
ATCTATCATAGTGGGAGCACCTACTACAACCCGTCCCTCAAGAGTCGAGT
CACCATCTCCAGAGACAATTCCAAGAACACGCTGTATCTGCAAATGAACA
CCCTGAGAGCCGAGGACACAGCCACGTATTACTGTGCGAGAGTAAATATG
GACCGATTTGACTACTGGGGCCAGGGAACCCTGGTCACCGTCTCCTCAAC
TAGtGAGTCCAAGTACGGCCCCCCTTGCCCACCATGTCCAGGACAGCCAA
GGGAGCCTCAGGTGTATACCCTGCCTCCATCTCAGGAGGAGATGACCAAG
AACCAGGTGAGCCTGACATGCCTGGTGAAGGGCTTCTACCCAAGCGACAT
CGCCGTGGAGTGGGAGTCCAATGGCCAGCCCGAGAACAATTACAAGACCA
CACCCCCTGTGCTGGACTCTGATGGCAGCTTCTTTCTGTATTCCCGGCTG
ACCGTGGATAAGTCTAGATGGCAGGAGGGCAACGTGTTCAGCTGCTCCGT
GATGCACGAGGCCCTGCACAATCACTACACACAGAAGTCTCTGAGCCTGT
CCCTGGGCAAGTTTTGGGTGCTGGTGGTGGTGGGAGGCGTGCTGGCCTGT
TATTCTCTGCTGGTGACAGTGGCCTTCATCATCTTTTGGGTG
HN3-IgG4H-CH3-CD28TM
(SEQ ID NO: 10)
QVQLVQSGGGLVQPGGSLRLSCAASYFDFDSYEMSWVRQAPGKGLEWIGS
IYHSGSTYYNPSLKSRVTISRDNSKNTLYLQMNTLRAEDTATYYCARVNM
DRFDYWGQGTLVTVSSTSESKYGPPCPPCPGQPREPQVYTLPPSQEEMTK
NQVSLTCLVKGFYPSDIAVEWESNGQPENNYKTTPPVLDSDGSFFLYSRL
TVDKSRWQEGNVFSCSVMHEALHNHYTQKSLSLSLGKFWVLVVVGGVLAC
YSLLVTVAFIIFWV

| Feature | Residues of SEQ ID NO: 10 |
|---|---|
| HN3 antibody | 1-116 |
| Restriction site | 117-118 |
| IgG4-CH3 hinge | 119-237 |
| CD28 transmembrane domain | 238-264 |

HN3-IgG4H-CH2CH3-CD28TM
(SEQ ID NO: 11)
CAGGTGCAGCTGGTGCAGTCTGGGGGAGGCTTGGTACAGCCTGGAGGGTC
CCTGAGACTCTCCTGTGCAGCCTCTTATTTCGATTTCGATTCTTATGAAA
TGAGCTGGGTCCGCCAGGCTCCAGGGAAGGGCCTAGAGTGGATTGGGAGT
ATCTATCATAGTGGGAGCACCTACTACAACCCGTCCCTCAAGAGTCGAGT
CACCATCTCCAGAGACAATTCCAAGAACACGCTGTATCTGCAAATGAACA
CCCTGAGAGCCGAGGACACAGCCACGTATTACTGTGCGAGAGTAAATATG
GACCGATTTGACTACTGGGGCCAGGGAACCCTGGTCACCGTCTCCTCAAC
TAGTGAGTCCAAGTACGGACCACCTTGCCCACCATGTCCAGCACCTCCAG
TGGCAGGACCAAGCGTGTTCCTGTTTCCACCTAAGCCTAAGGACACCCTG
ATGATCTCTCGGACCCCAGAGGTGACATGCGTGGTGGTGGACGTGAGCCA
GGAGGATCCAGAGGTGCAGTTCAACTGGTACGTGGATGGCGTGGAGGTGC
ACAATGCCAAGACAAAGCCCAGGGAGGAGCAGTTTCAGAGCACCTACCGC
GTGGTGTCCGTGCTGACAGTGCTGCACCAGGACTGGCTGAACGGCAAGGA
GTATAAGTGCAAGGTGTCCAATAAGGGCCTGCCTAGCTCCATCGAGAAGA
CCATCTCTAAGGCAAAGGGACAGCCAAGGGAGCCTCAGGTGTACACACTG
CCACCCAGCCAGGAGGAGATGACCAAGAACCAGGTGTCCCTGACATGTCT
GGTGAAGGGCTTCTATCCCTCCGACATCGCCGTGGAGTGGGAGTCTAATG
GCCAGCCTGAGAACAATTACAAGACCACACCTCCAGTGCTGGACTCCGAT
GGCTCTTTCTTTCTGTATTCTCGGCTGACCGTGGATAAGAGCAGATGGCA
GGAGGGCAACGTGTTCTCTTGCAGCGTGATGCACGAGGCCCTGCACAATC
ACTACACACAGAAGTCCCTGTCTCTGAGCCTGGGCAAGTTTTGGGTGCTG
GTGGTGGTGGGAGGCGTGCTGGCCTGTTATTCCCTGCTGGTGACCGTGGC
CTTCATCATCTTTTGGGTG
HN3-IgG4H-CH2CH3-CD28TM
(SEQ ID NO: 12)
QVQLVQSGGGLVQPGGSLRLSCAASYFDFDSYEMSWVRQAPGKGLEWIGS
IYHSGSTYYNPSLKSRVTISRDNSKNTLYLQMNTLRAEDTATYYCARVNM
DRFDYWGQGTLVTVSSTSESKYGPPCPPCPAPPVAGPSVFLFPPKPKDTL
MISRTPEVTCVVVDVSQEDPEVQFNWYVDGVEVHNAKTKPREEQFQSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGKFWVL
VVVGGVLACYSLLVTVAFIIFWV

| Feature | Residues of SEQ ID NO: 12 |
|---|---|
| HN3 antibody | 1-116 |
| Restriction site | 117-118 |
| IgG4-CH2-CH3 hinge | 119-346 |
| CD28 transmembrane domain | 347-373 |

The CH2 domain included in the HN3-IgG4H-CH2CH3-CD28TM CAR is modified CH2 domain having the following sequence (SBQ ID NO: 59; substituted amino acid residues are underlined):

APPVAGPSVFLFPPKPKDTLMISRTPEVTCVVVDVSQEDPEVQFNWYVDG
VEVHNAKTKPREEQFQSTYRVVSVLTVLHQDWLNGKEYKCKVSNKGLPSS
IEKTISKAK

In some aspects, a wild-type CH2 domain is used in place of the modified CH2 domain, for example a wild-type CH2 domain having the following sequence (SEQ ID NO: 60):

APEFLGGPSVFLFPPKPKDTLMISRTPEVTCVVVDVSQEDPEVQFNWYVD
GVEVHNAKTKPREEQFNSTYRVVSVLTVLHQDWLNGKEYKCKVSNKGLPS
SIEKTISKAK
hYP7-IgG4H-CD28TM
(SEQ ID NO: 13)
GAGGTGCAGCTTGTTGAGTCTGGTGGAGGATTGGTGCAGCCTGGAGGGTC
ATTGAGACTCTCATGTGCAGCCTCTGGATTCACCTTCAATAAGAATGCCA
TGAATTGGGTCCGCCAGGCTCCAGGAAAGGGTTTGGAATGGGTTGGCCGC
ATAAGAAATAAAACTAATAATTATGCAACATATTATGCCGATTCAGTGAA
AGCCAGGTTTACCATCTCCAGAGATGATTCAAAGAACTCACTCTATCTGC
AAATGAACAGCTTGAAAACCGAGGACACAGCCGTGTACTATTGTGTGGCT
GGTAACTCGTTTGCTTACTGGGGCCAAGGGACTCTGGTCACTGTCTCTGC
AGGCGGAGGCGGATCAGGTGGTGGCGGATCTGGAGGTGGCGGAAGCGACA
TTGTGATGACCCAGTCTCCAGACTCCCTAGCTGTGTCACTGGGAGAGAGG
GCCACTATCAACTGCAAGTCCAGTCAGAGCCTTTTATATAGCAGCAATCA
AAAGAACTACTTGGCCTGGTACCAACAGAAACCAGGGCAGCCTCCTAAAC
TGCTGATTTACTGGGCATCCAGTAGGGAATCTGGGGTCCCTGATCGCTTC
AGTGGCAGTGGATCTGGGACAGATTTCACTCTCACCATCAGCAGTCTGCA
GGCTGAAGACGTGGCAGTTTATTACTGTCAGCAATATTATAACTATCCGC
TCACGTTCGGTCAGGGGACCAAGTTGGAGATCAAAACTAGTGAGAGCAAG
TACGGACCACCTTGCCCACCATGTCCATTCTGGGTGCTGGTGGTGGTGGG
AGGCGTGCTGGCCTGCTATTCCCTGCTGGTGACCGTGGCCTTCATCATCT
TTTGGGTG
hYP7-IgG4H-CD28TM
(SEQ ID NO: 14)
EVQLVESGGGLVQPGGSLRLSCAASGFTFNKNAMNWVRQAPGKGLEWVGR
IRNKTNNYATYYADSVKARFTISRDDSKNSLYLQMNSLKTEDTAVYYCVA
GNSFAYWGQGTLVTVSAGGGGSGGGGSGGGGSDIVMTQSPDSLAVSLGER
ATINCKSSQSLLYSSNQKNYLAWYQQKPGQPPKLLIYWASSRESGVPDRE
SGSGSGTDFTLTISSLQAEDVAVYYCOQYYNYPLTFGQGTKLEIKTSESK
YGPPCPPCPFWVLVVVGGVLACYSLLVTVAFIIFWV

| Feature | Residues of SEQ ID NO: 14 |
|---|---|
| hYP7 (VH-linker-VL) | 1-245 |
| Restriction site | 246-247 |
| IgG4 hinge | 248-259 |
| CD28 transmembrane domain | 260-286 |

hYP7-CD8H-CD8TM
(SEQ ID NO: 15)
GAGGTGCAGCTTGTTGAGTCTGGTGGAGGATTGGTGCAGCCTGGAGGGTC
ATTGAGACTCTCATGTGCAGCCTCTGGATTCACCTTCAATAAGAATGCCA
TGAATTGGGTCCGCCAGGCTCCAGGAAAGGGTTTGGAATGGGTTGGCCGC
ATAAGAAATAAAACTAATAATTATGCAACATATTATGCCGATTCAGTGAA
AGCCAGGTTTACCATCTCCAGAGATGATTCAAAGAACTCACTCTATCTGC
AAATGAACAGCTTGAAAACCGAGGACACAGCCGTGTACTATTGTGTGGCT
GGTAACTCGTTTGCTTACTGGGGCCAAGGGACTCTGGTCACTGTCTCTGC
AGGCGGAGGCGGATCAGGTGGTGGCGGATCTGGAGGTGGCGGAAGCGACA
TTGTGATGACCCAGTCTCCAGACTCCCTAGCTGTGTCACTGGGAGAGAGG
GCCACTATCAACTGCAAGTCCAGTCAGAGCCTTTTATATAGCAGCAATCA
AAAGAACTACTTGGCCTGGTACCAACAGAAACCAGGGCAGCCTCCTAAAC
TGCTGATTTACTGGGCATCCAGTAGGGAATCTGGGGTCCCTGATCGCTTC
AGTGGCAGTGGATCTGGGACAGATTTCACTCTCACCATCAGCAGTCTGCA
GGCTGAAGACGTGGCAGTTTATTACTGTCAGCAATATTATAACTATCCGC
TCACGTTCGGTCAGGGGACCAAGTTGGAGATCAAAACTAGTACCACGACG
CCAGCGCCGCGACCACCAACACCGGCGCCCACCATCGCGTCGCAGCCCCT
GTCCCTGCGCCCAGAGGCGTGCCGGCCAGCGGCGGGGGGCGCAGTGCACA
CGAGGGGGCTGGACTTCGCCTGTGACATCTACATCTGGGCGCCCTTGGCC
GGGACTTGTGGGGTCCTTCTCCTGTCACTGGTTATCACC
hYP7-CD8H-CD8TM
(SEQ ID NO: 16)
EVQLVESGGGLVQPGGSLRLSCAASGFTFNKNAMNWVRQAPGKGLEWVGR
IRNKTNNYATYYADSVKARFTISRDDSKNSLYLQMNSLKTEDTAVYYCVA
GNSFAYWGQGTLVTVSAGGGGSGGGGSGGGGSDIVMTQSPDSLAVSLGER
ATINCKSSQSLLYSSNQKNYLAWYQQKPGQPPKLLIYWASSRESGVPDRF
SGSGSGTDFTLTISSLQAEDVAVYYCQQYYNYPLTFGQGTKLEIKTSTTT
PAPRPPTPAPTIASQPLSLRPEACRPAAGGAVHTRGLDFACDIYIWAPLA
GTCGVLLLSLVIT

| Feature | Residues of SEQ ID NO: 16 |
|---|---|
| hYP7 (VH-linker-VL) | 1-245 |
| Restriction site | 246-247 |
| CD8 hinge | 248-292 |
| CD8 transmembrane domain | 293-313 |

### VI. Chimeric Antigen Receptors (CARs)

Disclosed herein are GPC3-specific CARs and cells (for example, T cells, B cells, NK cells, macrophages and iPSCs) engineered to express CARs. Generally, CARs include a binding moiety, an extracellular hinge/spacer element, a transmembrane region and an intracellular domain that performs signaling functions (Cartellieri et al., J Biomed Biotechnol 2010:956304, 2010; Dai et al., J Natl Cancer Inst 108(7):djv439, 2016). In many instances, the binding moiety is an antigen binding fragment of a monoclonal antibody, such as a scFv or single-domain antibody. The spacer/hinge region typically includes sequences from IgG subclasses, such as IgG1, IgG4, IgD and CD8 domains. In some aspects herein, the hinge region includes (or consists of) a modified IgG4 hinge sequence set forth as SEQ ID NO: 43. In other aspects herein, the hinge region includes (or consists of) a wild-type IgG4 hinge sequence set forth as SEQ ID NO: 52. The transmembrane domain can be can derived from a variety of different T cell proteins, such as CD3ζ, CD4, CD8, CD28 or inducible T cell co-stimulator (ICOS). Several different endodomains have been used to generate CARs. For example, the endodomain can consist of a signaling chain having an ITAM, such as CD3ζ or FcεRIγ. In some instances, the endodomain further includes the intracellular portion of at least one additional co-stimulatory domain, such as CD28, 4-1BB (CD137, TNFRSF9), OX-40 (CD134), ICOS, CD27, MYD88-CD40, killer cell immunoglobulin-like receptor 2DS2 (KIR2DS2) and/or DAP10.

The CAR can also include a signal peptide sequence, e.g., N-terminal to the antigen binding domain. The signal peptide sequence can be any suitable signal peptide sequence, such as a signal sequence from granulocyte-macrophage colony-stimulating factor receptor (GMCSFR), immunoglobulin light chain kappa, or IL-2. While the signal peptide sequence may facilitate expression of the CAR on the surface of the cell, the presence of the signal peptide sequence in an expressed CAR is not necessary in order for the CAR to function. Upon expression of the CAR on the cell surface, the signal peptide sequence may be cleaved off of the CAR. Accordingly, in some aspects, the CAR lacks a signal peptide sequence.

In some aspects, the CARs disclosed herein are expressed from a construct (such as from a lentivirus vector) that also expresses a truncated version of human EGFR (huEGFRt; discussed in more detail in section VII below). The CAR and huEGFRt are separated by a self-cleaving peptide sequence (such as T2A) such that upon expression in a transduced cell, the CAR is cleaved from huEGFRt (see WO 2019/094482).

In some aspects disclosed herein, the CAR constructs encode the following amino acid sequences, in the N-terminal to C-terminal direction:
GMCSFRss: MLLLVTSLLLCELPHPAFLLIP (SEQ ID NO: 44)
NdeI: HM
Antigen-binding: scFv or single-domain antibody sequence
SpeI: TS
Hinge: modified IgG4 (ESKYGPPCPPCP; SEQ ID NO: 43), wild-type IgG4 (ESKYGPPCPSCP; SEQ ID NO: 52) or CD8α (TTTPAPRPPTPAPTIASQPLSLRPEACRPAAGGAVHTRGLDFACD; SEQ ID NO: 45)
TM: CD28 (FWVLVVVGGVLACYSLLVTVAFIIFWV; SEQ ID NO: 46) or CD8α (IYIWAPLAGTCGVLLLSLVIT; SEQ ID NO: 47)
4-1BB: KRGRKKLLYIFKQPFMRPVQTTQEEDGCSCRFPEEEEGGCEL (SEQ ID NO: 48)
CD3ζ: RVKFSRSADAPAYQQGQNQLYNELNLGRREEYDVLDKRRGRDPEMGGKPRRKNPQEGLYNELQK DKMAEAYSEIGMKGERRRGKGHDGLYQGLSTATKDTYDALHMQALPPR (SEQ ID NO: 49)
T2A: EGRGSLLTCGDVEENPGP (SEQ ID NO: 50)
GMCSFRss: MLLLVTSLLLCELPHPAFLLIP (SEQ ID NO: 44)
huEGFRt: RKVCNGIGIGEFKDSLSINATNIKHFKNCTSISGDLHILPVAFRGDSFTHTPPLDPQELDILKTVKEITG FLLIQAWPENRTDLHAFENLEIIRGRTKQHGQFSLAVVSLNITSLGLRSLKEISDGDVIISGNKNLCYA NTINWKKLFGTSGQKTKIISNRGENSCKATGQVCHALCSPEGCWGPEPRDCVSCRNVSRGRECVDK CNLLEGEPREFVENSECIQCHPECLPQAMNITCTGRGPDNCIQCAHYIDGPHCVKTCPAGVMGENN TLVWKYADAGHVCHLCHPNCTYGCTGPGLEGCPTNGPKIPSIATGMVGALLLLLVVALGIGLFM (SEQ ID NO: 51)

Immune cells (such as T cells, B cells, NK cells or macrophages) or iPSCs expressing the CARs disclosed herein can be used to target a specific cell type, such as a tumor cell, for example a GPC3-positive tumor cell. The use of immune cells (such as T cells) expressing CARs is more universal than standard CTL-based immunotherapy because immune cells expressing CARs are HLA unrestricted and can therefore be used for any patient having a tumor that expresses the target antigen.

Accordingly, provided herein are CARs that include a GPC3-specific antibody (or binding fragment thereof). Also provided are isolated nucleic acid molecules and vectors encoding the CARs, and host cells, such as T cells, B cells, NK cells, macrophages or iPSCs, expressing the CARs. Cells expressing CARs comprised of a GPC3-specific monoclonal antibody can be used for the treatment of cancers that express GPC3, such as HCC, melanoma, ovarian clear-cell carcinoma, yolk sac tumor (YST), neuroblastoma, hepatoblastoma, Wilms' tumor, squamous cell carcinoma of the lung, testicular nonseminomatous germ cell tumor, liposarcoma, cervical intraepithelial neoplasia, adenoma of the adrenal gland, schwannoma or embryonal tumor.

### VII. Truncated Human EGFR (huEGFRt)

The human epidermal growth factor receptor is comprised of four extracellular domains, a transmembrane domain and three intracellular domains. The EGFR domains are found in the following N-terminal to C-terminal order: Domain I-Domain II-Domain III-Domain IV-transmembrane (TM) domain-juxtamembrane domain-tyrosine kinase domain-C-terminal tail. Domain I and Domain III are leucine-rich domains that participate in ligand binding. Domain II and Domain IV are cysteine-rich domains and do not make contact with EGFR ligands. Domain II mediates formation of homo-or hetero-dimers with analogous domains from other EGFR family members, and Domain IV can form disulfide bonds with Domain II. The EGFR TM domain makes a single pass through the cell membrane and may play a role in protein dimerization. The intracellular domain includes the juxtamembrane domain, tyrosine kinase domain and C-terminal tail, which mediate EGFR signal transduction (Wee and Wang, Cancers 9(52), doi: 10.3390/cancers9050052; Ferguson, Annu Rev Biophys 37:353-373, 2008; Wang et al., Blood 118(5):1255-1263, 2011).

A truncated version of human EGFR, referred to herein as “huEGFRt” includes only Domain III, Domain IV and the TM domain. Thus, huEGFRt lacks Domain I, Domain II, and all three intracellular domains. huEGFRt is not capable of binding EGF and lacks signaling activity. However, this molecule retains the capacity to bind particular EGFR-specific monoclonal antibodies, such as FDA-approved cetuximab (PCT Publication No. WO 2011/056894).

Transduction of immune cells (such as T cells, B cells, NK cells or macrophages) or iPSCs with a construct (such as a lentivirus vector) encoding both huEGFRt and a GPC3-specific CAR disclosed herein allows for selection of transduced cells using labelled EGFR monoclonal antibody cetuximab (ERBITUX™). For example, cetuximab can be labeled with biotin and transduced cells can be selected using anti-biotin magnetic beads, which are commercially available (such as from Miltenyi Biotec). Co-expression of huEGFRt also allows for in vivo tracking of adoptively transferred CAR-expressing cells. Furthermore, binding of cetuximab to cells expressing huEGFRt induces cytotoxicity of ADCC effector cells, thereby providing a mechanism to eliminate transduced immune cells or iPSCs in vivo (Wang et al., Blood 118(5):1255-1263, 2011), such as at the conclusion of therapy.

In some aspects herein, the amino acid sequence of huEGFRt is at least 80%, at least 85%, at least 90%, at least 95%, at least 96%, at least 97%, at least 98% or at least 99% identical to SEQ ID NO: 51. In some examples, the amino acid sequence of huEGFRt comprises or consists of SEQ ID NO: 51. In other aspects, the amino acid sequence of huEGFRt comprises no more than 10, no more than 9, no more than 8, no more than 7, no more than 6, no more than 5, no more than 4, no more than 3, no more than 2 or no more than 1 amino acid substitutions relative to SEQ ID NO: 51. In some examples, the amino acid substitutions are conservative substitutions.

### VIII. CAR-Expressing Cell Compositions

Compositions are provided that include CAR-expressing cells in combination with one or more pharmaceutically or physiologically acceptable carriers, diluents or excipients. The CAR-expressing cells can be iPSCs, T cells, such as CD3⁺ T cells, such as CD4⁺ and/or CD8⁺ T cells, B cells, NK cells, macrophages or any other suitable immune cell. Such compositions may include buffers such as neutral buffered saline, phosphate buffered saline and the like; carbohydrates such as glucose, mannose, sucrose, dextrans, or mannitol; proteins; polypeptides or amino acids such as glycine; antioxidants; chelating agents such as EDTA or glutathione; adjuvants (e.g., aluminum hydroxide); and preservatives. In some examples the cell-containing composition includes a cryopreservative, such as DMSO or glycerol. In some examples the cell-containing composition includes a culture media, such as DMEM or RPMI, and may further include serum, such as FBS. In some examples the cell-containing composition is frozen or in a liquid form. The cells can be autologous to the recipient. However, the cells can also be heterologous (allogeneic).

With regard to the cells, a variety of aqueous carriers can be used, for example, buffered saline and the like, for introducing the cells. These solutions are sterile and generally free of undesirable matter. These compositions may be sterilized by conventional sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration in these formulations can vary widely, and will be selected primarily based on fluid volumes, viscosities, body weight and the like in accordance with the particular mode of administration selected and the subject's needs.

The precise amount of the composition to be administered can be determined by a physician with consideration of individual differences in age, weight, tumor size/burden, extent of metastasis, and condition of the patient (subject). It can generally be stated that a pharmaceutical composition that includes the CAR-expressing immune cells (T cells, B cells, macrophages and/or NK cells) or iPSCs described herein may be administered at a dosage of 10⁴ to 10⁹ cells/kg body weight, such as 10⁵ to 10⁶ cells/kg body weight, including all integer values within those ranges. Exemplary doses are 10⁶ cells/kg to about 10⁸ cells/kg, such as from about 5×10⁶ cells/kg to about 7.5×10⁷ cells/kg, such as at about 2.5×10⁷ cells/kg, or at about 5.0×10⁷ cells/kg.

A composition can be administered once or multiple times, such as 2, 3, 4, 5, 6, 7, 8, 9 or 10 times at these dosages. The composition can be administered using known immunotherapy infusion techniques (see, e.g., Rosenberg et al., New Eng. J. of Med. 319:1676, 1988). The compositions can be administered daily, weekly, bimonthly or monthly. In some non-limiting examples, the composition is formulated for intravenous administration and is administered multiple times. The quantity and frequency of administration will be determined by such factors as the condition of the subject, and the type and severity of the subject's disease, although appropriate dosages may be determined by clinical trials.

In some aspects, the CAR-encoding nucleic acid molecule is introduced into cells, such as T cells, B cells, NK cells, macrophages or iPSCs, and the subject receives an initial administration of cells, and one or more subsequent administrations of the cells, wherein the one or more subsequent administrations are administered less than 15 days, e.g., 14, 13, 12, 11, 10, 9, 8, 7, 6, 5, 4, 3, or 2 days after the previous administration. In one aspect, more than one administration of the CAR-expressing cells are administered to the subject per week, e.g., 2, 3, or 4 administrations of the CAR-expressing cells of the disclosure are administered per week. In one aspect, the subject receives more than one administration of the CAR-expressing cells per week (e.g., 2, 3 or 4 administrations per week) (also referred to as a cycle), followed by a week of no CAR-expressing cell administrations, and then one or more additional administration of the CAR-expressing cells (e.g., more than one administration of the CAR-expressing cells per week) is administered to the subject. In another aspect, the subject (e.g., a human subject) receives more than one cycle of CAR-expressing cells, and the time between each cycle is less than 10, 9, 8, 7, 6, 5, 4, or 3 days. In one aspect, the CAR-expressing cells are administered every other day for 3 administrations per week. In another aspect, the CAR-expressing cells are administered for at least two, three, four, five, six, seven, eight or more weeks. The dosage of the above treatments to be administered to a patient will vary with the precise nature of the condition being treated and the recipient of the treatment. The scaling of dosages for human administration can be performed according to accepted practices.

In some aspects, CAR-expressing cells are able to replicate in vivo resulting in long-term persistence that can lead to sustained tumor control. In various aspects, the iPSCs, T cells, B cells, macrophages or NK cells administered to the subject, or the progeny of these cells, persist in the subject for at least four months, five months, six months, seven months, eight months, nine months, ten months, eleven months, twelve months, thirteen months, fourteen months, fifteen months, sixteen months, seventeen months, eighteen months, nineteen months, twenty months, twenty-one months, twenty-two months, twenty-three months, or for years after administration of the cells to the subject. In other aspects, the cells and their progeny are present for less than six months, five months, four months, three months, two months, or one month, e.g., three weeks, two weeks, one week, after administration of the CAR-expressing cells to the subject.

The administration of the compositions may be carried out in any convenient manner, including by injection, ingestion, transfusion, implantation or transplantation. The disclosed compositions can be administered to a patient trans-arterially, subcutaneously, intradermally, intratumorally, intranodally, intramedullary, intramuscularly, intra-arterially (including into the hepatic artery (such as HAI) or the femoral artery), by intravenous (i.v.) injection, intraprostatically (e.g., for a prostate cancer), or intraperitoneally. In some aspects, the compositions are administered to a patient by intradermal or subcutaneous injection. In other aspects, the compositions of the disclosure are administered by i.v. injection. In other aspects, the compositions of the disclosure are administered by intra-arterial injection. The compositions can also be injected directly into a tumor or lymph node.

In some aspects, subjects can undergo leukapheresis, wherein leukocytes are collected, enriched, or depleted ex vivo to select and/or isolate the cells of interest, e.g., iPSCs, T cells, B cells, macrophages and/or NK cells. These cell isolates may be expanded by known methods and treated such that one or more CAR constructs can be introduced, thereby creating an autologous cell that expresses the CAR. In some aspects herein, CAR-expressing cells are generated using lentiviral vectors expressing the CAR and a truncated form of the human EGFR (huEGFRt). Co-expression of huEGFRt allows for selection and purification of CAR-expressing immune cells using an antibody that recognizes huEGFRt (e.g., cetuximab, see PCT Publication No. WO 2011/056894), which is described above in section VIII.

In some aspects, immune cells (such as T cells, B cells, NK cells and/or macrophages) are isolated from peripheral blood by lysing the red blood cells and in some instances depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45RO+ T cells, can be further isolated by positive or negative selection techniques. For example, T cells can isolated by incubation with anti-CD3/anti-CD28 (e.g., 3×28)-conjugated beads, such as DYNABEADS® M-450 CD3/CD28 T, for a time period sufficient for positive selection of the desired T cells, see U.S. Published Application No. US20140271635 A1. In a non-limiting example, the time period is about 30 minutes. In other non-limiting examples, the time period ranges from 30 minutes to 36 hours or longer and all integer values there between. In further non-limiting examples, the time period is at least 1, 2, 3, 4, 5, or 6 hours, 10 to 24 hours, 24 hours or longer. Longer incubation times can be used to isolate T cells in any situation where there are few T cells as compared to other cell types, such in isolation from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells, subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. Multiple rounds of selection can also be used.

Enrichment of a cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ T cells by negative selection, a monoclonal antibody cocktail typically includes antibodies to CD14, CD20, CD11b, CD16, HLA-DR, and CD8. A T cell population can be selected that expresses one or more cytokines. Methods for screening for cell expression are disclosed in PCT Publication No. WO 2013/126712.

For isolation of a desired population of cells by positive or negative selection, the concentration of cells and surface (e.g., particles such as beads) can be varied to ensure maximum contact of cells and beads. In some aspects, a concentration of 1 billion cells/ml is used. In further aspects, greater than 100 million cells/ml is used. In other aspects, a concentration of cells of 10, 15, 20, 25, 30, 35, 40, 45, 50, 55, 60, 65, 70, 75, 80, 85, 90, 95, or 100 million cells/ml is used. Without being bound by theory, using high concentrations can result in increased cell yield, cell activation, and cell expansion. Lower concentrations of cells can also be used. Without being bound by theory, significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In some aspects, the concentration of cells used is 5×10⁶/ml. In other aspects, the concentration used can be from about 1×10⁵/ml to 1×10⁶/ml, and any integer value in between.

### IX. Methods of Treatment

Provided herein are methods of treating cancer in a subject by administering to the subject a therapeutically effective amount of a GPC3-targeted CAR-expressing immune cell (such as a T cell, B cell, NK cell or macrophage) of CAR-expressing iPSC disclosed herein. Also provided herein is a method of inhibiting tumor growth or metastasis in a subject by administering to the subject a therapeutically effective amount of a GPC3-targeted CAR-expressing cell disclosed herein. Thus, in some examples, the methods decrease the size, volume and/or weight of a tumor by at least 10%, at least 20%, at least 30%, at least 50%, at least 50%, at least 75%, at least 90%, at least 95%, at least 98%, at least 99% or 100%, for example relative to the size, volume and/or weight of the tumor prior to treatment. In some examples, the methods decrease the size, volume and/or weight of a metastasis by at least 10%, at least 20%, at least 30%, at least 50%, at least 50%, at least 75%, at least 90%, at least 95%, at least 98%, at least 99% or 100%, for example relative to the size, volume and/or weight of the metastasis prior to treatment. In some examples, the methods increase the survival time of a subject with a GPC3-positive cancer by at least 3 months, at least 6 months, at least 9 months, at least 12 months, at least 18 months, at least 24 months, at last 36 months, at least 48 months, or at least 60 months, for example relative to the survival time in an absence of the treatment provided herein. In some examples, combinations of these effects are achieved.

Specifically provided is a method of treating a GPC3-positive cancer in a subject. In some aspects, the method includes administering to the subject a therapeutically effective amount of an isolated immune cell or iPSC that includes a nucleic acid molecule encoding a GPC3-targeted CAR and a huEGFRt, or administering a therapeutically effective amount of an isolated immune cell or iPSC co-expressing a GPC3-targeted CAR and a huEGFRt. In some aspects, the GPC3-positive cancer is hepatocellular carcinoma (HCC), melanoma, ovarian clear-cell carcinoma, yolk sac tumor (YST), neuroblastoma, hepatoblastoma, Wilms' tumor, squamous cell carcinoma of the lung, testicular nonseminomatous germ cell tumor, liposarcoma, cervical intraepithelial neoplasia, adenoma of the adrenal gland, schwannoma or embryonal tumor. In a specific aspect, the cancer treated is HCC.

In some aspects of the methods disclosed herein, the isolated immune cells are T lymphocytes. In some examples, the T lymphocytes are autologous T lymphocytes. In other aspects, the isolated host cells are B cells, NK cells or macrophages.

A therapeutically effective amount of a CAR-expressing immune cell or iPSC will depend upon the severity of the disease, the type of disease, and the general state of the patient's health. A therapeutically effective amount of CAR-expressing cells and compositions thereof is that which provides either subjective relief of a symptom(s) or an objectively identifiable improvement as noted by the clinician or other qualified observer (such as a decrease in tumor volume or metastasis).

Administration of the CAR-expressing cells and compositions disclosed herein can also be accompanied by administration of other anti-cancer agents or therapeutic treatments (such as surgical resection of a tumor). Any suitable anti-cancer agent can be administered in combination with the compositions disclosed herein. Exemplary anti-cancer agents include, but are not limited to, chemotherapeutic agents, such as, for example, mitotic inhibitors, alkylating agents, anti-metabolites, intercalating antibiotics, growth factor inhibitors, cell cycle inhibitors, enzymes, topoisomerase inhibitors, anti-survival agents, biological response modifiers, anti-hormones (e.g., anti-androgens) and anti-angiogenesis agents. Other anti-cancer treatments include radiation therapy and antibodies (e.g., mAbs) that specifically target cancer cells or other cells (e.g., anti-PD-1, anti-CLTA4, anti-EGFR, or anti-VEGF). In one example, a cancer is treated by administering a GPC3-targeted CAR immune cell (such as iPSC, T cell, B cell, NK cell or macrophage) disclosed herein and one or more therapeutic mAbs, such as one or more of a PD-L1 antibody (e.g., durvalumab, KN035, cosibelimab, BMS-936559, BMS935559, MEDI-4736, MPDL-3280A, or MEDI-4737), or CLTA-4 antibody (e.g., ipilimumab or tremelimumab). In one example, a cancer is treated by administering a GPC3-targeted CAR immune cell (such as iPSC, T cell, B cell, NK cell or macrophage) disclosed herein and one or more mAbs, for example: 3F8, Abagovomab, Adecatumumab, Afutuzumab, Alacizumab, Alemtuzumab, Altumomab pentetate, Anatumomab mafenatox, Apolizumab, Arcitumomab, Bavituximab, Bectumomab, Belimumab, Besilesomab, Bevacizumab, Bivatuzumab mertansine, Blinatumomab, Brentuximab vedotin, Cantuzumab mertansine, Capromab pendetide, Catumaxomab, CC49, Cetuximab, Citatuzumab bogatox, Cixutumumab, Clivatuzumab tetraxetan, Conatumumab, Dacetuzumab, Detumomab, Ecromeximab, Eculizumab, Edrecolomab, Epratuzumab, Ertumaxomab, Etaracizumab, Farletuzumab, Figitumumab, Galiximab, Gemtuzumab ozogamicin, Girentuximab, Glembatumumab vedotin, Ibritumomab tiuxetan, Igovomab, Imciromab, Intetumumab, Inotuzumab ozogamicin, Ipilimumab, Iratumumab, Labetuzumab, Lexatumumab, Lintuzumab, Lorvotuzumab mertansine, Lucatumumab, Lumiliximab, Mapatumumab, Matuzumab, Mepolizumab, Metelimumab, Milatuzumab, Mitumomab, Morolimumab, Nacolomab tafenatox, Naptumomab estafenatox, Necitumumab, Nimotuzumab, Nofetumomab merpentan, Ofatumumab, Olaratumab, Oportuzumab monatox, Oregovomab, Panitumumab, Pemtumomab, Pertuzumab, Pintumomab, Pritumumab, Ramucirumab, Rilotumumab, Rituximab, Robatumumab, Satumomab pendetide, Sibrotuzumab, Sonepcizumab, Tacatuzumab tetraxetan, Taplitumomab paptox, Tenatumomab, TGN1412, Ticilimumab (tremelimumab), Tigatuzumab, TNX-650, Trastuzumab, Tremelimumab, Tucotuzumab celmoleukin, Veltuzumab, Volociximab, Votumumab, Zalutumumab, or combinations thereof.

In one example, a cancer is treated by administering a GPC3-targeted CAR immune cell (such as iPSC, T cell, B cell, NK cell or macrophage) disclosed herein and one or more alkylating agents, such as nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine).

In one example, a cancer is treated by administering a GPC3-targeted CAR immune cell (such as iPSC, T cell, B cell, NK cell or macrophage) disclosed herein and one or more antimetabolites, such as folic acid analogs (such as methotrexate), pyrimidine analogs (such as 5-FU or cytarabine), and purine analogs, such as mercaptopurine or thioguanine.

In one example, a cancer is treated by administering a GPC3-targeted CAR immune cell (such as iPSC, T cell, B cell, NK cell or macrophage) disclosed herein and one or more natural products, such as include vinca alkaloids (such as vinblastine, vincristine, or vindesine), epipodophyllotoxins (such as etoposide or teniposide), antibiotics (such as dactinomycin, daunorubicin, doxorubicin, bleomycin, plicamycin, or mitomycin C), and enzymes (such as L-asparaginase).

In one example, a cancer is treated by administering a GPC3-targeted CAR immune cell (such as iPSC, T cell, B cell, NK cell or macrophage) disclosed herein and one or more platinum coordination complexes (such as cis-diamine-dichloroplatinum II also known as cisplatin), substituted ureas (such as hydroxyurea), methyl hydrazine derivatives (such as procarbazine), and adrenocrotical suppressants (such as mitotane and aminoglutethimide).

In one example, a cancer is treated by administering a GPC3-targeted CAR immune cell (such as iPSC, T cell, B cell, NK cell or macrophage) disclosed herein and one or more hormones or antagonists, such as adrenocorticosteroids (such as prednisone), progestins (such as hydroxyprogesterone caproate, medroxyprogesterone acetate, and magestrol acetate), estrogens (such as diethylstilbestrol and ethinyl estradiol), antiestrogens (such as tamoxifen), and androgens (such as testerone proprionate and fluoxymesterone).

In one example, a cancer is treated by administering a GPC3-targeted CAR immune cell (such as iPSC, T cell, B cell, NK cell or macrophage) disclosed herein and one or more chemotherapy drugs, such as Adriamycin, Alkeran, Ara-C, BiCNU, Busulfan, CCNU, Carboplatinum, Cisplatinum, Cytoxan, Daunorubicin, DTIC, 5-FU, Fludarabine, Hydrea, Idarubicin, Ifosfamide, Methotrexate, Mithramycin, Mitomycin, Mitoxantrone, Nitrogen Mustard, Taxol (or other taxanes, such as docetaxel), Velban, Vincristine, VP-16, Gemcitabine (Gemzar), Herceptin, Irinotecan (Camptosar, CPT-11), Leustatin, Navelbine, Rituxan STI-571, Taxotere, Topotecan (Hycamtin), Xeloda (Capecitabine), Zevelin and calcitriol.

In one example, a cancer is treated by administering a GPC3-targeted CAR immune cell (such as iPSC, T cell, B cell, NK cell or macrophage) disclosed herein and one or more immunomodulators, such as AS-101 (Wyeth-Ayerst Labs.), bropirimine (Upjohn), gamma interferon (Genentech), GM-CSF (granulocyte macrophage colony stimulating factor; Genetics Institute), IL-2 (Cetus or Hoffman-LaRoche), human immune globulin (Cutter Biological), IMREG (from Imreg of New Orleans, La.), SK&F 106528, and TNF (tumor necrosis factor; Genentech).

Another treatment that can be used in combination with those provided herein is surgical treatment, for example surgical resection of the cancer or a portion of it. Another example of a treatment is radiotherapy, for example administration of radioactive material or energy (such as external beam therapy) to the tumor site to help eradicate the tumor or shrink it prior to surgical resection.

In a specific example, the method includes treating an HCC by administering to the subject a therapeutically effective amount of (1) an isolated immune cell or iPSC that includes a nucleic acid molecule encoding a GPC3-targeted CAR and a huEGFRt, or administering a therapeutically effective amount of an isolated immune cell or iPSC co-expressing a GPC3-targeted CAR and a huEGFRt. In some examples, the method further includes administering to the subject a therapeutically effective amount of one or more other chemotherapeutic or biological agents. In some aspects, the one or more other chemotherapeutic or biological agents is one or more of 5-FU, cisplatin, gemcitabine, oxaliplatin, doxorubicin, capecitabine, floxuridine, or mitoxantrone, such as gemcitabine plus oxaliplatin (GEMOS), floxuridine, cisplatin, and oxaliplatin, or 5-FU, oxaliplatin and leucovorin (FOLFOX). In some aspects, the one or more other chemotherapeutic or biological agents is one or more of sorafenib, lenvatinib, regorafenib, cabozantinib and ramucirumab. In some aspects, the one or more other chemotherapeutic or biological agents is an immunotherapy drug, such as pembrolizumab and/or nivolumab.

The following examples are provided to illustrate certain particular features and/or aspects. These examples should not be construed to limit the disclosure to the particular features or aspects described.

### EXAMPLES

The fully human HN3 nanobody has several attractive features, including the ability to mitigate immunogenicity concerns in humans; cross species binding to mouse and human GPC3; and Wnt blocking capability (Li et al., Hepatology 70(4):1231-1245, 2019; Fleming et al., Hepatology 71(5):1696-1711, 2020; Feng et al., Proc Natl Acad Sci USA 110(12):E1083-E1091, 2013; Chen et al., Cancer Immunol Immunother 66(4):475-489, 2017; Phung et al., mAbs 4(5):592-599, 2012; Gao et al., Hepatology 60(1527-3350):576-587, 2014; Zhang and Ho, Sci Rep 6:633878, 2016). The fully human nature of HN3 limits immunogenicity constraints in comparison to mouse-derived scFvs. The cross-species feature allows swift preclinical testing in mouse models and human studies without changing nanobody content. Finally, the HN3 nanobody specifically targets, and inactivates the native Wnt binding domain of GPC3. Leveraging the natural binding region is beneficial because upregulation of Wnt signaling is a significant factor in HCC pathogenesis (de La Coste et al., Proc Natl Acad Sci USA 95(15):8847-8851, 1998; Cancer Genome Atlas Research Network, Cell 169(7):1327-1341, 2017; Laurent-Puig et al., Gastroenterology 120(7):1763-1773, 2001; Zucman-Rossi et al., Hepatology 43(3):515-524, 2006; Capurro et al., Cancer Res 65(14):6245-6254, 2005).

The following examples describe the rational design GPC3-targeted CARs using the HN3 nanobody or hYP7 scFv as the targeting element to traffic to GPC3-positive tumor cells. It is demonstrated herein that maximal antitumor efficacy is achieved by precise engineering of the hinge and transmembrane domains. By dissecting the contribution of each hinge and transmembrane component, it was determined that the hinge and transmembrane work synergistically.

The data described below demonstrates that HN3 engineered T cells containing an IgG4 hinge and a CD28 transmembrane induced swift, potent, and durable antitumor responses. Further, these antitumor responses were correlated with a specific CD8+ Tₑₘᵣₐ signature not observed with other CAR T cells.

### Example 1: Materials and Methods

This example describes the materials and experimental procedures used in the studies described in Examples 2-6.

### Cell Models

The Hep3B (HCC), HepG2 (hepatoblastoma), HEK-293T, and Jurkat cell lines were purchased from American Type Culture Collection (Manassas, VA). Hep3B and HepG2 were transduced with lentiviruses expressing luciferase. The Hep3BKO GFP-luciferase cell line was established using CRISPR/CAS9 knockout as previously described. The cell lines were cultured in Dulbecco's modified Eagle medium supplemented with 10% fetal bovine serum (FBS), 1% L-glutamine, and 1% penicillin-streptomycin at 37° C. in a humidified atmosphere with 5% CO₂. Peripheral blood mononuclear cells (PBMCs) from peripheral blood of healthy donors were isolated using Ficoll (GE Healthcare, Chicago, IL) according to the manufacturer's instructions. Jurkat cells were grown in RPMI-1640 medium supplemented with 10% fetal bovine serum, 1% L-glutamine, and 1% penicillin-streptomycin at 37° C. in a humidified atmosphere with 5% CO₂.

### Jurkat Binding and Jurkat NFκB Reporter

Jurkat cells were transduced with CAR-containing lentiviruses at a multiplicity of infection (MOI) of 5. After 7 days, cell-surface EGFRt was assessed using anti-human IgG-phycoerythrin (APC)-conjugated antibody. Cell surface binding of GPC3 protein tagged with hFc (ACRO) to CAR-expressing Jurkat cells was assessed using anti-human IgG-phycoerythrin (APC)-conjugated antibody (Jackson ImmunoResearch, West Grove, PA).

### CAR T cell Production

CAR T cells were produced as described previously (Li et al., Gastroenterology 158(8):2250-2265, 2020). Briefly, HEK-293T cells were cotransfected with packaging plasmid psPAX2 (Addgene #12260) and enveloping plasmid pMD2.G (Addgene #12259) using Calfectin (SignaGen, Rockville, MD). Lentiviral particles were collected, concentrated, and the functional titer was assessed using the EGFRt marker. PBMCs from healthy donors were stimulated for 24 hours using anti-CD3/anti-CD28 antibody-coated beads in the presence of interleukin 2 (Invitrogen, Carlsbad, CA). To track cell count and viability, viable cells were counted using trypan blue.

### Cell Killing Studies

Cytolytic assays were performed using luciferase-expressing cell lines and T cells transduced with GPC3 CARs as described previously (Wang et al., Blood 118(5):1255-1263, 2011). Briefly, tumor cells were incubated with T cells at indicated effector-target ratios for 24 hours. Luminescence of the lysates was analyzed using the luciferase assay system (Promega) on a plate spectrophotometer (Victor, Perkin Elmer). Specific lysis of each sample was calculated using the luminescence of target cells alone, corresponding to 0% lysis and 100% lysis respectively.

### Animal Studies

Xenograft mouse models were established by intraperitoneally injecting 3×10⁶ Hep3B or 1×10⁶ Huh7 GFP-luciferase cells in NOD-SCID-Gamma (NSG) mice. When the mean tumor bioluminescence reached 1×10⁸, the mice were injected intraperitoneally or intravenously with 5×10⁶ with untransduced normal human donor T cells or engineered HN3 CAR T cells. The treated mice were imaged biweekly during weeks 1-4 and weekly after week 4 on an a Xenogen IVIS-200 Spectrum camera. Mice were euthanized when they reached IACUC approved morbidity endpoints including any sign of distress.

### Example 2: HN3 Nanobody CAR Jurkat Cells Retain Antigen Specificity

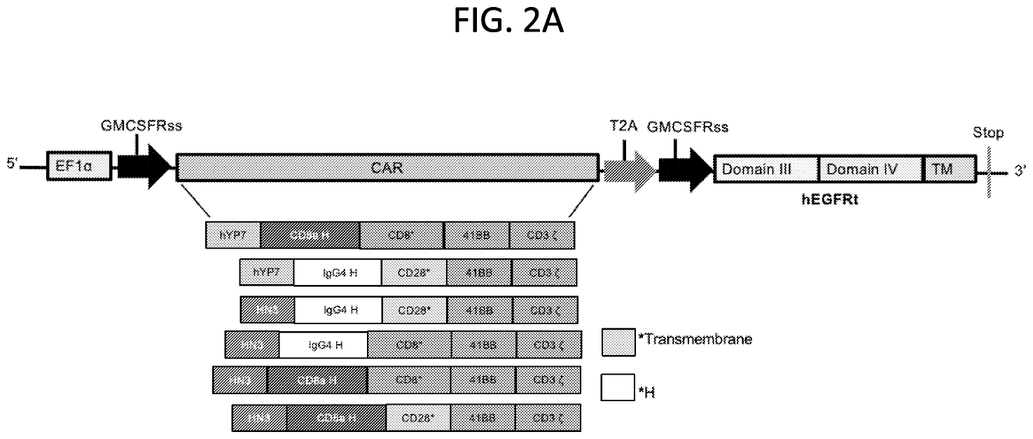

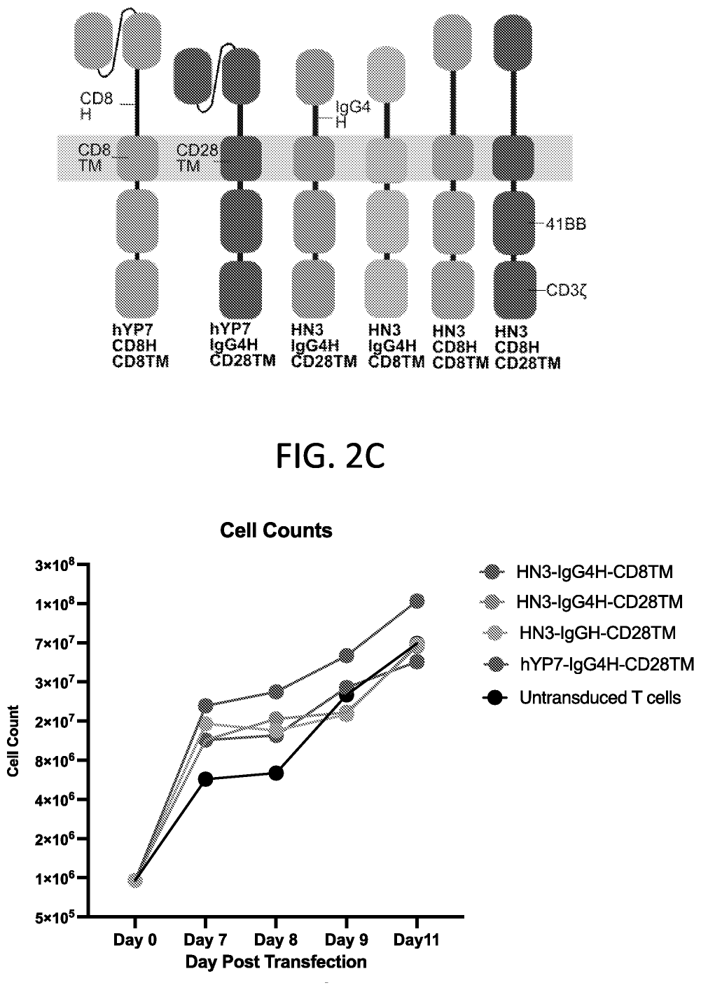

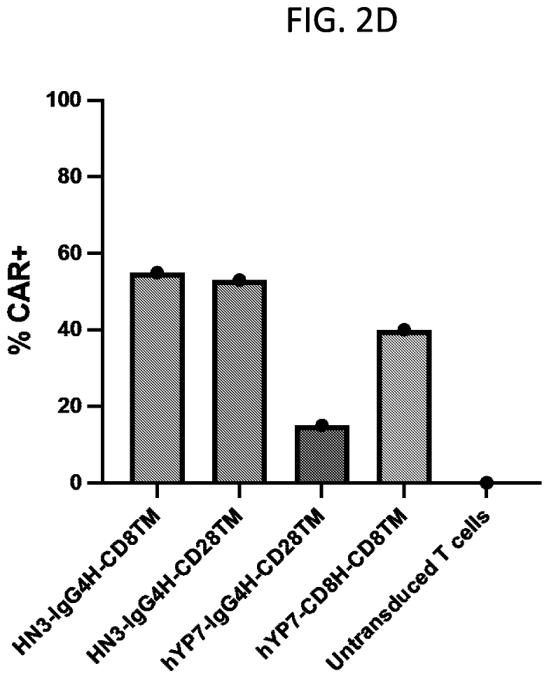

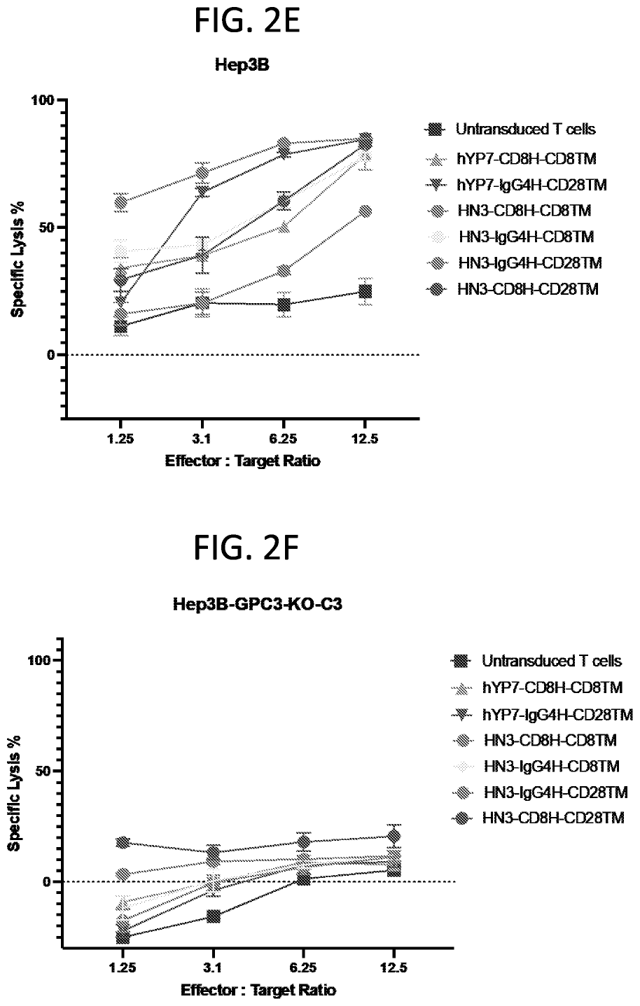

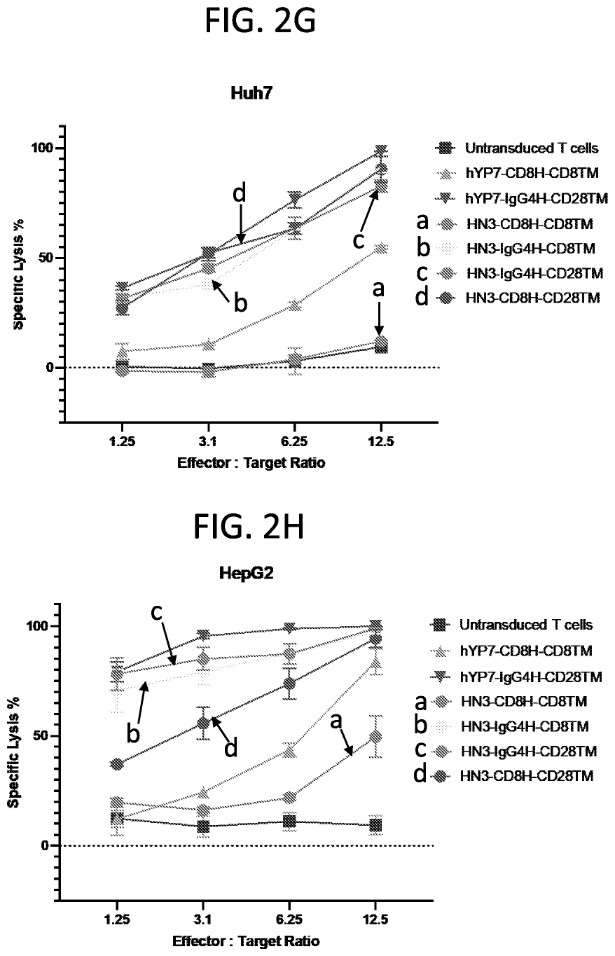

Several CAR constructs were generated using (1) HN3 or hYP7 scFv as the antigen-binding domain, (2) a modified hinge region derived from IgG4 (IgG4H) or a hinge region from CD8 (CD8H), and (3) a transmembrane domain from CD8 (CD8TM) or CD28 (CD28TM): HN3-CD8H-CD8TM, HN3-IgG4H-CD28TM, HN3-IgG4H-CD8TM, HN3-CD8H-CD28TM, hYP7-CD8H-CD8TM and hYP7-IgG4H-CD88TM. These CAR constructs are depicted in FIG. 2A and FIG. 2B. T cells were transduced with CAR-containing lentiviruses and cell counts were measured up to 11 days post-transfection (FIG. 2C). Cell counts of transduced T cells did not significantly differ from untransduced T cells. Transduction efficiency of the CAR constructs was also measured by detecting CAR-positive cells at Day 8 (FIG. 2D).

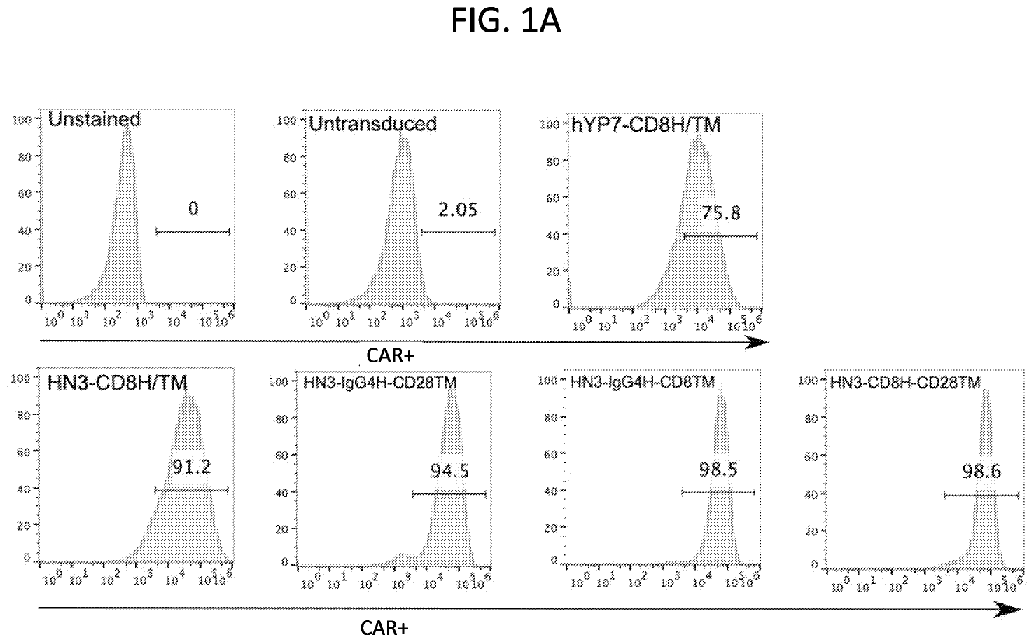

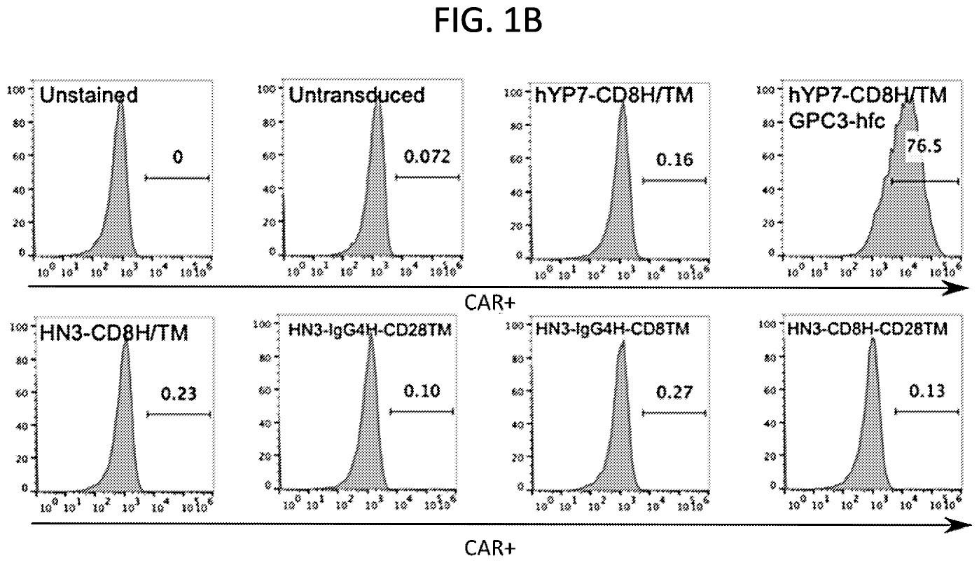

To test antigen specificity and cell binding capacity of the engineered GPC3-targeted CARs, T cell lines (Jurkat) transfected with the CAR constructs were developed and binding to GPC3 tagged with human Fc (GPC3-hFc) was assessed. First, successful transduction (95-98%) of all of the CAR constructs was confirmed using the truncated EGFR marker expressed by the CAR lentivirus. Next, it was demonstrated that all Jurkat-CAR constructs bind to GPC3-hFc efficiently (75.8-98.6%) at 1, 2 and 5 μg/mL (FIG. 1A). All CAR T cells specifically bound to GPC3-hFc (FIG. 1A) and not GPC1-hFc (FIG. 1B) at the same concentrations. Overall, this data indicates that HN3 and engineered HN3 CARs bind GPC3 specifically with minimal non-specific binding to other glypicans.

### Example 3: HN3-IgG4H-CD28TM CAR T Cells Enhance Activity in High and Low Antigen Density Settings In Vitro

To test CAR T cell potency in high versus low antigen density contexts, multiple HCC cell lines (Hep3B, HepG2 and Huh7) with various antigen densities were used (Fu et al, Hepatology 70(2):563-576, 2019). These cell lines were isolated from diverse patients with different levels of tumor aggression. Hep3B cells highly express GPC3, while Huh7 cells express GPC3 to a lesser extent. It was found that while HN3-IgG4H-CD28TM CAR T cells displayed the highest cell killing in all HCC lines, HN3-IgG4H-CD28TM CAR T cells showed high activity in low antigen density Huh7 cells (FIGS. 2E-2H). HN3-CD8H-CD28TM CAR T cells demonstrated 40% improved killing relative to HN3-CD8H-CD8TM CAR T cells, indicating that the CD28TM increases CAR T potency under both high and low antigen density conditions. However, the CD28TM alone is not sufficient to induce 100% killing at lower ratios. Comparing HN3-CD8H-CD28TM to HN3-IgG4H-CD28TM, the IgG4H increased potency by 10-20%. All CAR T cells minimally reacted with the GPC3 knockout cell line (FIG. 2F), indicating low levels of alloreactivity and antigen specific cell killing under these conditions. Taken together, these results indicate that HN3-IgG4H-CD28TM is the most potent CAR construct tested. Both the IgG4 hinge and CD28 TM independently enhance the anti-tumor activity of GPC3 CAR T cell therapy.

### Example 4: IgG4H and CD28TM Domains without Fc Enhance HN3 Potency In Vitro

To test whether the length of the hinge affects antigen binding, Fc components were incorporated and HN3 CAR T cells with short (IgG4H), medium (IgG4-CH3) and long hinges (IgG4-CH2CH3) were tested (Smith et al., Sci Transl Med. 11(485):eaau7746, 2019; Jonnalagadda et al., Mol Ther 23(4):757-768, 2015). Cell counts and transduction assays indicated that the long hinge construct was slower to expand and less effectively expressed at the same MOI. All constructs minimally reacted with the GPC3 knockout Hep3B ells, indicating an antigen-specific interaction. The HN3-IgG4H-CD28TM CAR T cells outperformed intermediate and long hinge CAR T cells. These data demonstrate that extension of the hinge length using Fc is not necessary and possibly impedes the HN3-GPC3 interaction in vivo. Altogether, this data confirmed that the HN3 nanobody can be engineered to induce potent killing in vitro using IgG4H and CD28TM together.

### Example 5: HN3-IgGH-CD28TM CAR T Cells Eradicate High GPC3 HCC Xenografts in Mice

Having demonstrated both the specificity and cytotoxicity of engineered CAR T cells in vitro, additional studies were performed to test in vivo antitumor activity. In the first study (FIG. 3A), Hep3B GFP/luciferase expressing cells (3 million) were injected IP into NSG mice and allowed to engraft for 12 days. Mice were treated with 5 million hYP7-CD8H-CD8TM, hYP7-IgG4H-CD28TM, HN3-IgG4H-CD8TM or HN3-IgG4H-CD28TM CAR T cells on Day 0 and imaged regularly. The results are shown in FIGS. 3B-3D. In line with the in vitro findings, HN3-IgG4H-CD28TM CAR T cells demonstrated high antitumor activity and the tumors were eradicated within 10 days. In contrast, tumors in the HN3-IgG4H-CD8TM group responded partially or continued to grow during the study. Median survival of mice treated with untransduced T cells, HN3-CD8H-CD8TM, and hYP7-CD8H-CD8TM CAR T cells was 27, 29, and 30 days, respectively. In contrast, three mice treated with HN3-IgG4H-CD28TM and HN3-IgG4H-CD8TM survived until the end of study (Day 35). The HN3-IgG4H-CD28TM group demonstrated swift reduction in tumor size starting at Day 3 and ultimately remained tumor free for full study period.

To address the issue of the scFv versus nanobody performance, hYP7-IgG4H-CD28TM CAR T cells were included to compare with HN3-IgG4H-CD28TM. The hYP7-IgGH-CD28TM construct was difficult to transduce in vitro with 25-30% transduction. Despite these challenges, the construct performed well in cell testing particularly in the Hep3B and HepG2 lines. Surprisingly, in vivo, the hYP7 construct induced delayed tumor regression at Day 30 while the engineered HN3 construct induced tumor regression within 3 days. These data suggest that HN3-IgG4-CD28TM is more potent than h YP7-IgG4-CD28TM at the same dose level.

In a second study using the same study conditions described above (FIG. 4A), the in vivo antitumor activity of Fc containing CAR T cells was tested. The results are shown in FIGS. 4B-4D. Consistent with the in vitro findings, in the HN3-IgG4H-CD28TM group, tumors regressed within 7 days. All tumors in the HN3-IgG4H-CD28TM group were eradicated entirely and did not regrow after 67 days. Mice treated with CAR T cells containing Fc components showed partial responses. In particular, 4 of 5 mice in the HN3-IgG4H-CH3-CD28TM group (“HN3-IgG4H-CD28TM-M”) had no tumors at Day 24 and 2 of 5 mice in the HN3-IgG4H-CH2CH3-CD28TM (“HN3-IgG4H-CD28TM-L”) group had no tumors at Day 15. The HN3-CD8H-CD8TM group displayed no response to CAR T cell treatment.

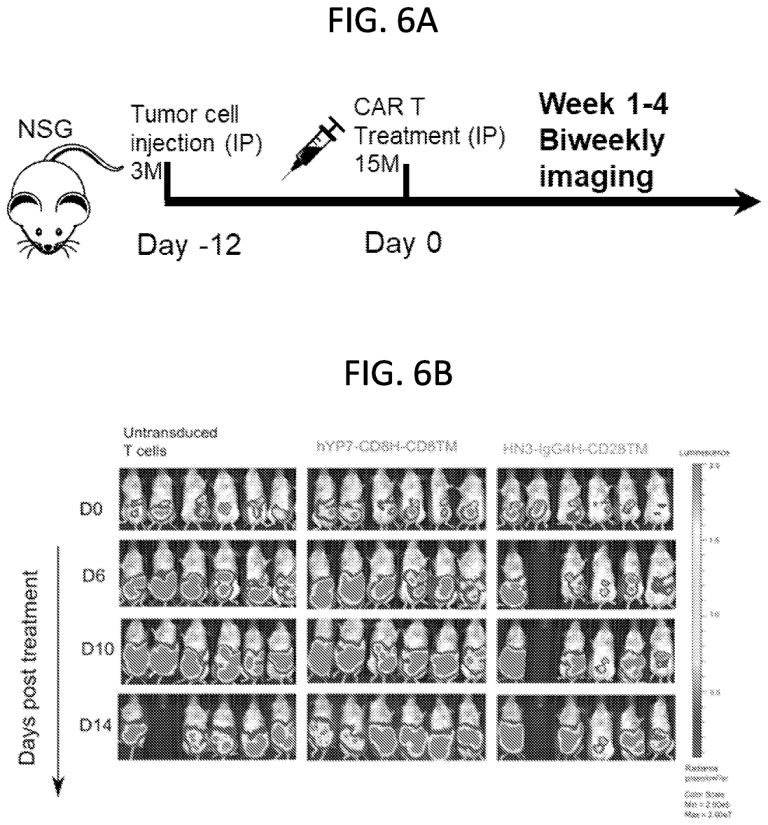

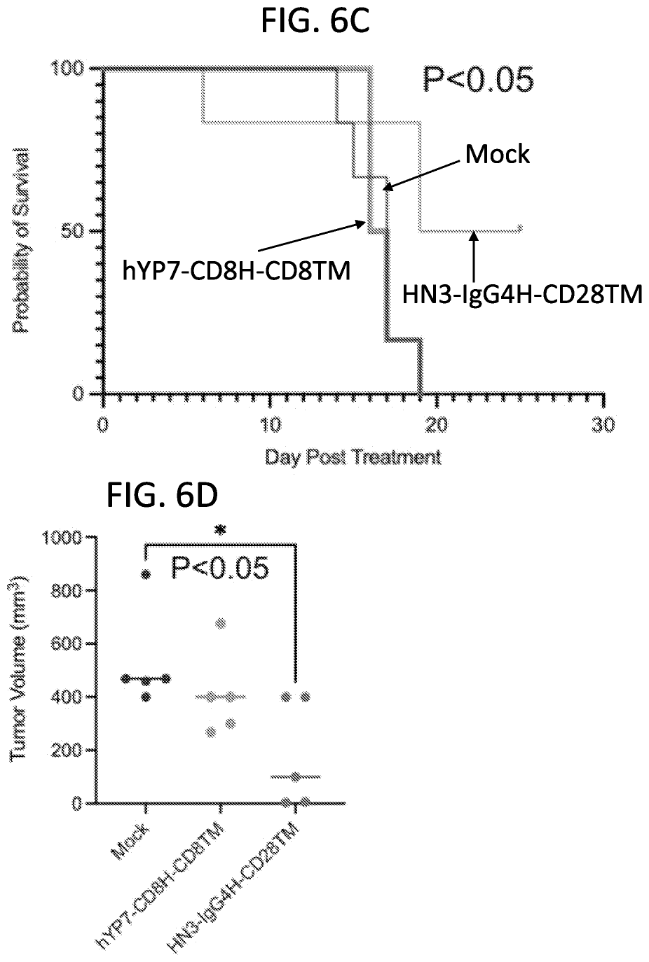

In a low antigen density model in vivo (Huh 7) a striking tumor regression was observed in five of six mice (FIGS. 6A-6D). All together, these data indicated that the HN3-IgG4H-CD28TM construct was the most potent in achieving a rapid and durable antitumor response.

### Example 6: Chimeric Antigen Receptor T Cells Targeting GPC3 Display Enrichment of CD8+ and Tₑₘᵣₐ Subsets

To monitor the number of CAR T cells, exhaustion markers and T cell subsets, blood from study mice was analyzed (Chen et al., Cancer Discov 11(9):2186-2199, 2021; Ma et al., Cancer Discov 3(4):418-429, 2013; Xu et al., Blood 123(24):3750-3759, 2014). The results are shown in FIGS. 5A-5F. At 28 days, there were several log fold more CAR T cells in mice from the HN3-IgG4H-CD28TM group than the HN3-CD8H-CD8TM treated group, indicating a large, proliferative response. Further, PDI expression was lowest in the HN3-IgG4H-CH3-CD28TM group (Guo et al., Front Pharmacol 9:1118, 2018). To understand the T cell response, the CD8 versus CD4 expression was examined, as well as T cell subsets including Tscm, Tem, Tem and Temra. The engineered CAR T cells harbored a vast number of CAR-positive CD8 T cells. The T cells, while initially showing similar phenotypes early on, displayed both memory and effector function in vivo by weeks 4 and 5. By weeks 4 and 5, most of the CAR T cells in the HN3-IgG4H-CD28TM group had the CD8+ phenotype and engaged in effector functions. These data indicate that expansion of the CAR T cells over a 2-to-5-week period produced a clear transition to CD8+ Tₑₘᵣₐ, which appear to be very important to the antitumor response. Central memory T cells also remained within a subset of the population indicating that both types of T cells are necessary to avert exhaustion and continue trafficking to the tumor site. This preponderance of CD8+Tₑₘᵣₐ was not seen in the non-responders in the Huh7 tumor group. Together, this supports the hypothesis that early transition of CD8+ Tₑₘᵣₐ cells are important for inducing regression and durable responses.

### Example 7: HN3-IgG4H-CD28TM Block Wnt Signaling and Activate NFAT in HCC

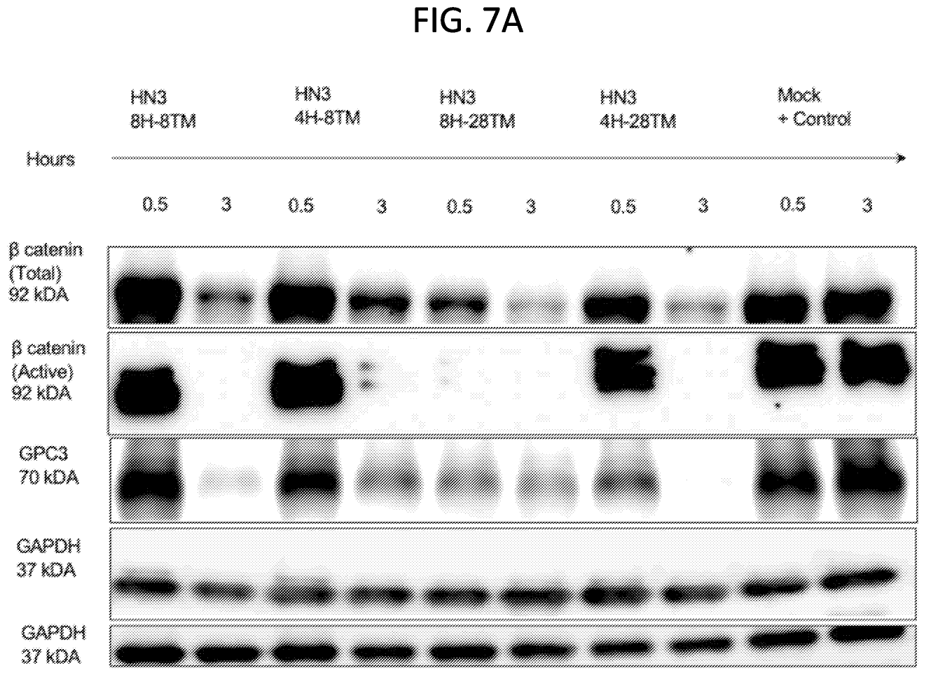

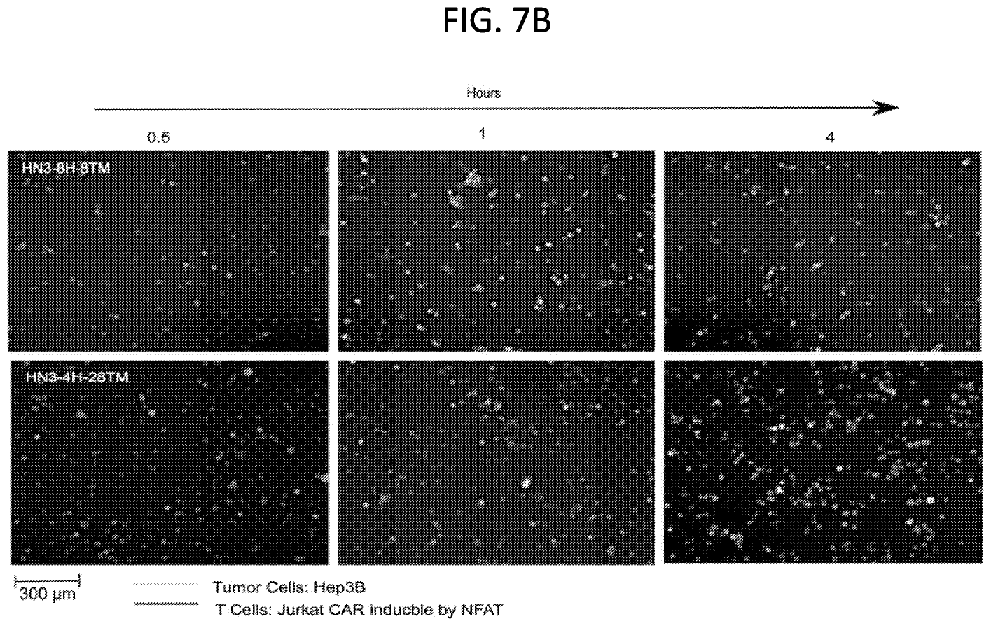

Due to the potency observed in vivo, the ability of the HN3 constructs to block tumor Wnt signaling when exposed to GPC3 expressing Hep3B cells was examined (FIG. 7A). A reduction in both active and total β catenin levels were observed for HN3-IgG4H-CD28TM CAR T cells when cocultured with Hep3B cells at 30 minutes and 3 hours. The engineered HN3 CAR T cells swiftly inhibited total β catenin and abolished active β catenin entirely at 3 hours (FIG. 7A). Even at 30 minutes, engineered HN3 constructs had lower levels of total β catenin. These data confirmed that Wnt signaling is a direct target of the HN3-based CAR T cells and that within 3 hours HN3 directed CAR T cells directly inhibit β catenin activation.

To understand how the CAR T cells act in vivo, a study was performed to determine if NFAT played a role in the T cell signaling (Wilson et al., Nat Commun 6:6818, 2015; Li et al., Cancer Cell 31:383-395, 2017; Capurro et al., J Cell Sci 127:1565-1575, 2014). Over each time point, gradually more NFAT signaling was observed in the HN3-IgG4H-CD28TM CAR T cells compared to the original construct containing CD8H-CD8TM. Saturation of NFAT signaling reached a maximum at 4 hours when there were more CAR cells in the representative imaging window than Hep3B cells (FIG. 7B). When the tdTomato signal was quantified by cell count and size, the differences between NFAT signaling in HN3-IgG4H-CD28TM versus HN3-CD8H-CD8TM were striking. NFAT signaling in HN3-IgG4H-CD28TM treated Hep3B cells begins at a higher cell number in the 200 range overall and doubles within 4 hours with respect to cell number and intensity of the signal. These observations indicated that signaling via NFAT is more prevalent in T cell activation in HN3-IgG4H-CD28TM than HN3-CD8H-CD8TM. Overall, it was observed that NFAT is steadily upregulated in the HN3-IgG4H-CD28TM CAR T cells. Based on the full body of data, it is proposed that fine tuning the antigen-CAR interaction leads to these mechanistic changes in the cell thereby leading to potent and durable eradication of tumor.

It will be apparent that the precise details of the methods or compositions described may be varied or modified without departing from the spirit of the described aspects of the disclosure. We claim all such modifications and variations that fall within the scope and spirit of the claims below.

1. A chimeric antigen receptor (CAR), comprising:
an extracellular antigen-binding domain that specifically binds glypican-3 (GPC3);
a hinge region consisting of the IgG4 hinge region set forth as SEQ ID NO: 43 or SEQ ID NO: 52;
a transmembrane domain;
an intracellular co-stimulatory domain; and
an intracellular signaling domain.

2. The CAR of claim 1, wherein the extracellular antigen-binding domain comprises a GPC3-specific single-domain antibody or a GPC3-specific single chain variable fragment (scFv).

3. The CAR of claim 2, wherein:
the single-domain antibody comprises the complementarity determining region 1 (CDR1), CDR2 and CDR3 sequences of SEQ ID NO: 18; or
the scFv comprises a variable heavy (VH) domain and a variable light (VL) domain and the VH domain comprises the complementarity determining region 1 (CDR1), CDR2 and CDR3 sequences of SEQ ID NO: 20, and the VL domain comprises the CDR1, CDR2 and CDR3 sequences of SEQ ID NO: 22.

4. The CAR of claim 3, wherein the CDR1, CDR2 and CDR3 sequences of the single-domain antibody respectively comprise:
residues 31-35, 50-65 and 96-105 of SEQ ID NO: 18; or
residues 26-33, 51-57 and 96-105 of SEQ ID NO: 18.

5. The CAR of claim 3, wherein:
the amino acid sequence of the single-domain antibody is at least 90% identical to SEQ ID NO: 18 and comprises the CDR1, CDR2 and CDR3 sequences of SEQ ID NO: 18; or
the amino acid sequence of the VH domain is at least 90% identical to SEQ ID NO: 20 and comprises the CDR1, CDR2 and CDR3 sequences of SEQ ID NO: 20, and the amino acid sequence of the VL domain is at least 90% identical to SEQ ID NO: 22 and comprises the CDR1, CDR2 and CDR3 sequences of SEQ ID NO: 22.

6. The CAR of claim 3, wherein:
the amino acid sequence of the single-domain antibody comprises or consists of SEQ ID NO: 18; or
the amino acid sequence of the VH domain comprises or consists of SEQ ID NO: 20 and the amino acid sequence of the VL domain comprises or consists of SEQ ID NO: 22.

7-8. (canceled)

9. The CAR of claim 3, wherein:
the VH domain CDR1, CDR2 and CDR3 sequences respectively comprise residues 31-35, 50-68 and 101-106 of SEQ ID NO: 20 and the VL domain CDR1, CDR2 and CDR3 sequences respectively comprise residues 24-40, 56-62 and 95-103 of SEQ ID NO: 22; or
the VH domain CDR1, CDR2 and CDR3 sequences respectively comprise residues 26-33, 51-60 and 99-106 of SEQ ID NO: 20 and the VL domain CDR1, CDR2 and CDR3 sequences respectively comprise residues 27-38, 56-58 and 95-103 of SEQ ID NO: 22.

10-11. (canceled)

12. The CAR of claim 9, wherein the amino acid sequence of the scFv comprises residues 1-245 of SEQ ID NO: 14.

13. The CAR of claim 1, wherein:
the transmembrane domain comprises a CD28 transmembrane domain;
the co-stimulatory domain comprises a 4-1BB signaling moiety; and/or
the signaling domain comprises a CD3 signaling domain.

14-15. (canceled)

16. An isolated cell expressing the CAR of claim 1.

17. The isolated cell of claim 16, which is an immune cell or an induced pluripotent stem cell (iPSC).

18. The isolated cell of claim 17, wherein the immune cell is a T cell, a B cell, a natural killer (NK) cell or a macrophage.

19. A nucleic acid molecule encoding the CAR of claim 1.

20. The nucleic acid molecule of claim 19, operably linked to a promoter.

21. The nucleic acid molecule of claim 19, comprising in the 5′ to 3′ direction:
a nucleic acid encoding a first granulocyte-macrophage colony stimulating factor receptor signal sequence (GMCSFRss);
a nucleic acid encoding the antigen-binding domain;
a nucleic acid encoding the IgG4 hinge region;
a nucleic acid encoding the transmembrane domain;
a nucleic acid encoding the co-stimulatory domain;
a nucleic acid encoding the signaling domain;
a nucleic acid encoding a self-cleaving 2A peptide;
a nucleic acid encoding a second GMCSFRss; and
a nucleic acid encoding a truncated human epidermal growth factor receptor (huEGFRt).

22. The nucleic acid molecule of claim 21, further comprising a human elongation factor 1α (EF1α) promoter sequence 5′ of the nucleic acid encoding the first GMCSFRss.

23. A vector comprising the nucleic acid molecule of claim 19.

24. The vector of claim 23, wherein the vector is a lentiviral vector.

25. An isolated cell comprising the nucleic acid molecule of claim 19.

26. The isolated cell of claim 25, which is an immune cell or an induced pluripotent stem cell (iPSC).

27. The isolated cell of claim 26, wherein the immune cell is a T cell, a B cell, an NK cell or a macrophage.

28. A composition comprising a pharmaceutically acceptable carrier and the cell of claim 16.

29. A method of treating a GPC3-positive cancer in a subject, or a method of inhibiting tumor growth or metastasis of a GPC3-positive cancer in a subject, comprising administering to the subject a therapeutically effective amount of the cell of claim 16.

30. (canceled)

31. The method of claim 29, wherein the GPC3-positive cancer is a solid tumor.

32. The method of claim 29, wherein the GPC3-positive cancer is a hepatocellular carcinoma (HCC), melanoma, ovarian clear-cell carcinoma, yolk sac tumor (YST), neuroblastoma, hepatoblastoma, Wilms' tumor, squamous cell carcinoma of the lung, testicular nonseminomatous germ cell tumor, liposarcoma, cervical intraepithelial neoplasia, adenoma of the adrenal gland, schwannoma or embryonal tumor.

33. (canceled)


SEQUENCE LISTING (60 sequences)

SEQ ID NO: 1 - DNA, 552 bp, synthetic construct
  misc_feature 1..552: note = Synthetic nucleic acid
  source 1..552: mol_type = other DNA
caggtgcagctggtgcagtctgggggaggcttggtacagcctggagggtccctgagactctcctgtgcagcctcttatttcgatttcgattcttatgaaatgagctgggtccgccaggctccagggaagggcctagagtggattgggagtatctatcatagtgggagcacctactacaacccgtccctcaagagtcgagtcaccatctccagagacaattccaagaacacgctgtatctgcaaatgaacaccctgagagccgaggacacagccacgtattactgtgcgagagtaaatatggaccgatttgactactggggccagggaaccctggtcaccgtctcctcaactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgacatctacatctgggcgcccttggccgggacttgtggggtccttctcctgtcactggttatcacc

SEQ ID NO: 2 - protein, 184 aa, synthetic construct
  REGION 1..184: note = Synthetic polypeptide
QVQLVQSGGGLVQPGGSLRLSCAASYFDFDSYEMSWVRQAPGKGLEWIGSIYHSGSTYYNPSLKSRVTISRDNSKNTLYLQMNTLRAEDTATYYCARVNMDRFDYWGQGTLVTVSSTSTTTPAPRPPTPAPTIASQPLSLRPEACRPAAGGAVHTRGLDFACDIYIWAPLAGTCGVLLLSLVIT

SEQ ID NO: 3 - DNA, 570 bp, synthetic construct
  misc_feature 1..570: note = Synthetic nucleic acid
  source 1..570: mol_type = other DNA
caggtgcagctggtgcagtctgggggaggcttggtacagcctggagggtccctgagactctcctgtgcagcctcttatttcgatttcgattcttatgaaatgagctgggtccgccaggctccagggaagggcctagagtggattgggagtatctatcatagtgggagcacctactacaacccgtccctcaagagtcgagtcaccatctccagagacaattccaagaacacgctgtatctgcaaatgaacaccctgagagccgaggacacagccacgtattactgtgcgagagtaaatatggaccgatttgactactggggccagggaaccctggtcaccgtctcctcaactagtaccactactcctgcgcctagaccacccacacctgcccctacgatcgcttcacaaccgcttagccttaggccggaagcatgtcggccagcggcggggggcgcagtgcacacgagggggctggacttcgcctgtgacttctgggtgctggtggtggtgggaggcgtgctggcctgctattccctgctggtgaccgtggccttcatcatcttttgggtg

SEQ ID NO: 4 - protein, 190 aa, synthetic construct
  REGION 1..190: note = Synthetic polypeptide
QVQLVQSGGGLVQPGGSLRLSCAASYFDFDSYEMSWVRQAPGKGLEWIGSIYHSGSTYYNPSLKSRVTISRDNSKNTLYLQMNTLRAEDTATYYCARVNMDRFDYWGQGTLVTVSSTSTTTPAPRPPTPAPTIASQPLSLRPEACRPAAGGAVHTRGLDFACDFWVLVVVGGVLACYSLLVTVAFIIFWV

SEQ ID NO: 5 - DNA, 471 bp, synthetic construct
  misc_feature 1..471: note = Synthetic nucleic acid
  source 1..471: mol_type = other DNA
caggtgcagctggtgcagtctgggggaggcttggtacagcctggagggtccctgagactctcctgtgcagcctcttatttcgatttcgattcttatgaaatgagctgggtccgccaggctccagggaagggcctagagtggattgggagtatctatcatagtgggagcacctactacaacccgtccctcaagagtcgagtcaccatctccagagacaattccaagaacacgctgtatctgcaaatgaacaccctgagagccgaggacacagccacgtattactgtgcgagagtaaatatggaccgatttgactactggggccagggaaccctggtcaccgtctcctcaactagtgagagcaagtacggaccaccttgcccaccatgtccattctgggtgctggtggtggtgggaggcgtgctggcctgctattccctgctggtgaccgtggccttcatcatcttttgggtg

SEQ ID NO: 6 - protein, 157 aa, synthetic construct
  REGION 1..157: note = Synthetic polypeptide
QVQLVQSGGGLVQPGGSLRLSCAASYFDFDSYEMSWVRQAPGKGLEWIGSIYHSGSTYYNPSLKSRVTISRDNSKNTLYLQMNTLRAEDTATYYCARVNMDRFDYWGQGTLVTVSSTSESKYGPPCPPCPFWVLVVVGGVLACYSLLVTVAFIIFWV

SEQ ID NO: 7 - DNA, 453 bp, synthetic construct
  misc_feature 1..453: note = Synthetic nucleic acid
  source 1..453: mol_type = other DNA
caggtgcagctggtgcagtctgggggaggcttggtacagcctggagggtccctgagactctcctgtgcagcctcttatttcgatttcgattcttatgaaatgagctgggtccgccaggctccagggaagggcctagagtggattgggagtatctatcatagtgggagcacctactacaacccgtccctcaagagtcgagtcaccatctccagagacaattccaagaacacgctgtatctgcaaatgaacaccctgagagccgaggacacagccacgtattactgtgcgagagtaaatatggaccgatttgactactggggccagggaaccctggtcaccgtctcctcaactagtgagagcaagtacggaccaccttgcccaccatgtccaatctacatctgggcgcccttggccgggacttgtggggtccttctcctgtcactggttatcacc

SEQ ID NO: 8 - protein, 151 aa, synthetic construct
  REGION 1..151: note = Synthetic polypeptide
QVQLVQSGGGLVQPGGSLRLSCAASYFDFDSYEMSWVRQAPGKGLEWIGSIYHSGSTYYNPSLKSRVTISRDNSKNTLYLQMNTLRAEDTATYYCARVNMDRFDYWGQGTLVTVSSTSESKYGPPCPPCPIYIWAPLAGTCGVLLLSLVIT

SEQ ID NO: 9 - DNA, 792 bp, synthetic construct
  misc_feature 1..792: note = Synthetic nucleic acid
  source 1..792: mol_type = other DNA
caggtgcagctggtgcagtctgggggaggcttggtacagcctggagggtccctgagactctcctgtgcagcctcttatttcgatttcgattcttatgaaatgagctgggtccgccaggctccagggaagggcctagagtggattgggagtatctatcatagtgggagcacctactacaacccgtccctcaagagtcgagtcaccatctccagagacaattccaagaacacgctgtatctgcaaatgaacaccctgagagccgaggacacagccacgtattactgtgcgagagtaaatatggaccgatttgactactggggccagggaaccctggtcaccgtctcctcaactagtgagtccaagtacggccccccttgcccaccatgtccaggacagccaagggagcctcaggtgtataccctgcctccatctcaggaggagatgaccaagaaccaggtgagcctgacatgcctggtgaagggcttctacccaagcgacatcgccgtggagtgggagtccaatggccagcccgagaacaattacaagaccacaccccctgtgctggactctgatggcagcttctttctgtattcccggctgaccgtggataagtctagatggcaggagggcaacgtgttcagctgctccgtgatgcacgaggccctgcacaatcactacacacagaagtctctgagcctgtccctgggcaagttttgggtgctggtggtggtgggaggcgtgctggcctgttattctctgctggtgacagtggccttcatcatcttttgggtg

SEQ ID NO: 10 - protein, 264 aa, synthetic construct
  REGION 1..264: note = Synthetic polypeptide
QVQLVQSGGGLVQPGGSLRLSCAASYFDFDSYEMSWVRQAPGKGLEWIGSIYHSGSTYYNPSLKSRVTISRDNSKNTLYLQMNTLRAEDTATYYCARVNMDRFDYWGQGTLVTVSSTSESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWV

SEQ ID NO: 11 - DNA, 1119 bp, synthetic construct
  misc_feature 1..1119: note = Synthetic nucleic acid
  source 1..1119: mol_type = other DNA
caggtgcagctggtgcagtctgggggaggcttggtacagcctggagggtccctgagactctcctgtgcagcctcttatttcgatttcgattcttatgaaatgagctgggtccgccaggctccagggaagggcctagagtggattgggagtatctatcatagtgggagcacctactacaacccgtccctcaagagtcgagtcaccatctccagagacaattccaagaacacgctgtatctgcaaatgaacaccctgagagccgaggacacagccacgtattactgtgcgagagtaaatatggaccgatttgactactggggccagggaaccctggtcaccgtctcctcaactagtgagtccaagtacggaccaccttgcccaccatgtccagcacctccagtggcaggaccaagcgtgttcctgtttccacctaagcctaaggacaccctgatgatctctcggaccccagaggtgacatgcgtggtggtggacgtgagccaggaggatccagaggtgcagttcaactggtacgtggatggcgtggaggtgcacaatgccaagacaaagcccagggaggagcagtttcagagcacctaccgcgtggtgtccgtgctgacagtgctgcaccaggactggctgaacggcaaggagtataagtgcaaggtgtccaataagggcctgcctagctccatcgagaagaccatctctaaggcaaagggacagccaagggagcctcaggtgtacacactgccacccagccaggaggagatgaccaagaaccaggtgtccctgacatgtctggtgaagggcttctatccctccgacatcgccgtggagtgggagtctaatggccagcctgagaacaattacaagaccacacctccagtgctggactccgatggctctttctttctgtattctcggctgaccgtggataagagcagatggcaggagggcaacgtgttctcttgcagcgtgatgcacgaggccctgcacaatcactacacacagaagtccctgtctctgagcctgggcaagttttgggtgctggtggtggtgggaggcgtgctggcctgttattccctgctggtgaccgtggccttcatcatcttttgggtg

SEQ ID NO: 12 - protein, 373 aa, synthetic construct
  REGION 1..373: note = Synthetic polypeptide
QVQLVQSGGGLVQPGGSLRLSCAASYFDFDSYEMSWVRQAPGKGLEWIGSIYHSGSTYYNPSLKSRVTISRDNSKNTLYLQMNTLRAEDTATYYCARVNMDRFDYWGQGTLVTVSSTSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWV

SEQ ID NO: 13 - DNA, 858 bp, synthetic construct
  misc_feature 1..858: note = Synthetic nucleic acid
  source 1..858: mol_type = other DNA
gaggtgcagcttgttgagtctggtggaggattggtgcagcctggagggtcattgagactctcatgtgcagcctctggattcaccttcaataagaatgccatgaattgggtccgccaggctccaggaaagggtttggaatgggttggccgcataagaaataaaactaataattatgcaacatattatgccgattcagtgaaagccaggtttaccatctccagagatgattcaaagaactcactctatctgcaaatgaacagcttgaaaaccgaggacacagccgtgtactattgtgtggctggtaactcgtttgcttactggggccaagggactctggtcactgtctctgcaggcggaggcggatcaggtggtggcggatctggaggtggcggaagcgacattgtgatgacccagtctccagactccctagctgtgtcactgggagagagggccactatcaactgcaagtccagtcagagccttttatatagcagcaatcaaaagaactacttggcctggtaccaacagaaaccagggcagcctcctaaactgctgatttactgggcatccagtagggaatctggggtccctgatcgcttcagtggcagtggatctgggacagatttcactctcaccatcagcagtctgcaggctgaagacgtggcagtttattactgtcagcaatattataactatccgctcacgttcggtcaggggaccaagttggagatcaaaactagtgagagcaagtacggaccaccttgcccaccatgtccattctgggtgctggtggtggtgggaggcgtgctggcctgctattccctgctggtgaccgtggccttcatcatcttttgggtg

SEQ ID NO: 14 - protein, 286 aa, synthetic construct
  REGION 1..286: note = Synthetic polypeptide
EVQLVESGGGLVQPGGSLRLSCAASGFTFNKNAMNWVRQAPGKGLEWVGRIRNKTNNYATYYADSVKARFTISRDDSKNSLYLQMNSLKTEDTAVYYCVAGNSFAYWGQGTLVTVSAGGGGSGGGGSGGGGSDIVMTQSPDSLAVSLGERATINCKSSQSLLYSSNQKNYLAWYQQKPGQPPKLLIYWASSRESGVPDRFSGSGSGTDFTLTISSLQAEDVAVYYCQQYYNYPLTFGQGTKLEIKTSESKYGPPCPPCPFWVLVVVGGVLACYSLLVTVAFIIFWV

SEQ ID NO: 15 - DNA, 939 bp, synthetic construct
  misc_feature 1..939: note = Synthetic nucleic acid
  source 1..939: mol_type = other DNA
gaggtgcagcttgttgagtctggtggaggattggtgcagcctggagggtcattgagactctcatgtgcagcctctggattcaccttcaataagaatgccatgaattgggtccgccaggctccaggaaagggtttggaatgggttggccgcataagaaataaaactaataattatgcaacatattatgccgattcagtgaaagccaggtttaccatctccagagatgattcaaagaactcactctatctgcaaatgaacagcttgaaaaccgaggacacagccgtgtactattgtgtggctggtaactcgtttgcttactggggccaagggactctggtcactgtctctgcaggcggaggcggatcaggtggtggcggatctggaggtggcggaagcgacattgtgatgacccagtctccagactccctagctgtgtcactgggagagagggccactatcaactgcaagtccagtcagagccttttatatagcagcaatcaaaagaactacttggcctggtaccaacagaaaccagggcagcctcctaaactgctgatttactgggcatccagtagggaatctggggtccctgatcgcttcagtggcagtggatctgggacagatttcactctcaccatcagcagtctgcaggctgaagacgtggcagtttattactgtcagcaatattataactatccgctcacgttcggtcaggggaccaagttggagatcaaaactagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgacatctacatctgggcgcccttggccgggacttgtggggtccttctcctgtcactggttatcacc

SEQ ID NO: 16 - protein, 313 aa, synthetic construct
  REGION 1..313: note = Synthetic polypeptide
EVQLVESGGGLVQPGGSLRLSCAASGFTFNKNAMNWVRQAPGKGLEWVGRIRNKTNNYATYYADSVKARFTISRDDSKNSLYLQMNSLKTEDTAVYYCVAGNSFAYWGQGTLVTVSAGGGGSGGGGSGGGGSDIVMTQSPDSLAVSLGERATINCKSSQSLLYSSNQKNYLAWYQQKPGQPPKLLIYWASSRESGVPDRFSGSGSGTDFTLTISSLQAEDVAVYYCQQYYNYPLTFGQGTKLEIKTSTTTPAPRPPTPAPTIASQPLSLRPEACRPAAGGAVHTRGLDFACDIYIWAPLAGTCGVLLLSLVIT

SEQ ID NO: 17 - DNA, 348 bp, synthetic construct
  misc_feature 1..348: note = Synthetic nucleic acid
  source 1..348: mol_type = other DNA
caggtgcagctggtgcagtctgggggaggcttggtacagcctggagggtccctgagactctcctgtgcagcctcttatttcgatttcgattcttatgaaatgagctgggtccgccaggctccagggaagggcctagagtggattgggagtatctatcatagtgggagcacctactacaacccgtccctcaagagtcgagtcaccatctccagagacaattccaagaacacgctgtatctgcaaatgaacaccctgagagccgaggacacagccacgtattactgtgcgagagtaaatatggaccgatttgactactggggccagggaaccctggtcaccgtctcctca

SEQ ID NO: 18 - protein, 116 aa, synthetic construct
  REGION 1..116: note = Synthetic polypeptide
QVQLVQSGGGLVQPGGSLRLSCAASYFDFDSYEMSWVRQAPGKGLEWIGSIYHSGSTYYNPSLKSRVTISRDNSKNTLYLQMNTLRAEDTATYYCARVNMDRFDYWGQGTLVTVSS

SEQ ID NO: 19 - DNA, 351 bp, synthetic construct
  misc_feature 1..351: note = Synthetic nucleic acid
  source 1..351: mol_type = other DNA
gaggtgcagcttgttgagtctggtggaggattggtgcagcctggagggtcattgagactctcatgtgcagcctctggattcaccttcaataagaatgccatgaattgggtccgccaggctccaggaaagggtttggaatgggttggccgcataagaaataaaactaataattatgcaacatattatgccgattcagtgaaagccaggtttaccatctccagagatgattcaaagaactcactctatctgcaaatgaacagcttgaaaaccgaggacacagccgtgtactattgtgtggctggtaactcgtttgcttactggggccaagggactctggtcactgtctctgca

SEQ ID NO: 20 - protein, 117 aa, synthetic construct
  REGION 1..117: note = Synthetic polypeptide
EVQLVESGGGLVQPGGSLRLSCAASGFTFNKNAMNWVRQAPGKGLEWVGRIRNKTNNYATYYADSVKARFTISRDDSKNSLYLQMNSLKTEDTAVYYCVAGNSFAYWGQGTLVTVSA

SEQ ID NO: 21 - DNA, 339 bp, synthetic construct
  misc_feature 1..339: note = Synthetic nucleic acid
  source 1..339: mol_type = other DNA
gacattgtgatgacccagtctccagactccctagctgtgtcactgggagagagggccactatcaactgcaagtccagtcagagccttttatatagcagcaatcaaaagaactacttggcctggtaccaacagaaaccagggcagcctcctaaactgctgatttactgggcatccagtagggaatctggggtccctgatcgcttcagtggcagtggatctgggacagatttcactctcaccatcagcagtctgcaggctgaagacgtggcagtttattactgtcagcaatattataactatccgctcacgttcggtcaggggaccaagttggagatcaaa

SEQ ID NO: 22 - protein, 113 aa, synthetic construct
  REGION 1..113: note = Synthetic polypeptide
DIVMTQSPDSLAVSLGERATINCKSSQSLLYSSNQKNYLAWYQQKPGQPPKLLIYWASSRESGVPDRFSGSGSGTDFTLTISSLQAEDVAVYYCQQYYNYPLTFGQGTKLEIK

SEQ ID NO: 23 - DNA, 351 bp, synthetic construct
  misc_feature 1..351: note = Synthetic nucleic acid
  source 1..351: mol_type = other DNA
gaggtgcagcttgttgagactggcggaggattggtgcagcctgaagggtcattgaaactctcatgtgcaacgtctggattcaacttcaataccaatgccatgaactgggtccgccaggctccaggaaagggtttggaatgggttgctcgcgtaagaaataaaactaataattatgcaacatattatgccgattcagtgaaagacaggttcaccatctccagagatgattcacaaagaatggtctttctgcaaatgaataacttgaaaactgaggacacagccatctattactgtgtggcggggaactcgtttgcttattggggccaagggactctggtcactgtctctcct

SEQ ID NO: 24 - protein, 117 aa, synthetic construct
  REGION 1..117: note = Synthetic polypeptide
EVQLVETGGGLVQPEGSLKLSCATSGFNFNTNAMNWVRQAPGKGLEWVARVRNKTNNYATYYADSVKDRFTISRDDSQRMVFLQMNNLKTEDTAIYYCVAGNSFAYWGQGTLVTVSP

SEQ ID NO: 25 - DNA, 339 bp, synthetic construct
  misc_feature 1..339: note = Synthetic nucleic acid
  source 1..339: mol_type = other DNA
gacattgtgatgtcacagtctccatcctccctagctgtgtcagttggagagagggttactatgaactgcaagtccagtcagagtcttttatatagtagcaatcaaaagaactacttggcctggtaccagcagaaaccagggcagtctcctaaactgctgatttactgggcatccactagggaatctggggtccctgatcgcttcacaggcagtgtatctgggacagatttcactctcaccatcagcagtgtgaaggctgaagacctggcagtttattactgtcagcaatattataactatccgctcacgttcggtgctgggaccaagctggagctgaaa

SEQ ID NO: 26 - protein, 113 aa, synthetic construct
  REGION 1..113: note = Synthetic polypeptide
DIVMSQSPSSLAVSVGERVTMNCKSSQSLLYSSNQKNYLAWYQQKPGQSPKLLIYWASTRESGVPDRFTGSVSGTDFTLTISSVKAEDLAVYYCQQYYNYPLTFGAGTKLELK

SEQ ID NO: 27 - DNA, 351 bp, synthetic construct
  misc_feature 1..351: note = Synthetic nucleic acid
  source 1..351: mol_type = other DNA
gaggtgcagcttgttggaagtggtggaggattggtgcagcctgaagggtcattgaaactctcatgtgcagcctctggattcaccttcaagaccaatgccatgaactgggtccgccaggctccaggaaagggtttggaatgggttgctcgcataagaaataaaactaataattatgcaacatattatgccgactcagtgaaagacaggttcaccatctccagagatgattcacaaagcatgctctatctgcaaatgaacaacttgaaaactgaagacacagccatgtatttctgtgtggccggtaactcgtttgcttactggggccaggggactctggtcactgtctctgca

SEQ ID NO: 28 - protein, 117 aa, synthetic construct
  REGION 1..117: note = Synthetic polypeptide
EVQLVGSGGGLVQPEGSLKLSCAASGFTFKTNAMNWVRQAPGKGLEWVARIRNKTNNYATYYADSVKDRFTISRDDSQSMLYLQMNNLKTEDTAMYFCVAGNSFAYWGQGTLVTVSA

SEQ ID NO: 29 - DNA, 339 bp, synthetic construct
  misc_feature 1..339: note = Synthetic nucleic acid
  source 1..339: mol_type = other DNA
gacattgtgatgtcacagtctccatcctccctagctgtgtcagttggagagaaggttactgtgaactgcaagtccagtcagagccttttatatagtaacaatcaaaagaactacttggcctggtaccagcagaaaccagggcagtctcctaaactgctgatttactgggcatcaactagggaatatggggtccctgatcgcttcacaggcagtggatctgggacagatttcactctcaccatcagcagtgtgaaggctgaagacctggcagtttattactgtcagcaatattataactatccgctcacgttcggtgctgggaccaagctggagctgaaa

SEQ ID NO: 30 - protein, 113 aa, synthetic construct
  REGION 1..113: note = Synthetic polypeptide
DIVMSQSPSSLAVSVGEKVTVNCKSSQSLLYSNNQKNYLAWYQQKPGQSPKLLIYWASTREYGVPDRFTGSGSGTDFTLTISSVKAEDLAVYYCQQYYNYPLTFGAGTKLELK

SEQ ID NO: 31 - DNA, 351 bp, synthetic construct
  misc_feature 1..351: note = Synthetic nucleic acid
  source 1..351: mol_type = other DNA
gaggtccagcttgttgagactggtggaggattggtgcagcctaaagggtcattgaaactctcatgtgcagcctctggattcaccttcaataccaatgccatgaactgggtccgccaggctccaggaaagggtttgcaatgggttgctcgcgtaagaaataaaactaataattatgcaacatattatgccgattccgtgaaagacaggttcaccatctccagagatgattcacaaagcatgctctatctgcaaatgaacaacttgaaaactgaagacacggccatttattactgtgtggggggtaactcgtttgcttactggggccaagggactctggtcactgtctctgca

SEQ ID NO: 32 - protein, 117 aa, synthetic construct
  REGION 1..117: note = Synthetic polypeptide
EVQLVETGGGLVQPKGSLKLSCAASGFTFNTNAMNWVRQAPGKGLQWVARVRNKTNNYATYYADSVKDRFTISRDDSQSMLYLQMNNLKTEDTAIYYCVGGNSFAYWGQGTLVTVSA

SEQ ID NO: 33 - DNA, 339 bp, synthetic construct
  misc_feature 1..339: note = Synthetic nucleic acid
  source 1..339: mol_type = other DNA
gacattgtgatgtcccagtctccatcctccctagctgtgtcagttggagagaaggttactatgagctgcaagtccagtcagagccttttatatagtaacaatcaaaagaactacttggcctggtaccaccagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatagctatccactcacgttcggtgctgggaccaagctggagctgaaa

SEQ ID NO: 34 - protein, 113 aa, synthetic construct
  REGION 1..113: note = Synthetic polypeptide
DIVMSQSPSSLAVSVGEKVTMSCKSSQSLLYSNNQKNYLAWYHQKPGQSPKLLIYWASTRESGVPDRFTGSGSGTDFTLTISSVKAEDLAVYYCQQYYSYPLTFGAGTKLELK

SEQ ID NO: 35 - DNA, 339 bp, synthetic construct
  misc_feature 1..339: note = Synthetic nucleic acid
  source 1..339: mol_type = other DNA
gacattgtgatgtcccagtctccatcctccctagctgtgtcagttggagagaaggttactatgagctgcaagtccagtcagagccttttatatagtaacaatcaaaagaactacttggcctggtaccaccagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatatctatccactcacgttcggtgctgggaccaagctggagctgaaa

SEQ ID NO: 36 - protein, 113 aa, synthetic construct
  REGION 1..113: note = Synthetic polypeptide
DIVMSQSPSSLAVSVGEKVTMSCKSSQSLLYSNNQKNYLAWYHQKPGQSPKLLIYWASTRESGVPDRFTGSGSGTDFTLTISSVKAEDLAVYYCQQYYIYPLTFGAGTKLELK

SEQ ID NO: 37 - DNA, 339 bp, synthetic construct
  misc_feature 1..339: note = Synthetic nucleic acid
  source 1..339: mol_type = other DNA
gacattgtgatgtcccagtctccatcctccctagctgtgtcagttggagagaaggttactatgagctgcaagtccagtcagagccttttatatagtaacaatcaaaagaactacttggcctggtaccaccagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatagctatccactcacgttcggtgctgggaccaagctggaaataaaa

SEQ ID NO: 38 - protein, 113 aa, synthetic construct
  REGION 1..113: note = Synthetic polypeptide
DIVMSQSPSSLAVSVGEKVTMSCKSSQSLLYSNNQKNYLAWYHQKPGQSPKLLIYWASTRESGVPDRFTGSGSGTDFTLTISSVKAEDLAVYYCQQYYSYPLTFGAGTKLEIK

SEQ ID NO: 39 - DNA, 348 bp, synthetic construct
  misc_feature 1..348: note = Synthetic nucleic acid
  source 1..348: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcaactattcagaagcagggtctgcctacacagtacgcagactccgtgaaggggcggttcaccatctccagagacaattccaagaacacgctgtatctgcaaatgaacagcctgagagccgaggacacggccgtatattactgtgcgaaaaatcgggctaagtttgactactggggccagggaaccctggtcaccgtctcgagc

SEQ ID NO: 40 - protein, 116 aa, synthetic construct
  REGION 1..116: note = Synthetic polypeptide
EVQLLESGGGLVQPGGSLRLSCAASGFTFSSYAMSWVRQAPGKGLEWVSTIQKQGLPTQYADSVKGRFTISRDNSKNTLYLQMNSLRAEDTAVYYCAKNRAKFDYWGQGTLVTVSS

SEQ ID NO: 41 - DNA, 321 bp, synthetic construct
  misc_feature 1..321: note = Synthetic nucleic acid
  source 1..321: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctataatgcatccatgttgcaaagtggggtcccatcaaggttcagtggcagtggatctgggacagatttcactctcaccatcagcagtctgcaacctgaagattttgcaacttactactgtcaacagaatcggggttttcctctgacgttcggccaagggaccaaggtggaaatcaaa

SEQ ID NO: 42 - protein, 107 aa, synthetic construct
  REGION 1..107: note = Synthetic polypeptide
DIQMTQSPSSLSASVGDRVTITCRASQSISSYLNWYQQKPGKAPKLLIYNASMLQSGVPSRFSGSGSGTDFTLTISSLQPEDFATYYCQQNRGFPLTFGQGTKVEIK

SEQ ID NO: 43 - protein, 12 aa, synthetic construct
  REGION 1..12: note = Synthetic polypeptide
ESKYGPPCPPCP

SEQ ID NO: 44 - protein, 22 aa, synthetic construct
  REGION 1..22: note = Synthetic polypeptide
MLLLVTSLLLCELPHPAFLLIP

SEQ ID NO: 45 - protein, 45 aa, synthetic construct
  REGION 1..45: note = Synthetic polypeptide
TTTPAPRPPTPAPTIASQPLSLRPEACRPAAGGAVHTRGLDFACD

SEQ ID NO: 46 - protein, 27 aa, synthetic construct
  REGION 1..27: note = Synthetic polypeptide
FWVLVVVGGVLACYSLLVTVAFIIFWV

SEQ ID NO: 47 - protein, 21 aa, synthetic construct
  REGION 1..21: note = Synthetic polypeptide
IYIWAPLAGTCGVLLLSLVIT

SEQ ID NO: 48 - protein, 42 aa, synthetic construct
  REGION 1..42: note = Synthetic polypeptide
KRGRKKLLYIFKQPFMRPVQTTQEEDGCSCRFPEEEEGGCEL

SEQ ID NO: 49 - protein, 112 aa, synthetic construct
  REGION 1..112: note = Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 18 aa, synthetic construct
  REGION 1..18: note = Synthetic polypeptide
EGRGSLLTCGDVEENPGP

SEQ ID NO: 51 - protein, 335 aa, synthetic construct
  REGION 1..335: note = Synthetic polypeptide
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 52 - protein, 12 aa, synthetic construct
  REGION 1..12: note = Synthetic peptide
ESKYGPPCPSCP

SEQ ID NO: 53 - DNA, 351 bp, synthetic construct
  misc_feature 1..351: note = Synthetic nucleic acid
  source 1..351: mol_type = other DNA
gaggtgcagcttgttgagactggtggaggaatggtgcagcctgaagggtcattgaaactctcatgtgcagcctctggattcaccttcaataagaatgccatgaattgggtccgccaggctccaggaaagggtttggaatgggttgctcgcataagaaataaaactaataattatgcaacatattatgccgattcagtgaaagccaggtttaccatctccagagatgattcacaaagcatgctctatctgcaaatgaacaacttgaaaattgaggacacagccatgtactattgtgtggctggtaactcgtttgcttactggggccaagggactctggtcactgtctctgca

SEQ ID NO: 54 - protein, 117 aa, synthetic construct
  REGION 1..117: note = Synthetic polypeptide
EVQLVETGGGMVQPEGSLKLSCAASGFTFNKNAMNWVRQAPGKGLEWVARIRNKTNNYATYYADSVKARFTISRDDSQSMLYLQMNNLKIEDTAMYYCVAGNSFAYWGQGTLVTVSA

SEQ ID NO: 55 - DNA, 339 bp, synthetic construct
  misc_feature 1..339: note = Synthetic nucleic acid
  source 1..339: mol_type = other DNA
gacattgtgatgtcacagtctccatcctccctagttgtgtcaattggagagaaggttactatgacctgcaagtccagtcagagccttttatatagcagcaatcaaaagaactacttggcctggtaccaacagaaaccagggcagtctcctaaactgctgatttactgggcatccagtagggaatctggggtccctgatcgcttcacaggcagtggatctgggacagatttcactctcaccatcagcagtgtgaaggctgaagacctggcagtttattactgtcagcaatattataactatccgctcacgttcggtgctgggaccaagttggagctgaaa

SEQ ID NO: 56 - protein, 113 aa, synthetic construct
  REGION 1..113: note = Synthetic polypeptide
DIVMSQSPSSLVVSIGEKVTMTCKSSQSLLYSSNQKNYLAWYQQKPGQSPKLLIYWASSRESGVPDRFTGSGSGTDFTLTISSVKAEDLAVYYCQQYYNYPLTFGAGTKLELK

SEQ ID NO: 57 - DNA, 351 bp, synthetic construct
  misc_feature 1..351: note = Synthetic nucleic acid
  source 1..351: mol_type = other DNA
gaggtgcagcttgttggaagtggtggaggattggtgcagcctgaagggtcattgaaactctcatgtgcagcctctggattcaccttcaagaccaatgccatgaactgggtccgccaggctccaggaaagggtttggaatgggttgctcgcataagaaataaaactaataattatgcaacatattatgccgactcagtgaaagacaggttcaccatctccagagatgattcacaaagcatgctctatctgcaaatgaacaacttgaaaactgaagacacagccatgtatttctgtgtggccggtaactcgtttgcttacttgggccaggggactctggtcactgtctctgca

SEQ ID NO: 58 - protein, 117 aa, synthetic construct
  REGION 1..117: note = Synthetic polypeptide
EVQLVGSGGGLVQPEGSLKLSCAASGFTFKTNAMNWVRQAPGKGLEWVARIRNKTNNYATYYADSVKDRFTISRDDSQSMLYLQMNNLKTEDTAMYFCVAGNSFAYLGQGTLVTVSA

SEQ ID NO: 59 - protein, 109 aa, synthetic construct
  REGION 1..109: note = Synthetic polypeptide
APPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAK

SEQ ID NO: 60 - protein, 110 aa, synthetic construct
  REGION 1..110: note = Synthetic polypeptide
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK